(19) United States
(12) Patent Application Publication
(10) Pub. No.: US 2026/0297563 A1
(43) Pub. Date: Oct. 1, 2026

(54) PROBE LIBRARIES AND KITS

(71) Applicant: Nautilus Subsidiary, Inc., Seattle, WA (US)

(72) Inventors: Sheri Kay WILCOX, Longmont, CO (US); Sonal TONAPI, Sunnyvale, CA (US); Vivekananda BUDAMAGUNTA, San Carlos, CA (US); Aimee SANFORD, San Mateo, CA (US); Michael Augusto DARCY, Fremont, CA (US); Robert GROTHE, San Jose, CA (US); Scott TABAKMAN, San Diego, CA (US); Rajesh KOTA, San Diego, CA (US); Phillip BRERETON, Belmont, CA (US); Eric SPENCE, San Jose, CA (US)

(21) Appl. No.: 19/629,320

(22) Filed: Mar. 26, 2026

Related U.S. Application Data
(60) Provisional application No. 63/779,984, filed on Mar. 28, 2025.

(51) Int. Cl. C12N 15/10; G01N 21/64

(52) U.S. Cl. CPC C12N 15/1065; G01N 21/6428; G01N 2021/6439

(57) ABSTRACT

Probe libraries for use in systems and methods related to analyte characterization are provided. The probe libraries may comprise a plurality of unique probe compositions that differ with respect to probe architecture, probe composition, and/or probe concentration. Provided probe libraries may be useful for characterization of analytes at single-analyte resolution.

### CROSS-REFERENCE

This application claims priority to U.S. Provisional Application No. 63/779,984, filed on Mar. 28, 2025, which is incorporated herein by reference in its entirety.

### BACKGROUND

Detectable probes are utilized in many assays for the characterization of analytes. Characterization of analytes can include identification and/or quantitation of unknown analytes, as well as the determination of physical properties of analytes (e.g., size, length, residue sequence, degree of branching, isoelectric point, pKa, etc.). Some assays may utilize a plurality of unique detectable probes in a serial fashion, such as sequential use of the plurality of unique detectable probes, multiplexing of two or more unique detectable probes, or combinations thereof.

A detectable probe can comprise a binding reagent that has been modified to provide a detectable signal that facilitates detection of the binding interactions of the detectable probe. A detectable probe can be modified with an optically-detectable label, such as a fluorophore or luminophore, that provides a spatially-resolvable signal, for example when the detectable probe is bound to a fixed address. A detectable probe can be further modified with other components that modify the nature of the probe's interactions.

### SUMMARY

In an aspect, provided herein is a kit comprising a first probe composition and a second probe composition, the first probe composition differing from the second probe composition with respect to probe architecture or fluid composition. In another aspect, provided herein is a kit comprising a first probe composition and a second probe composition, the first probe composition differing from the second probe composition with respect to probe concentration.

In another aspect, provided herein is a system, comprising: (a) a vessel, wherein the vessel comprises a plurality of analytes, (b) a probe library comprising a first probe composition and a second probe composition, and (c) a fluidic system, wherein the fluidic system is configured to deliver the first probe composition and the second probe composition to the vessel of the fluidic device, and wherein the fluidic system is further configured to perform the steps of:

(i) incubating the first probe composition with the plurality of analytes in the presence of a first binding condition, (ii) removing the first probe composition from the vessel of the fluidic cartridge, and (iii) after removing the first probe composition from the vessel, incubating the second probe composition with the plurality of analytes in the presence of a second binding condition, wherein the first binding condition differs from the second binding condition.

In another aspect, provided herein is a method, comprising: a) binding probes of a first probe composition to a first set of analytes of a plurality of analytes in the presence of a first binding condition, b) detecting the probes of the first probe composition bound to the first set of analytes, c) after binding the probes of the first probe composition to the first set of analytes, binding probes of a second probe composition to a second set of analytes of the plurality of analytes in the presence of a second binding condition, and d) detecting the probes of the second probe composition bound to the second set of analytes.

In another aspect, provided herein is a kit, comprising: a plurality of joined vessels, wherein each vessel is fluidically isolated from each other vessel of the plurality of vessels, and wherein the plurality of joined vessels comprises a plurality of probe compositions, wherein each vessel of the plurality of joined vessels comprises a unique probe composition of the plurality of probe compositions, and wherein: (i) each vessel of a first set of vessels of the plurality of joined vessels comprises a multi-affinity probe composition; and (ii) each vessel of a second set of vessels of the plurality of joined vessels comprises a single-affinity probe composition.

In another aspect, provided herein is a method of characterizing an analyte, comprising: (a) sequentially contacting each probe composition of a probe library, as set forth herein, with an array of single analytes, (b) for each probe composition contacted to the array of analytes, detecting presence or absence of binding of a probe of the probe composition to each of the single analytes of the array of analytes at single-analyte resolution, and (c) for each single analyte of the array of analytes, characterizing the single analyte based upon the presence or absence of binding of each probe composition to the single analyte.

In another aspect, provided herein is a system for analyte characterization, comprising: (a) an array of analytes, wherein each analyte of the array of analytes is individually addressable at single-analyte resolution, (b) a kit comprising a probe library, as set forth herein, (c) a fluidic system that provides fluidic communication between the array of analytes and each probe composition of the kit, (d) a detection device that detects presence or absence of a probe of the probe library co-localized with each single analyte of the array of analytes, and (e) a processor that receives detection data from the detection device and, based upon the detection data, characterizes each individual analyte of the array of analytes.

### INCORPORATION BY REFERENCE

All publications, items of information available on the internet, patents, and patent applications cited in this specification are herein incorporated by reference to the same extent as if each individual publication, patent, or patent application was specifically and individually indicated to be incorporated by reference. To the extent publications, items of information available on the internet, patents, or patent applications incorporated by reference contradict the disclosure contained in the specification, the specification is intended to supersede and/or take precedence over any such contradictory material.

### BRIEF DESCRIPTION OF DRAWINGS

FIG. 1 depicts a schematic of a probe kit comprising a library of probe compositions, in accordance with some embodiments.

FIGS. 2A, 2B, 2C, and 2D illustrate differing formats for providing a probe library in separate vessels, in accordance with some embodiments.

FIG. 3 shows a system that is configured to implement certain methods set forth herein, in accordance with some embodiments.

FIGS. 4A and 4B display probes with differing probe architectures, in accordance with some embodiments.

FIGS. 5A and 5B depict differing systems for transferring probe compositions of a probe library into a fluidics system, in accordance with some embodiments.

FIGS. 6A, 6B, 6C, and 6D illustrate configurations of probe libraries that interface with retention structures of systems set forth herein, in accordance with some embodiments.

FIGS. 7A, 7B, and 7C show differing patterns of utilization of probe compositions of a probe library, in accordance with some embodiments.

### DETAILED DESCRIPTION

The present disclosure provides probe libraries that may be useful in various analytical methods and systems, including analytical methods and systems that provide characterization of analytes at single-molecule resolution. A probe library may comprise a plurality of probe compositions. Each individual probe composition of the plurality of probe compositions may differ from each other probe composition of the plurality of probe compositions in at least one aspect. A probe library may be provided as a kit containing a plurality of spatially-separated vessels, in which each vessel contains only one probe composition of the plurality of probe compositions of the probe library. For example, a probe library can be provided in a plate format (e.g., a 96-well plate, 384-well plate, or 1536-well plate), in which each individual well of the plate contains a single probe composition. In another example, a probe library can be provided as a roll, a tape, or a pack, in which the roll, tape, or pack comprises individual compartments (e.g., blister packs), each individual compartment comprising a single probe composition.

The probe libraries of the present disclosure may be useful for analyte characterization assays that utilize multiple differing probes. Numerous analytical methods utilize differing probes with highly specific binding profiles. For example, certain methods of peptide sequencing (e.g., Edman degradation-type sequencing methods) utilize probes that are highly specific to the modified forms of certain terminal amino acids. Accordingly, such methods may utilize probe libraries containing at least 20 unique probes, each probe being specific to the modified form of 1 of the 20 naturally occurring amino acids. Certain other analytical methods utilize promiscuous affinity reagents. For example, a method for identifying full-length proteins at single-molecule resolution utilizing the binding profiles of proteins for promiscuous probes is provided in U.S. Pat. Nos. 10,473,654 and 11,282,586, each of which is incorporated by reference in their entireties. Such methods may utilize at least 100 unique probes to form binding profiles for individual proteins.

The probe compositions of a probe library may be formulated to facilitate probe detection for a particular detection modality. Each probe composition of a probe library may be formulated for a common detection modality. For example, if an analyte characterization assay utilizes total internal reflectance fluorescence microscopy (TIRFM) for detection of probe interactions, each probe composition of a probe library may be formulated to facilitate TIRFM detection. The formulations of this TIRFM probe library may differ from the formulations of a probe library designed to perform an identical set of analyte characterizations via confocal fluorescence microscopy. The appropriateness of a probe composition for a particular detection modality can depend upon the chemical composition of the binding targets, the chemical composition of the probes of the probe composition, and the binding thermodynamics and/or binding kinetics of the probes with the binding targets.

In contrast to commercially-available reagents such as commercial antibodies that are sold in a standard storage buffer, a probe library for an analyte characterization assays may contain a plurality of individual probe compositions that differ from each other in some respect. Techniques for optimizing a particular probe composition for a particular assay are known in the art. Accordingly, the skilled person will recognize that each probe composition provided to a probe library can be individually optimized for an analyte characterization assay. In some cases, each probe composition of a plurality of probe compositions can be individually optimized (i.e., 20 probe compositions that differ with respect to fluid formulation, thereby giving 20 different fluid formulations). In other cases, a probe library can be provided to a set of differing formulations (e.g., 5 different probe architectures, 5 different fluid compositions, 5 different probe concentrations), and each unique affinity reagent of a probe library can be formulated according to the most optimal formulation amongst the set of differing formulations.

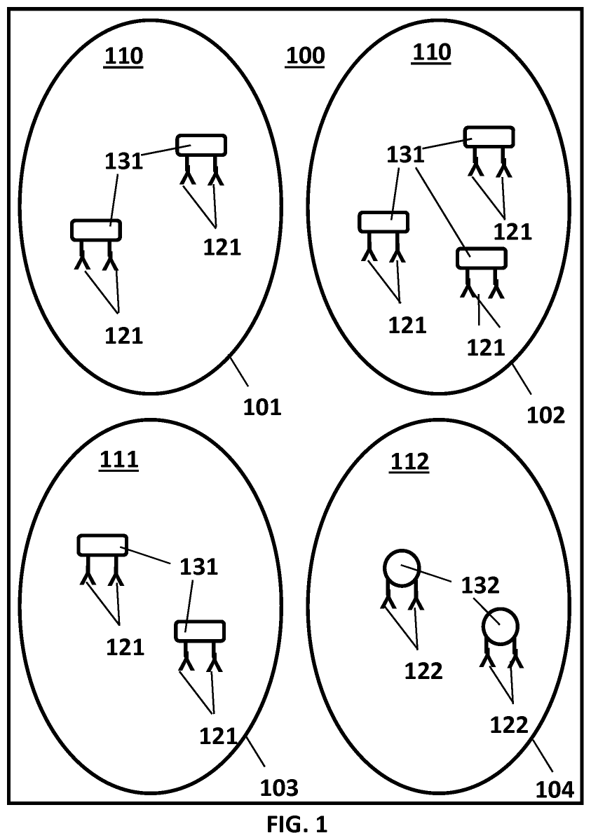

FIG. 1 depicts aspects of a probe library 100 as set forth herein. The probe library 100 comprises a plurality of vessels (101, 102, 103, 104), with each vessel comprising a probe composition. As shown, vessels 101, 102, 103, and 104 each comprise a unique probe composition (e.g., the probe composition of vessel 101 differs from the probe compositions of vessels 102, 103, and 104 with respect to at least one aspect of probe composition formulation). Vessels 101, 102, and 103 contain differing probe compositions for probes of a single species of affinity reagent 121 (i.e., all probes shown in vessels 101, 102, and 103 contain structurally identical affinity reagents 121). Further, the probes of the probe compositions in vessels 101, 102, and 103 all contain probes of identical architectures, with each probe comprising two affinity reagents 121 attached to a single first type of retaining component 131. The probe composition of vessel 102 differs from that of vessel 101 and 103 with respect to probe quantity or concentration, but has the same probe architecture and fluid composition (110) as vessel 101. The probe composition of vessel 103 differs from that of vessels 101 and 102 with respect to fluid composition (111), but has the same probe architecture as the probes of vessels 101 and 102, and the same or substantially similar probe quantity or concentration as vessel 101. The probe composition of vessel 104 differs from that of vessels 101, 102, and 103 with respect to probe architecture, probe binding specificity, and fluid composition. The probes of vessel 104 comprise a second type of affinity reagent 122, with each probe comprising two affinity reagents 122 attached to a single second type of retaining component 132. The second type of retaining component 132 differs from the first type of retaining component 131. Further, the fluid composition 112 in vessel 104 differs from that of vessels 101, 102, and 103 (110, 110, and 111, respectively). The probe composition of vessel 104 may have a same or substantially similar probe quantity or concentration as that of vessels 101 and 103.

### Definitions

Terms used herein will be understood to take on their ordinary meaning in the relevant art unless specified otherwise. Several terms used herein and their meanings are set forth below.

In some embodiments set forth herein, the terms “address” or “site” can refer to a location in an array where a particular analyte (e.g., protein, peptide or unique identifier label) is present. An address can contain a single analyte, or it can contain a population of several analytes of the same species (i.e. an ensemble of the analytes). Alternatively, an address can include a population of different analytes. Addresses are typically discrete. The discrete addresses can be contiguous, or they can be separated by interstitial spaces. An array useful herein can have, for example, addresses that are separated by less than 100 microns, 10 microns, 1 micron, 100 nm, 10 nm or less. Alternatively or additionally, an array can have addresses that are separated by at least 10 nm, 100 nm, 1 micron, 10 microns, or 100 microns. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

In some embodiments set forth herein, the term “affinity reagent” can refer to a molecule or other substance that is capable of specifically or reproducibly binding to an analyte (e.g., protein). An affinity reagent can be larger than, smaller than or the same size as the analyte. An affinity reagent may form a reversible or irreversible bond with an analyte. An affinity reagent may bind with an analyte in a covalent or non-covalent manner. Affinity reagents may include reactive affinity reagents, catalytic affinity reagents (e.g., kinases, proteases, etc.) or non-reactive affinity reagents (e.g., antibodies or fragments thereof). An affinity reagent can be non-reactive and non-catalytic, thereby not permanently altering the chemical structure of an analyte to which it binds. Affinity reagents that can be particularly useful for binding to proteins include, but are not limited to, antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

In some embodiments set forth herein, the term “array” can refer to a population of analytes (e.g., proteins) that are associated with unique identifiers such that the analytes can be distinguished from each other. A unique identifier can be, for example, a solid support (e.g., particle or bead), address on a solid support, tag, label (e.g., luminophore), or barcode (e.g., nucleic acid barcode) that is associated with an analyte and that is distinct from other identifiers in the array. Analytes can be associated with unique identifiers by attachment, for example, via covalent bonds or non-covalent bonds (e.g., ionic bond, hydrogen bond, van der Waals forces, electrostatics etc.). An array can include different analytes that are each attached to different unique identifiers. An array can include different unique identifiers that are attached to the same or similar analytes. An array can include separate solid supports or separate addresses that each bear a different analyte, wherein the different analytes can be identified according to the locations of the solid supports or addresses.

In some embodiments set forth herein, the term “attached” can refer to the state of two things being joined, fastened, adhered, connected or bound to each other. Attachment can be covalent or non-covalent. For example, a particle can be attached to a protein by a covalent or non-covalent bond. A covalent bond is characterized by the sharing of pairs of electrons between atoms. A non-covalent bond is a chemical bond that does not involve the sharing of pairs of electrons and can include, for example, hydrogen bonds, ionic bonds, van der Waals forces, hydrophilic interactions, adhesion, adsorption, and hydrophobic interactions.

In some embodiments set forth herein, the term “binding affinity” or “affinity” can refer to the strength or extent of binding between an affinity reagent and a binding partner. In some cases, the binding affinity of an affinity reagent for a binding partner may be vanishingly small or effectively zero. A binding affinity of an affinity reagent for a binding partner may be qualified as being a “high affinity,” “medium affinity,” or “low affinity.” A binding affinity of an affinity reagent for a binding partner, affinity target, or target moiety may be quantified as being “high affinity” if the interaction has a dissociation constant of less than about 100 nM, “medium affinity” if the interaction has a dissociation constant between about 100 nM and 1 mM, and “low affinity” if the interaction has a dissociation constant of greater than about 1 mM. Binding affinity can be described in terms known in the art of biochemistry such as equilibrium dissociation constant (KD), equilibrium association constant (KA), association rate constant (kon), dissociation rate constant (koff) and the like. See, for example, Segel, Enzyme Kinetics John Wiley and Sons, New York (1975), which is incorporated herein by reference in its entirety.

In some embodiments set forth herein, the term “binding probability” can refer to the probability that an affinity reagent or probe may be observed to interact with an analyte, for example, within a given binding context. A binding probability may be expressed as a discrete number (e.g., 0.4 or 40%) a matrix of discrete numbers, or as a mathematical model (e.g., a theoretical or empirical model). A binding probability may include one or more factors, including binding specificity, likelihood of locating a target epitope, or the likelihood of binding for a sufficient time to detect a binding interaction.

In some embodiments set forth herein, the term “binding profile” can refer to a plurality of binding outcomes for a protein or other analyte. The binding outcomes can be obtained from independent binding observations, for example, independent binding outcomes can be acquired using different affinity reagents, respectively. Alternatively, the binding outcomes can be generated in silico, for example, being derived from a modification of an empirically obtained binding outcome. A binding profile can include empirical measurement outcomes, candidate measurement outcomes, calculated measurement outcomes, theoretical measurement outcomes or a combination thereof. A binding profile can exclude one or more of empirical measurement outcomes, candidate measurement outcomes, calculated measurement outcomes, or theoretical measurement outcomes or putative measurement outcomes. A binding profile can include a vector of binding outcomes.

In some embodiments set forth herein, the term “binding specificity” can refer to the tendency of a binding reagent to preferentially interact with a given analyte relative to other analytes. A binding reagent may have a calculated, observed, known, or predicted binding specificity for a given analyte. Binding specificity may refer to selectivity for a single analyte in a given sample relative to one, some or all other analytes in the sample. Moreover, binding specificity may refer to selectivity for a subset of analytes in a given sample relative to at least one other analyte in the sample.

The term “comprising” is intended herein to be open-ended, including not only the recited elements, but further encompassing any additional elements.

In some embodiments set forth herein, the term “each,” when used in reference to a collection of items, is intended to identify an individual item in the collection but does not necessarily refer to every item in the collection. Exceptions can occur if explicit disclosure or context clearly dictates otherwise.

In some embodiments set forth herein, the term “epitope” can refer to an affinity target within a protein, polypeptide or other analyte. Epitopes may include amino acid sequences that are sequentially adjacent in the primary structure of a protein. Epitopes may include amino acids that are structurally adjacent in the secondary, tertiary or quaternary structure of a protein despite being non-adjacent in the primary sequence of the protein. An epitope can be, or can include, a moiety of protein that arises due to a post-translational modification, such as a phosphate, phosphotyrosine, phosphoserine, phosphothreonine, or phosphohistidine. An epitope can optionally be recognized by or bound to an antibody. However, an epitope need not necessarily be recognized by any antibody, for example, instead being recognized by an aptamer, mini-protein or other affinity reagent. An epitope can optionally bind an antibody to elicit an immune response. However, an epitope need not necessarily participate in, nor be capable of, eliciting an immune response.

In some embodiments set forth herein, the term “exogenous,” when used in reference to a moiety of a molecule, can mean the moiety is not present in a natural analog of the molecule. For example, an exogenous label of an amino acid is a label that is not present on a naturally occurring amino acid. Similarly, an exogenous label that is present on an antibody is not found on the antibody in its native milieu.

In some embodiments set forth herein, the term “fluid-phase,” when used in reference to a molecule, can mean the molecule is in a state wherein it is mobile in a fluid, for example, being capable of diffusing through the fluid.

In some embodiments set forth herein, the terms “group” and “moiety” are intended to be synonymous when used in reference to the structure of a molecule. The terms can refer to a component or part of the molecule. The terms do not necessarily denote the relative size of the component or part compared to the rest of the molecule, unless indicated otherwise.

In some embodiments set forth herein, the term “immobilized,” when used in reference to a molecule that is in contact with a fluid phase, can refer to the molecule being prevented from diffusing in the fluid phase. For example, immobilization can occur due to the molecule being confined at, or attached to, a solid phase. Immobilization can be temporary (e.g., for the duration of one or more steps of a method set forth herein) or permanent. Immobilization can be reversible or irreversible under conditions utilized for a method, system or composition set forth herein.

In some embodiments set forth herein, the terms “label” and “detectable label can refer to a molecule or moiety that provides a detectable characteristic. The detectable characteristic can be, for example, an optical signal such as absorbance of radiation, luminescence emission, luminescence lifetime, luminescence polarization, fluorescence emission, fluorescence lifetime, fluorescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like. A label may produce a signal that is detectable in real-time (e.g., fluorescence, luminescence, radioactivity). A label may produce a signal that is detected off-line (e.g., a nucleic acid barcode) or in a time-resolved manner (e.g., time-resolved fluorescence). A label may produce a signal with a characteristic frequency, intensity, polarity, duration, wavelength, sequence, or fingerprint.

In some embodiments set forth herein, the terms “linker” or “linking moiety” can refer to a moiety that connects two objects to each other. One or both objects can be a molecule, solid support, address, particle or bead. Both objects can be moieties of a molecule, solid support, address, particle or bead. The term can also refer to an atom, moiety or molecule that is configured to react with two objects to form a moiety that connects the two objects. The connection of a linker to one or both objects can be a covalent bond or non-covalent bond. A linker may be configured to provide a chemical or mechanical property to the moiety connecting two objects, such as hydrophobicity, hydrophilicity, electrical charge, polarity, rigidity, or flexibility. A linker may comprise two or more functional groups that facilitate coupling of the linker to the first and second objects. A linker may include a polyfunctional linker such as a homobifunctional linker, heterobifunctional linker, homopolyfunctional linker, or heteropolyfunctional linker. Exemplary compositions for linkers can include, but are not limited to, a polyethylene glycol (PEG), polyethylene oxide (PEO), amino acid, protein, nucleotide, nucleic acid, nucleic acid origami, dendrimer, protein nucleic acid (PNA), polysaccharide, carbon, nitrogen, oxygen, ether, sulfur, or disulfide. A linker can be a bead or particle such as a structured nucleic acid particle.

In some embodiments set forth herein, the term “measurement outcome” can refer to information resulting from observation, simulation or examination of a process. For example, the measurement outcome for contacting an affinity reagent with an analyte can be referred to as a “binding outcome.” A measurement outcome can be positive or negative. For example, observation of binding is a positive binding outcome and observation of non-binding is a negative binding outcome. A measurement outcome can be a null outcome in the event a positive or negative outcome is not apparent from a given measurement. An “empirical” measurement outcome includes information based on observation of a signal from an analytical technique. A “putative” measurement outcome includes information based on theoretical or a priori evaluation of an analytical technique or analytes. A “candidate” measurement outcome includes an empirical or putative measurement outcome for a candidate analyte (e.g., for a candidate protein) that is known or suspected of being present in a sample or assay. A measurement outcome can be represented in binary terms, such as a zero (0) for a negative binding outcome and a one (1) for a positive binding outcome. In some cases a ternary representation can be used, for example, when zero (0) represents a negative binding outcome, one (1) represents a positive binding outcome, and two (2) represents a null outcome. It is also possible to use continuous or analog values, as opposed to integers or discrete values, to represent different measurement outcomes.

In some embodiments set forth herein, the term “nucleic acid origami” can refer to a nucleic acid construct having an engineered tertiary or quaternary structure. A nucleic acid origami may include DNA, RNA, PNA, modified or non-natural nucleic acids, or combinations thereof. A nucleic acid origami may include a plurality of oligonucleotides that hybridize via sequence complementarity to produce the engineered structuring of the origami. A nucleic acid origami may include sections of single-stranded or double-stranded nucleic acid, or combinations thereof. Exemplary nucleic acid origami structures may include nanotubes, nanowires, cages, tiles, nanospheres, blocks, and combinations thereof. A nucleic acid origami can optionally include a relatively long scaffold nucleic acid to which multiple smaller nucleic acids hybridize, thereby creating folds and bends in the scaffold that produce an engineered structure. The scaffold nucleic acid can be circular or linear. The scaffold nucleic acid can be single stranded but for hybridization to the smaller nucleic acids. A smaller nucleic acid (sometimes referred to as a “staple”) can hybridize to two regions of the scaffold, wherein the two regions of the scaffold are separated by an intervening region that does not hybridize to the smaller nucleic acid.

In some embodiments set forth herein, the term “nucleic acid tag” can refer to a nucleic acid molecule or sequence that is encoded with information that uniquely identifies an object with which it is associated. A nucleic acid tag can be associated with an object via a connection. The connection can be physical, including for example, attachment, colocalization, diffusional contact or the like. Non-physical connections can include, for example, knowledge of a past interaction, knowledge of a shared characteristic, knowledge of common manipulations, knowledge of origin or the like. The nucleic acid tag can be, for example, DNA, RNA or analogs thereof. The length of the tag sequence can be at least about 5, 8, 10, 15, 20, 25, 30, 40, 50, 75, 100 or more nucleotides. Alternatively or additionally, the length of the tag sequence can be at most about 100, 75, 50, 40, 30, 25, 20, 15, 10, 8, 5 or fewer nucleotides.

In some embodiments set forth herein, the term “post-translational modification” can refer to a change to the chemical composition of a protein compared to the chemical composition encoded by the gene for the protein. Exemplary changes include those that alter the presence, absence or relative arrangement of different regions of amino acid sequence (e.g., splicing variants, or protein processing variants of a single gene), or due to presence or absence of different moieties on particular amino acids (e.g., post-translationally modified variants of a single gene). A post-translational modification can be derived from an in vivo process or in vitro process. A post-translational modification can be derived from a natural process or a synthetic process. Exemplary post-translational modifications include those classified by the PSI-MOD ontology. See Smith, L. M. et al. Nat. Methods, 2013, 10, 186-187.

In some embodiments set forth herein, the term “primary,” when used in reference to an affinity reagent, can refer to the affinity reagent having a binding specificity for an analyte that is or may be present in a plurality of analytes. In some embodiments set forth herein, the term “secondary,” when used in reference to an affinity reagent, can refer to the affinity reagent having a binding specificity for another affinity reagent. Accordingly, a primary affinity reagent may be directly bound to an analyte, whereas a secondary affinity reagent may only be bound to an analyte by an intermediary primary affinity reagent to which the secondary affinity reagent is bound.

In some embodiments set forth herein, the term “probe” can refer to a molecule or particle that is capable of forming a binding interaction with a binding target. A probe can comprise a single affinity reagent or a plurality thereof. A probe may further comprise additional components, such as a linking moiety, a detectable label, or a tether strand. In some embodiments set forth herein, a “detectable probe” can refer to a probe composition that comprises one or more detectable labels. A detectable probe may comprise a detectable label that facilitates detection of a complex formed by the binding of the probe to a binding target of the probe.

In some embodiments set forth herein, the terms “promiscuous” or “multi-affinity,” when used in reference to a reagent such as a probe, binding reagent, or affinity reagent, can mean that the reagent is known or suspected to react with a variety of different analytes in a given sample. For example, an affinity reagent that is known or suspected to recognize a variety of different analytes (e.g., a variety of proteins having different primary sequences) is promiscuous. A reagent may be promiscuous if it binds to differing analytes that share a common epitope or any member of a family of epitopes. A promiscuous reagent may be known or suspected of having high reactivity with one or more of the different analytes with which it reacts. For example, a promiscuous affinity reagent may have high affinity for one or more of the different analytes that it recognizes. A promiscuous reagent may be composed of a single species of reagent, such as a single affinity reagent, or a promiscuous reagent may be composed of two or more different species of reagent. For example, a promiscuous affinity reagent may be composed of a single species of antibody that recognizes a variety of different proteins in a sample, or the promiscuous affinity reagent may be composed of a pool containing several different antibody species that collectively recognize the variety of different proteins in the sample. A promiscuous reagent may have a binding specificity for two or more different proteins as determined by primary amino acid sequence (i.e., the two or more different proteins are not isoforms of the same protein). A promiscuous reagent may have a binding specificity for an epitope of about 2, 3, 4, 5, 6, or 7 amino acids in total length (e.g., 2 to 7 consecutive amino acids, 2 to 7 contiguous amino acids). A promiscuous reagent may have a binding specificity for an epitope that is common to two or more differing proteins. A promiscuous reagent may bind to multiple forms of a single protein such as multiple proteoforms and/or isoforms of that protein. In some embodiments set forth herein, the term “single-affinity,” when used in reference to a reagent such as a probe, binding reagent, or affinity reagent, can mean that the reagent is known or suspected to react with only one analyte of a variety of different analytes in a given sample. A single-affinity reagent may be composed of a single species of reagent, such as a single affinity reagent, or a single-affinity reagent may be composed of two or more different species of reagent. For example, a single-affinity binding reagent may be composed of a single species of antibody that recognizes only one protein of a variety of different proteins in a sample, or the single-affinity binding reagent may be composed of a pool containing several different antibody species that each individually recognize the same single protein of the variety of different proteins in the sample. A single-affinity reagent may bind to only one form of a single protein such as a single proteoform or single isoform of that protein. A single-affinity reagent may have a binding specificity for a single protein, as determined by primary amino acid sequence.

In some embodiments set forth herein, the term “protein” can refer to a molecule comprising two or more amino acids joined by a peptide bond. A protein may also be referred to as a polypeptide, oligopeptide or peptide. A protein can be a naturally-occurring molecule, or synthetic molecule. A protein may include one or more non-natural amino acids, modified amino acids, or non-amino acid linkers. A protein may contain D-amino acid enantiomers, L-amino acid enantiomers or both. Amino acids of a protein may be modified naturally or synthetically, such as by post-translational modifications. In some circumstances, different proteins may be distinguished from each other based on different genes from which they are expressed in an organism, different primary sequence length or different primary sequence composition.

Proteins expressed from the same gene may nonetheless be different proteoforms, for example, being distinguished based on non-identical length, non-identical amino acid sequence or non-identical post-translational modifications. Different proteins can be distinguished based on one or both of gene of origin and proteoform state.

In some embodiments set forth herein, the term “single,” when used in reference to an object such as an analyte, can mean that the object is individually manipulated or distinguished from other objects. A single analyte can be a single molecule (e.g., single protein), a single complex of two or more molecules (e.g., a multimeric protein having two or more separable subunits, a single protein attached to a structured nucleic acid particle or a single protein attached to an affinity reagent), a single particle, or the like. Reference herein to a “single analyte” in the context of a composition, system or method herein does not necessarily exclude application of the composition, system or method to multiple single analytes that are manipulated or distinguished individually, unless indicated contextually or explicitly to the contrary.

In some embodiments set forth herein, the term “single-analyte resolution” can refer to the detection of or ability to detect an analyte on an individual basis, for example, as distinguished from its nearest neighbor in an array.

In some embodiments set forth herein, the term “solid support” can refer to a substrate that is insoluble in aqueous liquid. Optionally, the substrate can be rigid. The substrate can be non-porous or porous. The substrate can optionally be capable of taking up a liquid (e.g., due to porosity) but will typically, but not necessarily, be sufficiently rigid that the substrate does not swell substantially when taking up the liquid and does not contract substantially when the liquid is removed by drying. A nonporous solid support is generally impermeable to liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In particular configurations, a flow cell contains the solid support such that fluids introduced to the flow cell can interact with a surface of the solid support to which one or more components of a binding event (or other reaction) is attached.

In some embodiments set forth herein, the term “structured nucleic acid particle” or “SNAP” can refer to a single- or multi-chain polynucleotide molecule having a compacted three-dimensional structure. The compacted three-dimensional structure can optionally be characterized in terms of hydrodynamic radius or Stoke's radius of the SNAP relative to a random coil or other non-structured state for a nucleic acid having the same sequence length as the SNAP. The compacted three-dimensional structure can optionally be characterized with regard to tertiary structure. For example, a SNAP can be configured to have an increased number of internal binding interactions between regions of a polynucleotide strand, less distance between the regions, increased number of bends in the strand, and/or more acute bends in the strand, as compared to a nucleic acid molecule of similar length in a random coil or other non-structured state. Alternatively or additionally, the compacted three-dimensional structure can optionally be characterized with regard to tertiary or quaternary structure. For example, a SNAP can be configured to have an increased number of interactions between polynucleotide strands or less distance between the strands, as compared to a nucleic acid molecule of similar length in a random coil or other non-structured state. In some configurations, the secondary structure of a SNAP can be configured to be more dense than a nucleic acid molecule of similar length in a random coil or other non-structured state. A SNAP may contain DNA, RNA, PNA, modified or non-natural nucleic acids, or combinations thereof. A SNAP may include a plurality of oligonucleotides that hybridize to form the SNAP structure. The plurality of oligonucleotides in a SNAP may include oligonucleotides that are attached to other molecules (e.g., probes, analytes such as proteins, reactive moieties, or detectable labels) or are configured to be attached to other molecules (e.g., by functional groups). A SNAP may include engineered or rationally designed structures. Exemplary SNAPs include nucleic acid origami and nucleic acid nanoballs.

In some embodiments set forth herein, the term “type,” when used in reference to a subset of analytes, can refer to a characteristic that is shared by the analytes in the subset and that distinguishes the analytes in the subset from analytes that are not in the subset. The characteristic can be any of a variety of characteristics known for the analytes. Any of a variety of analytes can be categorized by type, including for example, proteins. Exemplary characteristics that can be used to categorize proteins by type include, but are not limited to, amino acid composition, full length amino acid sequence, proteoform, presence or absence of an amino acid sequence motif, number of amino acids present (i.e. sequence length), molecular weight, presence or absence of a particular epitope, presence or absence of epitope(s) recognized by a particular affinity reagent, probability of binding a particular affinity reagent, presence or absence of a post-translational modification, enzymatic activity, affinity for binding a particular protein or protein motif, or the like.

In some embodiments set forth herein, the term “unique identifier” can refer to a moiety, object or substance that is associated with an analyte and that is distinct from other identifiers, throughout one or more steps of a process. The moiety, object or substance can be, for example, a solid support such as a particle or bead; a location on a solid support; a spatial address in an array; a tag; a label such as a luminophore; a molecular barcode such as a nucleic acid having a unique nucleotide sequence or a protein having a unique amino acid sequence; or an encoded device such as a radiofrequency identification (RFID) chip, electronically encoded device, magnetically encoded device or optically encoded device. The process in which a unique identifier is used can be an analytical process, such as a method for detecting, identifying, characterizing or quantifying an analyte; a separation process in which at least on analyte is separated from other analytes; or a synthetic process in which an analyte is modified or produced. The unique identifier can be associated with an analyte via immobilization. For example, a unique identifier can be covalently or non-covalently (e.g., ionic bond, hydrogen bond, van der Waals forces etc.) attached to an analyte. A unique identifier can be exogenous to an associated analyte, for example, being synthetically attached to the associated analyte. Alternatively, a unique identifier can be endogenous to the analyte, for example, being attached or associated with the analyte in the native milieu of the analyte.

In some embodiments set forth herein, the term “unique identifier label” can refer to a unique identifier that is a particle, molecule or moiety that provides a detectable characteristic. The detectable characteristic can be, for example, an optical signal such as absorbance of radiation, luminescence (e.g., fluorescence) emission, luminescence lifetime, luminescence polarization, or the like; Rayleigh and/or Mie scattering; binding affinity for a ligand or receptor; magnetic properties; electrical properties; charge; mass; radioactivity or the like. Exemplary labels include, without limitation, a fluorophore, luminophore, chromophore, nanoparticle (e.g., gold, silver, carbon nanotubes), heavy atoms, radioactive isotope, mass label, charge label, spin label, receptor, ligand, or the like.

In some embodiments set forth herein, the term “vessel” can refer to an enclosure that contains a substance. The enclosure can be permanent or temporary with respect to the timeframe of a method set forth herein or with respect to one or more steps of a method set forth herein. Exemplary vessels include, but are not limited to, a well (e.g., in a multiwell plate or array of wells), test tube, channel, tubing, pipe, flow cell, bottle, vesicle, droplet that is immiscible in a surrounding fluid, or the like. A vessel can be entirely sealed to prevent fluid communication from inside to outside, and vice versa. Alternatively, a vessel can include one or more ingress or egress to allow fluid communication between the inside and outside of the vessel. A vessel can be made from multiple materials, for example, including a well in a solid support that is covered by a seal such as a wax or fluid that is immiscible with fluid in the well.

The embodiments set forth below and recited in the claims can be understood in view of the above definitions.

### Probe Libraries

In an aspect, provided herein is a probe library comprising a first probe composition and a second probe composition, the first probe composition differing from the second probe composition with respect to probe architecture, probe concentration, or fluid composition. In another aspect, further provided herein is a probe library comprising a first probe composition and a second probe composition, the first probe composition differing from the second probe composition with respect to probe concentration. The probe library may be provided as a kit, for example a kit utilized to perform a probe-based assay in a system set forth herein. In accordance with some embodiments set forth herein, the term “probe composition” can refer to a plurality of detectable probes co-localized within a single vessel, as set forth herein. A probe composition can be provided in a fluidic phase or in a solid phase. A probe library may comprise at least about 2, 5, 10, 20, 25, 30, 40, 50, 75, 100, 125, 150, 200, 250, 300, 350, 400, 450, 500, 1000, 2000, 5000, 10000, or more than 10000 different probe compositions. Alternatively or additionally, a probe library may comprise no more than about 10000, 5000, 2000, 1000, 500, 450, 400, 350, 300, 250, 200, 150, 125, 100, 75, 50, 40, 30, 25, 20, 10, 5, or less than 5 different probe compositions.

A probe library may comprise a plurality of probe compositions, in which each individual probe composition of the plurality of probe compositions is physically or fluidically isolated from each other probe composition of the plurality of probe compositions. A probe library may comprise a plurality of vessels, in which the plurality of vessels is contiguous or conjoined. In some configurations, a probe library may comprise a plurality of vessels, in which each vessel of the plurality of vessels comprises a different probe composition. A probe library may comprise a plurality of vessels, in which each vessel of the plurality of vessels is fluidically isolated from each other vessel of the plurality of vessels (i.e., fluid cannot passively transfer from one vessel to another vessel). A vessel comprising a probe composition may be sealed, for example by a sealing material (e.g., metal foil, polymer film, wax, adhesive, plastic, etc.). A vessel may be sealed if fluid cannot be transferred into or out of the vessel. A probe library comprising a sealed vessel may be provided to a system that is configured to puncture, break, or otherwise disrupt the seal of the vessel, thereby facilitating removal of a probe composition from the vessel.

A probe library may comprise at least about 10, 20, 30, 40, 50, 75, 100, 150, 200, 250, 300, 400, 500, 1000, 1200, 1500, 2000, 5000, 10000, or more than 10000 vessels. Alternatively or additionally, a probe library may comprise no more than about 10000, 5000, 2000, 1500, 1200, 1000, 500, 400, 300, 250, 200, 150, 100, 75, 50, 40, 30, 20, 10, or less than 10 vessels.

A vessel of a probe library can have a volume of at least about 1 microliter (μl), 10 μl, 20 μl, 50 μl, 100 μl, 200 μl, 300 μl, 400 μl, 500 μl, 1 milliliter (ml), 2 ml, 5 ml, 10 ml, or more than 10 ml. Alternatively or additionally, a vessel of a probe library can have a volume of no more than about 10 ml, 5 ml, 2 ml, 1 ml, 500 μl, 400 μl, 300 μl, 200 μl, 100 μl, 50 μl, 20 μl, 10 μl, 10 μl, or less than 1 μl.

A probe library may be provided with a plurality of probe compositions such that each probe composition of the plurality of probe compositions is substantially devoid of analytes of interest. A system set forth herein may comprise a fluidic system that provides fluidic communication between a probe composition of a probe library and a vessel comprising a plurality of analytes of interest. In some configurations, a probe library may include one or more vessels that contain analytes, such as control analytes or standard analytes. A vessel containing analytes may be substantially devoid of any probes, as set forth herein.

A plurality of vessels may be provided in a cartridge or plate format. A cartridge or plate may comprise a substrate that joins individual vessels of a plurality of vessels into a single body. For example, a kit may comprise a 96-well, 192-well, 384-well, or 1536-well plate, in which individual wells of the plate are vessels, each vessel containing a probe composition. The vessels of a plate or cartridge may be formed from a rigid material. Accordingly, the vessels may have a substantially invariant maximum volume. Alternatively, a plurality of vessels may be provided in a pouch or compartment. Vessels of a pouch or compartment may be formed from a non-rigid or flexible material. For example, a probe composition may be provided in a blister pack format. Pouches or compartments can be provided in numerous formats, such as tapes, rolls, chains, and sheets. For example, a kit may comprise a roll of blister packs, each blister pack of the roll containing a probe composition.

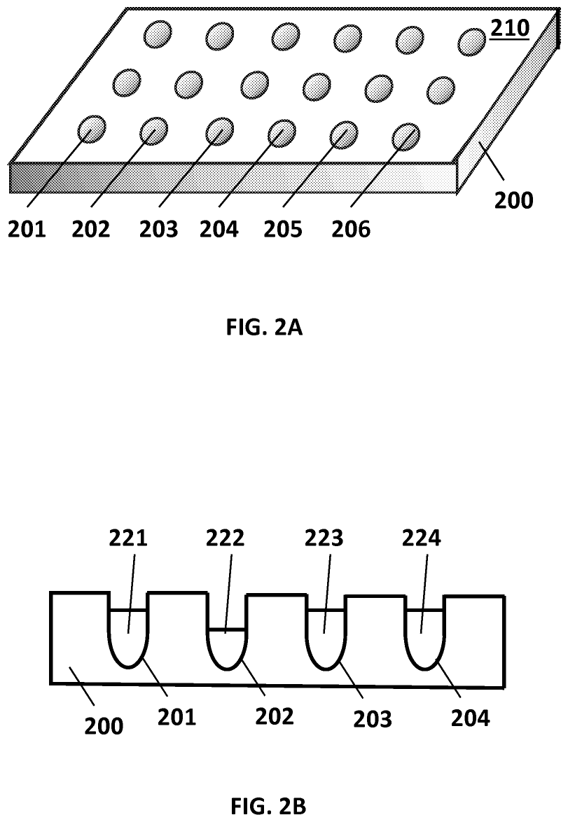

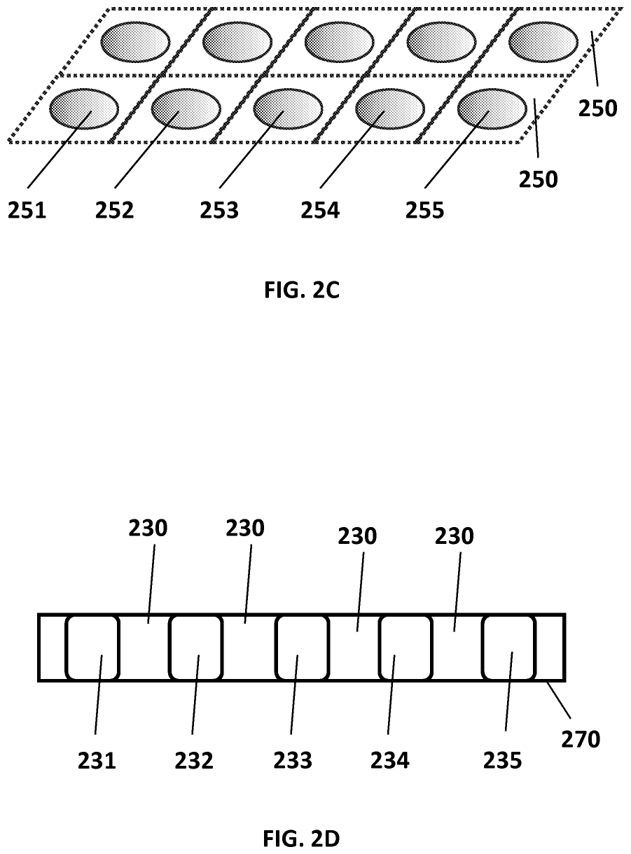

FIGS. 2A-2D illustrate some forms of kits with which a probe library can be provided. FIG. 2A depicts an isometric view of a cartridge format for providing a probe library, in which the cartridge 200 comprises a molded substrate with a plurality of separated or divided vessels (e.g., 201, 202, 203, 204, 205, 206). In some configurations, the cartridge may comprise a surface 210 to which a seal is applied across vessel openings, thereby preventing transfer of fluid or other contaminants into or out of the vessels. FIG. 2B depicts a cross-sectional view of a portion of the cartridge of FIG. 2A. Vessels 201, 202, 203, and 204 contain fluidic media (221, 222, 223, and 224, respectively). The volume of fluidic media provided to vessels 201, 203, and 204 is substantially the same, while vessel 202 contains a smaller volume of fluid than vessels 201, 203, and 204. The reduced fluid volume could be utilized to provide a larger concentration of probes (e.g., if each vessel contains a similar quantity of probes), or could be utilized to provide a smaller quantity of probes (e.g., if the concentration of each fluidic media is similar or substantially the same).

FIG. 2C depicts a blister pack format for providing a probe library, in which individual vessels are formed from pouches or compartments (e.g., 251, 252, 253, 254, 255) of a substrate 250 (e.g., two laminated materials). The blister pack of FIG. 2C is depicted in a sheet format of M columns by N rows, where M×N is the total number of pouches or compartments provided in the blister pack (2×N as shown in FIG. 2C, but readily adaptable to 1×N, 3×N, 4×N, 5×N, etc.). The blister pack of FIG. 2C is shown in a sheet format, but could be rolled or folded to provide a more compact form factor. Creasing or perforation of the substrate 250 between pouches or compartments may facilitate rolled or folded configurations of the blister pack.

FIG. 2D depicts a cross-sectional view of an alternative configuration of a probe library kit within a single vessel 270, such as a tube or capillary. The vessel 270 comprises a plurality of volumes of fluid (231, 232, 233, 234, 235) containing probe compositions. Each fluid containing a probe composition is separated from each other fluid containing a probe composition by a volume of separating fluid 230 (e.g., air; an immiscible liquid, etc.). The separating fluid 230 can prevent contact or mixing between differing probe compositions. The configuration of FIG. 2D may be provided as a storage kit for a probe library, or may be formed by transfer of probe compositions from another probe library kit configuration set forth herein.

In some configurations, a probe library may comprise a plurality of vessels, in which a vessel of the plurality of vessels comprises a fluidic medium. In particular configurations, a probe library may comprise a plurality of vessels, in which each vessel of the plurality of vessels comprises a fluidic medium. In some configurations, a probe library may comprise a plurality of vessels, in which a vessel of the plurality of vessels is substantially devoid of a fluidic medium. A vessel that is substantially devoid of a fluidic medium may comprise a solid phase containing a probe composition (e.g., a lyophilized probe composition). In particular configurations, a probe library may comprise a plurality of vessels, in which each vessel of the plurality of vessels is substantially devoid of a fluidic medium.

A vessel may comprise a probe composition comprising a fluidic medium, in which the fluidic medium has a volume of at least about 1 microliter (μl), 10 μl, 20 μl, 50 μl, 100 μl, 200 μl, 300 μl, 400 μl, 500 μl, 1 milliliter (ml), 2 ml, 5 ml, 10 ml, or more than 10 ml. Alternatively or additionally, a vessel may comprise a probe composition comprising a fluidic medium, in which the fluidic medium has a volume of no more than about 10 ml, 5 ml, 2 ml, 1 ml, 500 μl, 400 μl, 300 μl, 200 μl, 100 μl, 50 μl, 20 μl, 10 μl, 10 μl, or less than 1 μl.

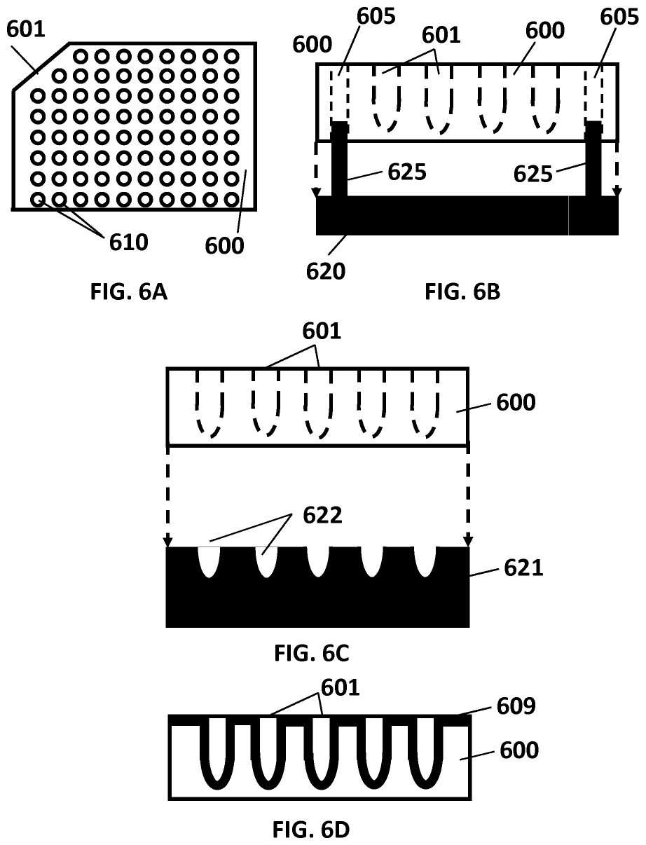

A probe library may be physically structured for placement in a system such as systems set forth herein for analyte characterization. The physical structure of a probe library may include structures that facilitate placement and/or attachment of the probe library to a retention structure of a system. A retention structure may be a caddy, platform, or holder that is configured to hold or maintain a probe library, as set forth herein. FIGS. 6A-6C illustrate non-exhaustive examples of probe library structures that can facilitate attachment of the probe library to a retention structure. FIG. 6A depicts a top-down view of a probe library cartridge 600 containing a plurality of wells 610 and further containing a reference edge 601 at a corner of the cartridge. The reference edge 601 produces an asymmetry or non-uniformity in the cartridge structure that may mate to a corresponding structure of a retention structure. FIG. 6B depicts a cross-sectional view of a probe library cartridge 600 containing wells 601 that open to the upper surface of the cartridge 600 and pass-through holes 605 that open to the bottom surface and optionally the top surface of the cartridge 600. The pass-through holes 605 can align to posts 625 of a retention structure 620, thereby orienting and attaching the cartridge 600 to the retention structure 620. FIG. 6C depicts a cross-sectional view of an alternative retention structure 621 that contains patterned depressions 622 in a top surface that align with the positions of wells 601 of a probe library cartridge 600. When the cartridge 600 is contacted to the retention structure 621 in the direction shown by the arrows, the wells 601 can mate to the depressions 622, thereby attaching the cartridge 600 to the retention structure 621. A probe library or a retention structure for a probe library can include any sort of suitable fastener or attachment system for securing and/or stabilizing the probe library with respect to the retention structure. Fasteners can include pins, screws, bolts, clips, clamps, and latches. A probe library or a retention structure for a probe library can include a structure that mates with a fastener, such as a divot, groove, hole, slot, post, or combinations thereof.

A retention structure that attaches to a probe library may be configured to regulate or maintain a condition of a probe library or a probe composition thereof. A retention structure can be configured to shake, oscillate, vibrate, or sonicate a probe library, thereby providing mechanical mixing to the probe library. In some cases, mechanical mixing may disrupt aggregation of probes or inhibit sedimentation of probes. A retention structure may include a heat transfer device (e.g., a Pelletier device, a heat exchanger, a liquid bath) that can heat and/or cool a probe library. A retention structure that directly contacts vessels of a probe library (e.g., 621 of FIG. 6C) may be useful for temperature regulation and/or mechanical mixing. A probe library may be provided a hollow space or void space surrounding its vessels that can be contacted with a circulating fluid, thereby facilitating temperature control via a fluid bath.

Materials for the fabrication of a probe library, as set forth herein, are not particularly limited. Plastics, glasses, and/or metals may be useful for probe libraries provided in a cartridge format. Probe libraries may be fabricated by the layering of multiple materials depending upon anticipated environmental conditions. For example, a probe library may incorporate a layer of a heat transfer medium, a gas-impermeable material, a liquid-impermeable material, an optically opaque material, and/or a reflective material. FIG. 6D illustrates a cross-sectional view of a probe library cartridge 600 comprising a plurality of wells 601 and a conformal layered material 609 that is conformed to the external surfaces of the wells 601 and portions of the body of the cartridge 600. This configuration could be useful for applying a heat transfer material (e.g., a metal or metal oxide) that facilitates heat transfer from a retention structure (e.g., 621 of FIG. 6C). This configuration could also be useful for applying a material that is opaque to ultraviolet or visible light for photo-protection of wells containing light-sensitive reagents.

For a probe library comprising a plurality of vessels, probe compositions may be provided to vessels of the plurality of vessels with a spatial ordering. For example, vessels may be ordered according to the expected order of usage of the associated probe compositions during an analytical assay, as set forth herein (e.g., first vessel's probe composition utilized first, second vessel's probe composition utilized second, etc.). In another example, contiguous or neighboring vessels containing probe compositions may be grouped according to a common property of the probe compositions (a common probe architecture, a common fluid composition, a common kinetic property, etc.). In some cases, contiguous or neighboring vessels containing probe compositions may be grouped according to a common assay procedure. For example, contiguous or neighboring vessels containing probe compositions may be grouped according to use of a common pre-conditioning agent for analytes or other binding targets of the probe compositions. In another example, contiguous or neighboring vessels containing probe compositions may be grouped according to use of a common probe dissociation medium for each of the probe compositions. For a probe library comprising a plurality of vessels, probe compositions may be provided to vessels of the plurality of vessels with no spatial ordering.

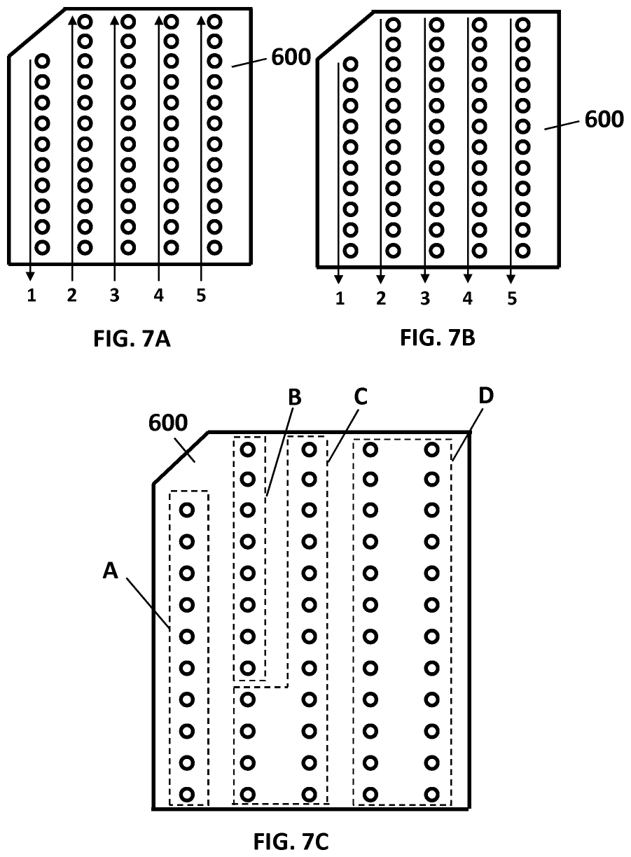

FIGS. 7A-7C depict top-down views of probe library cartridges 600. Numbered arrows indicate sequence of transfer of fluids from wells of the cartridge, for example by an automated pipetting system. FIG. 7A depicts an alternating transfer pattern, with fluid transfer beginning at the uppermost well of column 1, proceeding to the bottommost well of column 1, then moving to the bottommost well of column 2 before proceeding toward the uppermost well of column 2, etc. FIG. 7B depicts a unidirectional transfer pattern, with fluid transfer always proceeding from an uppermost well of each column to a bottommost well. Choice of fluid transfer pattern from a probe library may depend upon the configuration of a fluid transfer device and the particular configuration of an assay that utilizes the probe library.

FIG. 7C illustrates a probe library cartridge 600 with sets of wells grouped in dashed boxes A, B, C, and D. The sets of wells may be spatially grouped according to a common probe composition characteristic, as set forth herein. For example, wells in group A may each contain a different probe but a common fluid composition, and wells in group B may each contain probes having a common probe architecture. Sequencing of probes according to a shared characteristic may be preferable in some assays due to differing probe behaviors. For example, probes of group A may be optimally dissociated from analytes by a particular dissociation medium, while probes of group B may be optimally dissociated from analytes by a differing dissociation medium from the probes of group A.

It will be recognized that probe compositions sharing common characteristics or assay procedures can also be spatially distributed in a non-contiguous or non-neighboring manner. In some cases, a probe library may further comprise an information tag, such as a serial number, QR code, bar code, or RFID tag, that contains information regarding the spatial ordering of probe compositions of a probe library. A system, as set forth herein, may be configured to obtain information from an information tag of a probe library and based upon the obtained information, identify the spatial location (e.g., a vessel of a plurality of vessels) of a probe composition of a plurality of probe compositions of the probe library. In some cases, a system may be configured to access a computer, processor, or server that contains information from an information tag and information regarding the spatial ordering of probe compositions. Accordingly, the present disclosure provides two unique probe libraries, in which the two unique probe libraries each comprise a same plurality of probe compositions, in which the two unique probe libraries have a differing spatial order of the plurality of probe compositions, and in which the two unique probe libraries separately comprise an information tag that facilitates determination of the spatial ordering of the plurality of probe compositions by a system, as set forth herein.

The present disclosure provides probe libraries containing ordered or arranged sets of probe compositions. The ordering or arrangement may correspond to a sequence of probe compositions utilized in a method set forth herein. A probe library may contain two or more consecutive or neighboring vessels containing a same type of probe compositions (e.g., two or more consecutive or neighboring single-affinity probe compositions, two or more consecutive or neighboring multi-affinity probe compositions, two or more probe compositions having a common probe architecture, two or more consecutive probe compositions having a common fluid composition, two or more consecutive probe compositions having a same probe concentration, etc.). A probe library may contain a first vessel containing a probe composition, in which no neighboring or adjacent vessels contain a probe composition sharing a characteristic with the probe composition of the first vessel. For example, a probe library may contain a mixture of multi-affinity probe compositions and single-affinity probe compositions, in which a row of the probe library has a sequence of probe compositions of: multi-affinity, multi-affinity, single-affinity, multi-affinity, multi-affinity, etc. Probe compositions sharing a common characteristic may be sequenced or ordered in a probe library in a regular interval, such as once every N vessels (e.g., a single-affinity probe composition once every about 10, 20, 30, 40, 50 or more vessels).

A probe library may comprise a plurality of vessels, in which one or more of the vessels does not comprise a probe composition. A probe library may be provided with one or more vessels comprising fluidic media containing reagents such as blocking agents (e.g., bovine serum albumin, PF-127, polyvinyl pyrrolidone, etc.), analyte conditioning agents (e.g., denaturants, chaotropes, surfactants, etc.), or probe removal agents. A probe library may be provided with two or more vessels comprising differing compositions of a common type of assay reagent (e.g., differing blocking agents, differing analyte conditioning agents, differing probe dissociation agents, differing excipient agents). For example, a probe library may be provided with a more stringent probe dissociation agent that is used with one or more probe compositions of the probe library, and may be further provided with a less stringent probe dissociation agent that is used with one or more other probe compositions of the probe library. In some cases, a probe library may be provided as a probe kit, in which a reagent is provided in a same type of vessel as a probe composition (e.g., a well of a multiwell plate). In other cases, a probe library may be provided as a probe kit, in which a reagent is provided in a separate vessel (e.g., a bottle, an ampule, etc.) from a plurality of vessels comprising probe compositions).

A probe composition may comprise a plurality of probes, in which each probe of the plurality of probes comprises an affinity reagent that is structurally identical to the affinity reagents of each other probe of the plurality of probes. Accordingly, each probe of the plurality of probes of the probe composition can have an identical binding specificity to each other probe of the plurality of probes. The present disclosure further provides multiplexed probe compositions, in which the probe composition comprises two or more sets of probes, in which each probe of any given set of probes is structurally identical to each other probe of the set of probes, and in which each set of probes differs from each other set of probes (e.g., with respect to affinity reagent structure, with respect to probe architecture, with respect to binding specificity, etc.). For example, a multiplexed probe composition may comprise a first set of probes and a second set of probes, in which the probes of the first set of probes each comprise first detectable labels, in which the probes of the second set of probes each comprise second detectable labels, and in which the first detectable labels differ from the second detectable labels (e.g., with respect to excitation or emission wavelength, etc.). In another example, a multiplexed probe composition may comprise a first set of probes and a second set of probes, in which the probes of the first set of probes each comprise a first affinity reagent, in which the probes of the second set of probes each comprise a second affinity reagent, in which the first affinity reagents differ structurally from the second affinity reagents, and in which the first affinity reagents and the second affinity reagents have similar, overlapping, or identical binding specificities (e.g., the first affinity reagents and the second affinity reagents may bind a same 3, 4, or 5 amino acid epitope but vary with respect to binding specificity for flanking sequences of the epitope). In this example, the differing sets of probes may be provided same or differing detectable labels.

It will be understood that, for a probe library comprising a plurality of multiplexed probe compositions, the same similar or differing characteristics set forth herein for probe libraries comprising a plurality of non-multiplexed probe compositions will also apply to the multiplexed probe compositions.

A probe library may comprise a first probe composition and a second probe composition, in which the first probe composition and the second probe composition are kinetically matched. In some embodiments set forth herein, the term “kinetically matched” can refer to the probes of two different probe compositions having similar or substantially identical binding kinetic properties (e.g., dissociation constant, on-rate constant, off-rate constant, etc.) for their respective binding targets. Kinetically matched probes may have binding kinetic properties that are within the same order of magnitude. Kinetically matched probes may have binding kinetic properties that differ by no more than about ±90%, ±80%, ±70%, ±60%, ±50%, ±40%, ±30%, ±25%, ±20%, ±15%, ±10%, ±5%, ±1%, or less than ±1%. For example, a first probe composition with probes having a dissociation constant of 5 nanomolar (nM) may be kinetically matched to a second probe composition with probes having a dissociation constant of 4 nM. In another example, a first probe composition with probes having a dissociation constant of 5 nanomolar (nM) may not be kinetically matched to a second probe composition with probes having a dissociation constant of 0.5 nM. For probes having more than one possible binding target (i.e., a primary binding target and a secondary binding target), two probe compositions may be kinetically matched with respect to their respective primary and/or secondary binding targets. A probe library may comprise a first probe composition and a second probe composition, in which the first probe composition and the second probe composition are not kinetically matched.

Two differing affinity reagents may have differing binding kinetics for their respective binding targets. The two differing affinity reagents may become kinetically matched when formulated into differing probe compositions by compositions set forth herein. For example, a first affinity reagent may be formulated into a first probe having a first probe architecture, as set forth herein, and a second affinity reagent may be formulated into a second probe having a second probe architecture, as set forth herein, in which the first probe architecture differs from the second probe architecture, in which the first affinity reagent is not kinetically matched to the second affinity reagent, and in which the first probe is kinetically matched to the second probe. In another example, a first probe may be provided in a first fluid composition, as set forth herein, and a second probe may be provided in a second fluid composition, as set forth herein, in which the first fluid composition differs from the second fluid composition, in which the first affinity reagent is not kinetically matched to the second affinity reagent, and in which the first probe is kinetically matched to the second probe in the presence of their respective fluid compositions.

Probe compositions of a probe library may be distinguished by the average valency of the probes comprising each respective probe composition. In accordance with some embodiments set forth herein, the term “valency” can refer to the total number of target-binding regions contained in a single probe. For example, an antibody may have 2 complementarity-determining regions. Accordingly, a probe formed by joining 5 antibodies may have a total valency of 10. Valency of a probe may be determined in part by the valency of a constitutive affinity reagent and/or a total quantity of constitutive affinity reagents. Probe valency can be increased by providing probes with greater quantities of affinity reagents, or by providing affinity reagents that are engineered to have a greater valency (see for example US Pat. App. Pub. No. 20200157190A1, which is herein incorporated by reference in its entirety). Compositions and methods of use of multivalent probes are described in U.S. Pat. No. 11,692,217, which is herein incorporated by reference in its entirety. Increased valency of a probe may produce an avidity effect that increased the amount of time a probe remains associated with a binding target. Accordingly, providing a probe composition with an increased probe valency may facilitate kinetic matching of the probe composition to a second probe composition containing lower-valency probes that inherently bind their target more strongly. An average valency of a probe composition can refer to the average of the individual valencies of each probe of a probe composition. For example, an equimolar mixture of probes having a valency of 2 and probes having a valency of 4 might have an average valency of 3. A probe composition may have an average valency of at least about 1, 2, 3, 4, 5, 6, 7, 8, 9, 10, 12, 14, 16, 18, 20, 22, 24, 26, 28, 30, 40, 50, 100, or more than 100. Alternatively or additionally, a probe composition may have an average valency of no more than about 100, 50, 40, 30, 28, 26, 24, 22, 20, 18, 16, 14, 12, 10, 9, 8, 7, 6, 5, 4, 3, 2 or less than 2. A first probe composition may have an average valency that is at least about 1.1×, 1.2×, 1.5×, 2×, 3×, 4×, 5×, 10×, 20×, 50×, 100×, or more than 100× the average valency of a second probe composition. Alternatively or additionally, a first probe composition may have an average valency that is no more than about 100×, 50×, 20×, 10×, 5×, 4×, 3×, 2×, 1.5×, 1.2×, 1.1×, or less than 1.1× the average valency of a second probe composition.

A probe of a probe composition may comprise a linking moiety, as set forth herein. A linking moiety may comprise any suitable material that can join two probe components. A linking moiety may attach a detectable label or tether strand to an affinity reagent. A linking moiety may couple two or more affinity reagents together. A linking moiety may attach two or more affinity reagents to additional probe components, such as a detectable label and/or a tether strand. A linking moiety may comprise a polymer chain. A polymeric linking moiety may be a linear polymer chain or a branched polymer chain (e.g., a dendron or dendrimer molecule). Branched polymers with reactive functional groups on some or all branches of the polymer may be particularly useful for joining together probe components. A linking moiety may comprise a nanoparticle, such as a nucleic acid nanoparticle. A probe of a first probe composition may be distinguished from a probe of a second probe composition by presence or absence of a linking moiety, or by differences in respective linking moieties. For example, a low valency probe of a first probe composition may be provided by attaching a single affinity reagent to a branched polymeric linking moiety containing a plurality of attached detectable labels. A high valency probe of a second composition may be provided by attaching a plurality of affinity reagents and a plurality of fluorescent labels to a nucleic acid nanoparticle. A probe may comprise only one linking moiety, in which each probe component is attached to the linking moiety. A probe may comprise two or more linking moieties. For example, a probe may be formed by attaching two or more polymeric linking moieties to an affinity reagent, in which each of the polymeric linking moieties comprises a plurality of fluorescent labels. Linking moieties may be attached to an affinity reagent in a site-specific manner (e.g., attachment of linking moieties or pluralities thereof to terminal amino acids or disulfide bridges of antibodies) or in a stochastic fashion (e.g., random attachment of linking moieties or pluralities thereof to reactive amino acid sidechains). A first probe composition may differ from a second probe composition with respect to the attachment type of linking moieties to the respective probes. For example, a first probe composition may comprise linking moieties stochastically attached to antibodies and a second probe composition may comprise antibodies with site-specific attachment of linking moieties. A probe of a probe composition may comprise at least about 1, 2, 3, 4, 5, 6, 7, 8, 9, 10, or more than 10 linking moieties. Alternatively or additionally, a probe of a probe composition may comprise no more than about 10, 9, 8, 7, 6, 5, 4, 3, 2, or less than 2 linking moieties. A probe of a probe composition may comprise no linking moieties. A probe of a first probe composition may comprise a greater quantity of linking moieties than a probe of a second probe composition.

A probe of a probe composition may comprise one or more detectable labels, as set forth herein. Detectable labels may be attached to an affinity reagent in a site-specific manner (e.g., attachment of detectable labels or pluralities thereof to terminal amino acids or disulfide bridges of antibodies) or in a stochastic fashion (e.g., random attachment of detectable labels or pluralities thereof to reactive amino acid sidechains). A first probe composition may differ from a second probe composition with respect to the attachment type of detectable labels to the respective probes. For example, a first probe composition may comprise stochastically labeled antibodies and a second probe composition may comprise antibodies with site-specific labeling. A first probe composition may differ from a second probe composition with respect to the average quantity of detectable labels attached probes of each respective probe composition. For example, probes of a first probe composition may have an average of 10 attached fluorescent labels while probes of a second probe composition may have an average of 20 attached fluorescent labels. A probe may comprise at least about 1, 2, 5, 10, 15, 20, 25, 30, 35, 40, 45, 50, 60, 70, 80, 90, 100, or more than 100 detectable labels. Alternatively or additionally, a probe may comprise no more than about 100, 90, 80, 70, 60, 50, 45, 40, 35, 30, 25, 20, 15, 10, 5, 2, or less than 2 detectable labels. A probe of a probe composition may comprise no detectable labels. A probe of a first probe composition may comprise a greater quantity of detectable labels than a probe of a second probe composition.

A probe of a probe composition may comprise one or more tether strands, as set forth herein. Tether strands may be attached to an affinity reagent in a site-specific manner (e.g., attachment of tether strands or pluralities thereof to terminal amino acids or disulfide bridges of antibodies) or in a stochastic fashion (e.g., random attachment of tether strands or pluralities thereof to reactive amino acid sidechains). A first probe composition may differ from a second probe composition with respect to the attachment type of tether strands to the respective probes. For example, a first probe composition may comprise stochastic attachment of tether strands to antibodies and a second probe composition may comprise antibodies with site-specific attachment of tether strands. A first probe composition may differ from a second probe composition with respect to the average quantity of tether strands attached to probes of each respective probe composition. For example, probes of a first probe composition may have an average of 2 attached tether strands while probes of a second probe composition may have an average of 10 attached tether strands. A probe may comprise at least about 1, 2, 3, 4, 5, 6, 7, 8, 9, 10, 15, 20, 25, 30, 35, 40, 45, 50, 60, 70, 80, 90, 100, or more than 100 tether strands. Alternatively or additionally, a probe may comprise no more than about 100, 90, 80, 70, 60, 50, 45, 40, 35, 30, 25, 20, 15, 10, 9, 8, 7, 6, 5, 4, 3, 2, or less than 2 tether strands. A probe of a probe composition may comprise no tether strands. A probe of a first probe composition may comprise a greater quantity of tether strands than a probe of a second probe composition.

A probe library may comprise a first probe composition and a second probe composition, in which the probes of the first probe composition and the probes of the second probe composition are substantially identical, and in which the fluid composition of the first probe composition differs from the fluid composition of the second probe composition. Accordingly, the first probe composition may not be kinetically matched to the second probe composition. Such a configuration of a probe library may be useful for distinguishing analytes based upon differential binding of a same probe in the presence of differing fluid compositions.

A probe library may comprise a first probe composition and a second probe composition, in which the first probe composition is kinetically matched to the second probe composition. A probe library may comprise a first probe composition and a second probe composition, in which the first probe composition is not kinetically matched to the second probe composition.

A probe library may comprise a first probe composition and a second probe composition, in which a probe of the first probe composition comprises a first affinity reagent, and in which a probe of the second probe composition comprises a second affinity reagent. In some configurations, the first affinity reagent of the first probe composition has an identical chemical structure (e.g., identical residue sequence, identical complementarity-determining region, etc.) to the second affinity reagent of the second probe composition. Probes of two differing probe compositions, each containing structurally identical affinity reagents, may have differing binding specificities if the probe architecture or fluid composition differs between the two differing probe compositions. Probes of two differing probe compositions, each containing structurally identical affinity reagents, may have identical binding specificities, but the probe compositions may differ with respect to probe concentration.

In other configurations, the first affinity reagent of the first probe composition has a different chemical structure from the second affinity reagent of the second probe composition. A first affinity reagent of a first probe composition may differ from a second affinity reagent of a second probe composition with respect to type of affinity reagent. For example, a first affinity reagent of a first probe composition may be selected from amongst a type of affinity reagent consisting of an antibody, Fab′ fragment, F(ab′)2 fragment, single-chain variable fragments, di-scFv, tri-scFv, or microantibody, nucleic acid aptamer, affibody, affilin, affimer, affitin, alphabody, anticalin, avimer, miniprotein, DARPin, monobody, nanoCLAMP, lectin, carbohydrate, and Major Histocompatibility Complex (MHC) Class II molecule, and a second affinity reagent of a second probe composition may be selected from amongst the same types of affinity reagents excluding the type of the first affinity reagent. Alternatively, the first affinity reagent of the first probe composition and the second affinity reagent of the second probe composition can be the same type of affinity reagent (e.g., both can be antibodies, both can be aptamers, etc.). In some configurations, a first affinity reagent can differ from the second affinity reagent with respect to chemical structure of a paratope or complementarity-determining region. For example, a first antibody affinity reagent of first probe composition may differ from a second antibody affinity reagent of a second probe composition with respect to amino acid sequence of their respective paratopes. In another example, a first aptamer affinity reagent may differ from a second aptamer affinity reagent of a second probe composition with respect to nucleotide sequence. In some configurations, a first affinity reagent of a first probe composition can differ from a second affinity reagent of a second probe composition with respect to a quantity of paratopes. A first affinity reagent of a first probe composition can differ from a second affinity reagent of a second probe composition with respect to a valency of paratopes. For example, a first affinity reagent of a first probe composition can be an antibody comprising 2 paratopes and a second affinity reagent of a second probe composition can be an antibody comprising more than 2 paratopes. Valency of affinity reagents may arise naturally (e.g., natural IgG antibodies having a valency of 2 and natural IgM antibodies having a valency of 10) or may be engineered (e.g., an IgG engineered to have a valency of 4).

A probe library may comprise a first probe composition and a second probe composition, in which a probe of the first probe composition comprises a first affinity reagent, in which a probe of the second probe composition comprises a second affinity reagent, and in which a probe of the first probe composition can have an identical probe architecture to a probe of the second probe composition. A probe library may comprise a first probe composition and a second probe composition, in which a probe of the first probe composition comprises a first affinity reagent, in which a probe of the second probe composition comprises a second affinity reagent, in which a probe of the first probe composition can have an identical probe architecture to a probe of the second probe composition, and in which a first fluidic composition of the first probe composition differs from a second fluidic composition of the second probe composition.

A probe library may comprise a first probe composition and a second probe composition, in which a probe of the first probe composition comprises a first affinity reagent, in which a probe of the second probe composition comprises a second affinity reagent, and in which a first fluidic medium of the first probe composition is substantially identical to a second fluidic medium of the second probe composition. A probe library may comprise a first probe composition and a second probe composition, in which a probe of the first probe composition comprises a first affinity reagent, in which a probe of the second probe composition comprises a second affinity reagent, in which a first fluidic medium of the first probe composition is substantially identical to a second fluidic medium of the second probe composition, and in which a first probe architecture of a probe of the first probe composition differs from a second probe architecture of a probe of the second probe composition.

A probe library may comprise a first probe composition and a second probe composition, in which the first probe composition has a different probe concentration than the second probe composition. A probe library may comprise a first probe composition and a second probe composition, in which the first probe composition has a different total quantity of probes than the second probe composition. A probe library may comprise a first probe composition and a second probe composition, in which the first probe composition has a different probe concentration and/or total quantity of probes than the second probe composition, and in which the first probe composition is substantially the same as the second probe composition with respect to one or more of chemical structure of an affinity reagent, probe architecture, and fluid composition. A probe library may comprise a first probe composition and a second probe composition, in which the first probe composition has a different probe concentration and/or total quantity of probes than the second probe composition, and in which the first probe composition differs from the second probe composition with respect to one or more of chemical structure of an affinity reagent, probe architecture, and fluid composition.

In some embodiments set forth herein, the term “fluid composition,” when used in reference to a probe composition, can refer to the chemical composition of a fluidic medium of a probe composition excluding the probes. A fluid composition can include at least one liquid solvent species, and optionally at least one component selected from the group consisting of: i) an ionic species, ii) a buffering species, iii) a surfactant species, iv) a blocking agent, v) an excipient agent, and vi) a detection agent. A detection agent can include any suitable labeled secondary probe or labeled reagent that is configured to bind to a probe of a probe composition, thereby providing a detectable characteristic to the probe. Examples of detection agents could include fluorescently-labeled secondary antibodies, a fluorescent or luminescent label attached to a component of a ligand-receptor pair (e.g., streptavidin/biotin; SpyCatcher/SpyTag, etc.), and a fluorescent or luminescent label attached to a reactive functional group (e.g., a Click-type reagent). Accordingly, a probe of a probe composition may be provided a moiety (e.g., a tag, a component of a ligand-receptor pair, a reactive functional group) that is complementary to or configured to form a binding interaction with a detection agent. Useful fluid compositions and constituents thereof for probe compositions are described in US Pat. App. Pub. No. 20240192202A1, which is incorporated by reference in its entirety.

The formulation of a fluid composition for a probe composition can vary according to the formulation of the probes comprising the probe composition. For example, a probe containing a certain affinity reagent may bind most optimally at a particular pH or ionic strength. The fluid composition formulated for this probe may differ from the formulation of a fluid composition provided to a probe containing a differing affinity reagent. In another example, a fluid composition may be formulated for a particular retaining moiety, linking moiety, tether, or detectable label of a probe to inhibit the formation of orthogonal or non-specific binding interactions. The skilled person will recognize that fluid compositions may be varied between probe compositions to provide the most optimal storage and/or binding conditions for each respective probe composition. Further, the skilled person will recognize that differing probe compositions may be provided a substantially same fluid composition if the fluid composition can produce useful binding interactions from each of the differing probe compositions. Methods of determining optimal fluid compositions for probe compositions are described in US Pat. App. Pub. No. 20240192202A1, which is incorporated by reference in its entirety.

Accordingly, a first fluidic composition of a first probe composition may differ from a second fluidic composition of a second probe composition with respect to one or more of: i) presence of a solvent species; ii) concentration or weight percentage of the solvent species; iii) presence of an ionic species; iv) concentration or weight percentage of the ionic species; v) presence of a buffering species; vi) concentration or weight percentage of the buffering species; vii) presence of a surfactant species; viii) concentration or weight percentage of the surfactant species; ix) presence of a blocking agent; x) concentration or weight percentage of the blocking agent; xi) presence of an excipient agent; xii) concentration or weight percentage of the excipient agent; xiii) presence of a detection agent; and xiv) concentration or weight percentage of the detection agent. In some configurations, the first fluidic composition of the first probe composition can differ from the second fluidic composition of the second probe composition with respect to two or more, three or more, four or more, five or more, six or more, seven or more, eight or more, nine or more, or ten or more of the aforementioned fluid composition properties.

In some embodiments set forth herein, the term “probe architecture,” when used in reference to a probe, can refer to the spatial arrangement of components contained in the probe. The spatial arrangement of components of a probe can vary according to the quantity of components joined in a probe and/or the attachments that join the components to each other in the probe. The probe architecture of a first probe having two joined affinity reagents each attached to a single retaining moiety can differ from the probe architecture of a second probe having three joined affinity reagents each attached to a single retaining moiety due to the difference in quantity of affinity reagents. In another example, the probe architecture of an antibody attached to a detectable label by a biotin-streptavidin linkage can differ from the probe architecture of an antibody attached to a detectable label by a linking moiety inserted into a disulfide bridge of the antibody (see for example US Pat. App. Pub. No. 20240053333A1, which is incorporated by reference in its entirety).

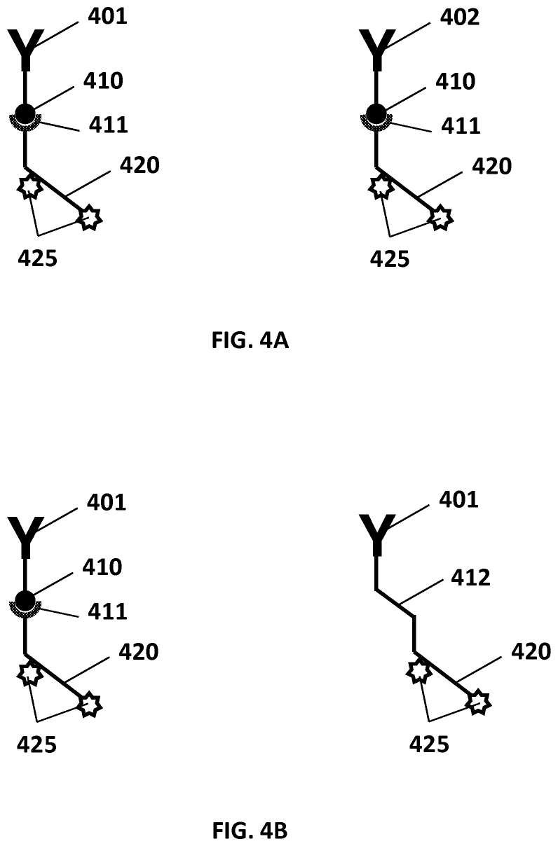

Certain affinity reagents such as antibodies may comprise a conserved or invariant portion of their structures (i.e., a structure that is common to different affinity reagents) and a variable portion (i.e., a structure containing an analyte-specific binding residue sequence). Two affinity reagents of differing binding specificity may have the same probe architecture if the affinity reagents have common conserved or invariant structures, and identical probe components (e.g., detectable labels, retaining moieties, linking moieties, tether strands, etc.) attached to the conserved or invariant structures by structurally identical linkages. FIGS. 4A-4B illustrate aspects of probe architecture. FIG. 4A depicts two antibody-based probes having differing antibodies and identical probe architecture. The left probe comprises a first antibody 401 and the right probe comprises a second antibody 402, in which the antibodies differ (e.g., with respect to binding specificity, with respect to valency, etc.). The first antibody 401 and the second antibody 402 are each individually attached to a linking moiety 420 that is attached to two fluorescent labels 425. The linking moiety 420 is attached to each respective affinity reagent by binding of a first coupling moiety 410 attached to the antibody to a second coupling moiety 411 attached to the linking moiety 420 (e.g., streptavidin-biotin, SpyCatcher-SpyTag, etc.). For each affinity reagent, the first coupling moiety 410 is coupled to the same attachment site constant region of the antibody. Although the antibodies differ between the left probe and right probe, both probes may be considered to have a same probe architecture due to the identical structures of the components attached to the antibodies and the use of identical attachment sites in the constant regions of the affinity reagent structures. FIG. 4B depicts two antibody-based probes having identical antibodies and differing probe architectures. The left probe containing the first antibody 401 has an identical probe architecture to the left probe of FIG. 4A. The right probe of FIG. 4B contains the first antibody 401 attached to the linking moiety 420 containing the two fluorescent labels 425 by a covalent bond 412. Although the antibodies 401 of the left probe and right probe are identical, the probe architectures differ due to the difference in attachment chemistry of the linking moiety 420 to the antibody 401 for the left and right probes.

A probe library may comprise a first probe composition and a second probe composition, in which the probe architecture of a probe of the first probe composition differs from a probe architecture of a probe of the second probe composition. The probe architecture of the probe of the first probe composition can differ from the probe architecture of the probe of the second probe composition with respect to one or more of: i) presence of a retaining component; ii) size of the retaining component; iii) composition of the retaining component; iv) attachment site of an affinity reagent, linking moiety, detectable label, or tether strand to the retaining component; v) attachment chemistry of the affinity reagent, linking moiety, detectable label, or tether strand to the retaining component; vi) presence of a linking moiety; vii) length of the linking moiety; viii) composition of the linking moiety; ix) attachment site of an affinity reagent, retaining component, detectable label, or tether strand to the linking moiety; x) attachment chemistry of the affinity reagent, retaining component, detectable label, or tether strand to the linking moiety; xi) presence of a species of detectable label; xii) quantity of the species of detectable label; xiii) attachment site of an affinity reagent, linking moiety, retaining component, or tether strand to the detectable label; xiv) attachment chemistry of the affinity reagent, linking moiety, retaining component, or tether strand to the detectable label; xv) presence of a tether strand; xvi) quantity of tether strands; xvii) composition of the tether strand; xviii) attachment site of an affinity reagent, linking moiety, retaining component, or detectable label to the tether strand; and xix) attachment chemistry of the affinity reagent, linking moiety, retaining component, or detectable label to the tether strand. In some configurations, the probe architecture of the probe of the first probe composition can differ from the probe architecture of the probe of the second probe composition with respect to two or more, three or more, four or more, five or more, six or more, seven or more, eight or more, nine or more, or ten or more of the aforementioned probe architecture properties. Table I provides exemplary differences in probe architecture between probes of a first probe composition and probes of a second probe composition.

TABLE I

| A probe of the first probe composition | A probe of the second probe composition |
|---|---|
| comprises: | can comprise: |
| A first type of affinity reagent | A different type of affinity reagent |
| | A same type of affinity reagent with a |
| | different target-binding region |
| | A greater quantity of the first type of affinity |
| | reagent |
| | A lesser quantity of the first type of affinity |
| | reagent |
| A linking moiety | No linking moieties |
| | A larger linking moiety |
| | A smaller linking moiety |
| | A different type of linking moiety |
| | A greater quantity of linking moieties |
| | A lesser quantity of linking moieties |
| A detectable label | No detectable labels |
| | A different type of detectable label |
| | A greater quantity of detectable labels |
| | A lesser quantity of detectable labels |
| A tether strand | No tether strands |
| | A different type of tether strand |
| | A longer tether strand |
| | A shorter tether strand |
| | A greater quantity of tether strands |
| | A lesser quantity of tether strands |
| A site-specific attachment site of a probe | A stochastic attachment site of the same |
| component (e.g., attachment to C- or N- | probe component (e.g., attachment to an |
| terminus or disulfide bridge of an antibody) | amino acid sidechain) |
| | A stochastic attachment site of a different |
| | probe component |
| | Attachment of the same probe component to |
| | a different site-specific attachment site |
| | Attachment of a different probe component |
| | to a different site-specific attachment site |
| Only one affinity reagent | Two or more affinity reagents |
| A single-affinity reagent | A multi-affinity reagent |

In some cases, modification of an affinity reagent (e.g., attachment of a detectable label, linking moiety, and/or tether strand) may alter the binding properties of the resultant probe. Alteration of affinity reagents may affect their binding kinetic properties and/or their mass transfer properties. Accordingly, certain affinity reagents of a probe library may be provided in an unmodified form (e.g., substantially devoid of any attached moieties, such as detectable labels, linking moieties, and/or tether strands). A probe library may comprise a first probe composition comprising a plurality of unmodified affinity reagents, and may further comprise a second probe composition comprising a plurality of modified affinity reagents (e.g., affinity reagents attached to detectable labels, linking moieties, and/or tether strands). The second probe composition may be substantially devoid of unmodified affinity reagents.

In some cases, an unmodified affinity reagent may be detectable by the inclusion of a secondary affinity reagent. A secondary affinity reagent may bind to a portion of an unmodified affinity reagent. For example, secondary antibodies are commercially available for common types of antibodies, such as IgG. In another example, antibody-binding proteins such as protein A or protein G may be useful as secondary affinity reagents. After binding an unmodified affinity reagent to a binding target, a labeled secondary affinity reagent may be bound to the unmodified affinity reagent, thereby providing a detectable signal at an address containing the bound unmodified affinity reagent. Accordingly, a probe library may comprise one or more probe compositions comprising secondary affinity reagents. In some cases, a probe library may comprise a first probe composition comprising a plurality of unlabeled probes and a second probe composition comprising a plurality of labeled secondary affinity reagents.

Secondary affinity reagents can include any affinity reagent that has a binding specificity for another affinity reagent or a portion thereof. For example, secondary antibodies are commercially available for primary antibodies, in which the secondary antibodies have a binding specificity for a non-binding portion of the primary antibodies (e.g., Fc regions). Useful secondary affinity reagents can include antibodies or functional fragments thereof (e.g., Fab′ fragments, F(ab′)2 fragments, single-chain variable fragments (scFv), di-scFv, tri-scFv, or microantibodies), affibodies, affilins, affimers, affitins, alphabodies, anticalins, avimers, DARPins, monobodies, nanoCLAMPs, nucleic acid aptamers, protein aptamers, lectins or functional fragments thereof.

Secondary affinity reagents may be particularly useful for probe compositions comprising antibodies. Certain antibodies or fragments thereof may be bound by secondary antibodies, secondary antibody fragments, or other antibody-binding molecules (e.g., Protein A, Protein G, Protein A/G, Protein L, Protein M, etc.).

A vessel, as set forth herein, may comprise a probe composition containing a primary affinity reagent and a secondary affinity reagent. The primary affinity reagent and the secondary affinity reagent may be provided in a particular stoichiometry that favors association of secondary affinity reagents to primary affinity reagents. A probe composition may have a molar ratio of primary affinity reagent to secondary affinity reagent of at least about 1:0.1, 1:0.5, 1:1, 1:1.5, 1:2, 1:3, 1:4, 1:5, 1:10, 1:20, 1:50, 1:100 or more than 1:100. Alternatively or additionally, a probe composition may have a molar ratio of primary affinity reagent to secondary affinity reagent of no more than about 1:100, 1:50, 1:20, 1:10, 1:5, 1:4, 1:3, 1:2, 1:1.5, 1:1, 1:0.5, 1:0.1, or less than 1:0.1. A probe library may comprise one or more vessels that contain secondary affinity reagents, in which the one or more vessels are substantially devoid of primary affinity reagents. Accordingly, a system may be configured to deliver a probe composition comprising an unlabeled primary affinity reagent to a plurality of analytes, and separately deliver a probe composition comprising a secondary affinity reagent to the plurality of analytes. In some configurations, a probe composition comprising an unlabeled primary affinity reagent and a probe composition comprising a secondary affinity reagent may be combined (e.g., with or without mixing) before being delivered to a plurality of analytes. In other configurations, a probe composition comprising an unlabeled primary affinity reagent may be delivered to a plurality of analytes before a probe composition comprising a secondary affinity reagent is delivered to the plurality of analytes.

A probe library may comprise two or more different probe compositions, in which each of the two or more different probe compositions comprise a primary affinity reagent and a secondary affinity reagent. In some configurations, a first probe composition and a second probe composition of a probe library may differ with respect to type of secondary affinity reagent (e.g., antibody vs. antibody fragment), concentration of secondary affinity reagent, type of detectable label attached to a secondary affinity reagent, or a combination thereof.

Two differing probe compositions may have a common probe architecture, probe concentration, and/or fluid composition while differing with respect to binding specificity.

Alternatively, two probe compositions may differ with respect to probe architecture, probe concentration, and/or fluid composition due to their respective binding specificities. Accordingly, a probe library may comprise a first probe composition and a second probe composition, in which the first probe composition differs from the second probe composition with respect to its binding specificity. Probes of a probe composition may be characterized as binding to only one binding target. Alternatively, probes of a probe composition may be characterized as binding to a plurality of binding targets.

A probe may be characterized as binding to a binding target if it meets certain criteria for detectability in the presence of the binding target. Detectability in an analyte detection assay may be based upon a modality of detection for the assay. For example, the necessary binding equilibrium or kinetic characteristics for a detectable probe may differ when the probe binding is observed via confocal scanning fluorescence microscopy versus total internal reflectance fluorescence microscopy. In some cases, a probe may be considered to have a binding specificity for a binding target if it has a characterized dissociation constant for the binding target of no more than about 1 micromolar (μM), 500 nM, 100 nM, 50 nM, 10 nM, 5 nM, 2 nM, 1 nM, 0.5 nM, 0.1 nM, 0.05 nM, 0.01 nM, or less than 0.01 nM. Accordingly, a probe may have a binding specificity for a plurality of binding targets if it meets the equilibrium or kinetic characteristic for each of the plurality of binding targets.

A probe library may comprise a first probe composition and a second probe composition, in which the first probe composition has a binding specificity for only one binding target, and in which the second probe composition has a binding specificity for two or more binding targets. A probe library may comprise a first probe composition and a second probe composition, in which the first probe composition has a binding specificity for a first set of two or more binding targets, and in which the second probe composition has a binding specificity for a second set of two or more binding targets, in which the first set of two or more binding targets differs from the second set of two or more binding targets.

The screening, selection, and characterization of affinity reagents or probes, including probes that bind to two or more binding targets is described in U.S. Pat. Nos. 11,970,693 and 11,993,865, and US Pat. App. Pub. No. 20250306015 and U.S. patent application Ser. No. 19/338,819, each of which is incorporated by reference in its entirety. Table II provides examples of binding specificities that may be useful for certain assays set forth herein. Probes in Table II may be classified as binding to either a single binding target (i.e., a single-affinity probe) or multiple binding targets (a promiscuous or multi-affinity probe). A probe library may comprise a first probe composition with a first type of binding specificity chosen from Table II, and a second probe composition with a second type of binding specificity chosen from Table II. A probe library may also contain a probe composition that has no binding specificity for analytes present in an assay. Such a probe composition may be useful as a negative control during certain assays. A probe library may also contain a probe composition that has a binding specificity to an engineered, synthetic, or exogenous protein that is provided as a control or standard analyte. Such a probe composition may be useful as a positive control during certain assays.

TABLE II

| Binding Type | Binding Specificity | Example |
|---|---|---|
| Single | Binds to a single specific analyte | EGFR-specific antibody |
| | | VEGF-specific aptamer |
| Single | Binds to a specific proteoform of a | aptamer specific to a single splice |
| | single protein | isoform of a protein |
| | | antibody specific to 2N4R Tau |
| | | proteoform |
| Multiple | Binds to an epitope X that is present | antibody that binds to trimer |
| | in many proteins or peptides (X | sequence DTR |
| | being a constant contiguous or non- | B1 aptamer that binds to any his- |
| | contiguous sequence of 2, 3, 4, 5, 6, | tagged protein |
| | or 7 amino acids) | |
| Multiple | Binds to a set of proteins containing | An antibody that binds to all 400 |
| | amino acid sequence αXβ, wherein | possible variations of αDTRβ, |
| | X is a constant sequence of 2, 3, 4, | where α and β are independently |
| | 5, 6, or 7 amino acids, and α and β | any of the 20 naturally-occurring |
| | are independently varying 1 or 2 | amino acids |
| | amino acid sequences | An antibody that binds to at least |
| | | 10% of the 400 possible variations |
| | | of αHSPβ, where α and β are |
| | | independently any of the 20 |
| | | naturally-occurring amino acids |
| Multiple | Binds to an epitope X that contains a | An antibody that binds to epitope |
| | post-translation modification (X | AYA only when the tyrosine is |
| | being a constant sequence of 2, 3, 4, | phosphorylated |
| | 5, 6, or 7 amino acids) | an antibody that binds to epitope |
| | | HCP only when the cysteine is |
| | | glycosylated |
| Multiple | Binds to any protein containing a | ubiquitin-specific affinity reagent |
| | specific post-translational | SUMO-specific affinity reagent |
| | modification | glycosylation-specific aptamer |
| Multiple | Binds to any protein containing a | aptamer specific to a cyclical |
| | modified terminal amino acid | phenylthiocarbamoyl Edman |
| | | complex derivative of glycine |
| | | antibody specific to a cyclical |
| | | phenylthiocarbamoyl Edman |
| | | complex derivative of leucine |
| Multiple | Binds to a tag sequence attached to | antibody that binds any avi-tagged |
| | or co-located with a plurality of | protein |
| | proteins | antibody that binds to any GST- |
| | | tagged protein |

Multi-affinity probes may be useful for certain methods of analyte characterization set forth herein. Some of those methods may further incorporate single-affinity probes. Single-affinity probes can be useful for identifying high-abundance proteins. For example, a human blood sample may contain high quantities of proteins such as albumins and globulins. Rapid identification of these proteins amongst a plurality of proteins can allow them to be excluded from analysis, thereby reducing the computational cost of analyzing a sample, or devoting more computational bandwidth to lower-abundance but more biologically significant proteins. Single-affinity probes can also serve as a useful control reagent. Certain proteins of known and varying abundances may serve as endogenous standards in a protein sample; single-affinity probes may facilitate quantification of these endogenous standard proteins, thereby facilitating quality control of sample characterization.

Single-affinity probes may also be useful for additional methods. Single-affinity probes may facilitate characterization of a specific protein proteoform and/or isoform. For example, a protein with N different independent post-translational modifications (PTMs) can have up to 2N proteoforms (i.e., each PTM can occur independently of the presence of absence of another PTM). Accordingly, the 2^{N} proteoforms may be identified by a probe library containing N single-affinity probes, each of the N differing probes having a binding specificity for a differing post-translational modification. In another example, a protein with M isoforms may be identified by a probe library containing no more than M single-affinity probes. In some cases, a protein with M isoforms may be identified by a probe library containing (M−1) single-affinity probes; any of the proteins not bound by any of the (M−1) probes can be identified as the isoform without a probe in the probe library. Accordingly a probe library may be used to characterize a particular protein, in which the set of probes utilized to characterize the particular protein may contain no more than about M+N unique probes (e.g., M+N−1 unique probes) amongst its probe compositions. Single-affinity probes can also be useful for methods that utilize Edman-type degradation, such as peptide sequencing methods. Such single-affinity probes may be specific to a single modified terminal amino acid.

A probe library may comprise at least one probe composition comprising single-affinity probes and at least one probe composition comprising multi-affinity probes. In some configurations, a probe library may comprise a greater quantity of probe compositions containing multi-affinity probes than the quantity of probe compositions containing single-affinity probes. Alternatively, a probe library may comprise a greater quantity of probe compositions containing single-affinity probes than the quantity of probe compositions containing multi-affinity probes. A probe library may comprise at least about 20 probe compositions (e.g., at least about 30, 40, 50, 75, 100, 150, 200, 300, 400, 500, or more than 500 probe compositions), in which at least about 5% (e.g., at least about 10%, 15%, 20%, 25%, 30%, 40%, 50%, 75%, 80%, 85%, 90%, 95%, or 100%) of the individual probe compositions contain single-affinity probes or multi-affinity probes. Alternatively or additionally, a probe library may comprise at least about 20 probe compositions, in which no more than about 95% (e.g., no more than about 90%, 85%, 80%, 75%, 50%, 40%, 30%, 25%, 20%, 15%, 10%, 5%, or less than 5%) of the individual probe compositions comprise single-affinity probes or multi-affinity probes. A probe library may comprise one or more probe compositions that are substantially devoid of single-affinity probes or multi-affinity probes. A probe library may comprise one or more probe compositions comprising a mixture of single-affinity probes and multi-affinity probes. A probe library may comprise a plurality of vessels, in which at least about 5% (e.g., at least about 10%, 15%, 20%, 25%, 30%, 40%, 50%, 75%, 80%, 85%, 90%, 95%, or 100%) of the vessels contain single-affinity probe compositions or multi-affinity probe compositions. Alternatively or additionally, a probe library may comprise a plurality of vessels, in which no more than about 95% (e.g., no more than about 90%, 85%, 80%, 75%, 50%, 40%, 30%, 25%, 20%, 15%, 10%, 5%, or less than 5%) of the vessels comprise single-affinity probe compositions or multi-affinity probe compositions.

A probe library may comprise one or more probe compositions containing probes that are configured to be detected at an equilibrium binding condition. Accordingly, a probe library may be provided to a system, as set forth herein, that is configured to detect a binding interaction between a probe and an analyte at the equilibrium binding condition. The system may be configured to implement a method, as set forth herein, in which the method includes detection of a binding interaction between a probe of a probe composition and an analyte at the equilibrium binding condition. Alternatively, a probe library may comprise one or more probe compositions containing probes that are configured to be detected at a non-equilibrium binding condition. Accordingly, a probe library may be provided to a system, as set forth herein, that is configured to detect a binding interaction between a probe and an analyte at the non-equilibrium binding condition. The system may be configured to implement a method, as set forth herein, in which the method includes detection of a binding interaction between a probe of a probe composition and an analyte at the non-equilibrium binding condition. Methods for detecting binding at a non-equilibrium condition may include one or more rinsing or wash steps that remove unbound probes from contact with analytes. Particularly advantageous systems and methods will facilitate single-analyte resolution of the binding interactions between probes and analytes, whether at equilibrium or non-equilibrium.

Detection of binding interactions between probes of a probe composition and analytes may be repeated, whether for an equilibrium-based detection system or method, or for a non-equilibrium detection system or method. For example, multiple detection steps or cycles during binding equilibrium between probes of a probe composition and a plurality of analytes may facilitate observation of the temporal binding dynamics between the probes and analytes, and could facilitate quantitation of the binding kinetics (e.g., association or dissociation constant, on-rate kinetic parameter, off-rate kinetic parameter, etc.) between the probes and analytes. In another example, multiple detection steps or cycles during non-equilibrium binding between probes of a probe composition and a plurality of analytes may facilitate observation of the dissociation of probes from analytes, and could facilitate quantitation of the off-rate kinetic parameter for the probes and analytes. Accordingly, a probe composition may be characterized by a quantity of detection steps or detection cycles in which probes of the probe composition are detected during a method, as set forth herein. After contacting a probe composition, as set forth herein, to a plurality of analytes, binding interactions of probes of the probe composition may be detected at least 1, 2, 3, 4, 5, 6, 7, 8, 9, 10, 15, 20, 25, 30, 40, 50, 100, 500, 1000, or more than 1000 times before bound probes are dissociated from analytes. Alternatively or additionally, after contacting a probe composition, as set forth herein, to a plurality of analytes, binding interactions of probes of the probe composition may be detected no more than 1000, 500, 100, 50, 40, 30, 25, 20, 15, 10, 9, 8, 7, 6, 5, 4, 3, 2, or less than 2 times.

Accordingly, a probe library may comprise a first probe composition and a second probe composition, in which the first probe composition and the second probe composition are each configured to be detected for a same quantity of detection steps or cycles by a system, as set forth herein, or during a method, as set forth herein. A probe library may comprise a set of probe compositions, in which each probe composition of the set of probe compositions are configured to be detected for a same quantity of detection steps or cycles by a system, as set forth herein, or during a method, as set forth herein. A probe library may comprise a first probe composition and a second probe composition, in which the first probe composition and the second probe composition are each configured to be detected for a differing quantity of detection steps or cycles by a system, as set forth herein, or during a method, as set forth herein. A probe library may comprise a first set of probe compositions and a second set of probe compositions, in which each probe composition of the first set of probe compositions are configured to be detected for a same first quantity of detection steps or cycles by a system, as set forth herein, or during a method, as set forth herein, in which each probe composition of the second set of probe compositions are configured to be detected for a same second quantity of detection steps or cycles by the system or during the method, and in which the first quantity of detection steps or cycles differs from the second quantity of detection steps or cycles.

### Systems and Methods for Utilizing Probe Libraries

Further provided herein are systems and methods that utilize probe libraries as set forth herein. A probe library, as set forth herein, may be provided to a system as a kit. A system may be configured to deliver probe compositions of a probe library, as set forth herein, to a fluidic device comprising a plurality of analytes. A system may be configured to perform a method of characterizing one or more analytes utilizing one or more probe compositions of a probe library, as set forth herein.

In an aspect, provided herein is a system, comprising: (a) a vessel, wherein the vessel comprises a plurality of analytes, (b) a probe library comprising a first probe composition and a second probe composition, and (c) a fluidic system, wherein the fluidic system is configured to deliver the first probe composition and the second probe composition to the vessel of the fluidic device, and wherein the fluidic system is further configured to perform the steps of: (i) incubating the first probe composition with the plurality of analytes in the presence of a first binding condition, (ii) removing the first probe composition from the vessel of the fluidic cartridge, and (iii) after removing the first probe composition from the vessel, incubating the second probe composition with the plurality of analytes in the presence of a second binding condition, wherein the first binding condition differs from the second binding condition.

In another aspect, provided herein is a method, comprising: a) binding probes of a first probe composition to a first set of analytes of a plurality of analytes in the presence of a first binding condition, b) detecting the probes of the first probe composition bound to the first set of analytes, c) after binding the probes of the first probe composition to the first set of analytes, binding probes of a second probe composition to a second set of analytes of the plurality of analytes in the presence of a second binding condition, and d) detecting the probes of the second probe composition bound to the second set of analytes. In some cases, detecting probes of a first probe composition bound to a first set of analytes or detecting probes of a second probe composition bound to a second set of analytes can occur at single-analyte resolution. In other cases, detecting probes of a first probe composition bound to a first set of analytes or detecting probes of a second probe composition bound to a second set of analytes can occur at bulk resolution. In some cases, detecting probes of a first probe composition bound to a first set of analytes and detecting probes of a second probe composition bound to a second set of analytes can occur at single-analyte resolution. In other cases, detecting probes of a first probe composition bound to a first set of analytes and detecting probes of a second probe composition bound to a second set of analytes can occur at bulk resolution.

A system may comprise a vessel, in which the vessel comprises a plurality of analytes. A method implemented on a system may comprise a step of providing the plurality of analytes to the vessel. In some configurations, a vessel that is configured to contain a plurality of analytes may comprise a well (e.g., a well of a multi-well plate). In other configurations, a vessel that is configured to contain a plurality of analytes may comprise a fluidic cartridge or flow cell. A fluidic cartridge or flow cell may comprise one or more void spaces or volumes that are configured to receive analytes. In some cases, an array of sites may be disposed on a surface of a fluidic cartridge or flow cell. An array of sites on a surface of a fluidic cartridge or flow cell may be configured to immobilize analytes of a plurality of analytes on the surface. A single-analyte array is a useful configuration for some single-analyte methods set forth herein. For a single-analyte array, each site of an array of sites may be attached to only one analyte of the plurality of analytes. In some configurations of a single-analyte array, each site of an array of sites may be optically resolvable from each other site of the array of sites.

A vessel may comprise a plurality of analytes, in which analytes of the plurality of analytes are attached to a bead or particle. Attachment of analytes to a bead or particle may facilitate certain fluid transfer processes (e.g., removal of fluids, rinsing processes, etc.) by inhibiting loss of analytes from a vessel containing the bead or particle. A bead or particle may be configured to sediment on a surface of a vessel containing the bead or particle. Sedimentation of a bead or particle may occur passively (e.g., gravitational settling) or actively (e.g., by magnetophoresis, electrophoresis, or centrifugation). Accordingly, a system may further comprise a sedimentation device, wherein the sedimentation device is configured to couple the bead or particle to a surface of a vessel. A system may be configured to sediment a bead or particle to a surface of a vessel at any conceivable time during a method, such as after a step of incubating analytes with a probe composition, or before removing a fluidic medium from the vessel. A sedimentation device may be configured to produce a magnetic field, an electric field, or a rotational motion in the well. Accordingly, useful bead or particle compositions can include magnetic beads or particles, electrically-charged beads or particle, or high-density beads or particles (e.g., ceramic, semiconductor, mineral, metal, or metal oxide beads or particles).

A system may be configured to receive a probe library, as set forth herein. A probe library may be provided as a kit that is housed in a system. A system may comprise a housing, holder, or mating component that retains a probe library in the system. For example, a system may comprise a holder or mounting device that is configured to hold a probe library provided in a plate or cartridge format. In some configurations, a probe library may be provided to a system in a feedable format, such as a roll or sheet, in which the feedable format comprises a plurality of vessels comprising probe compositions. Accordingly, a system may comprise a mount for the feedable format and a device that is configured to feed the feedable format through the device.

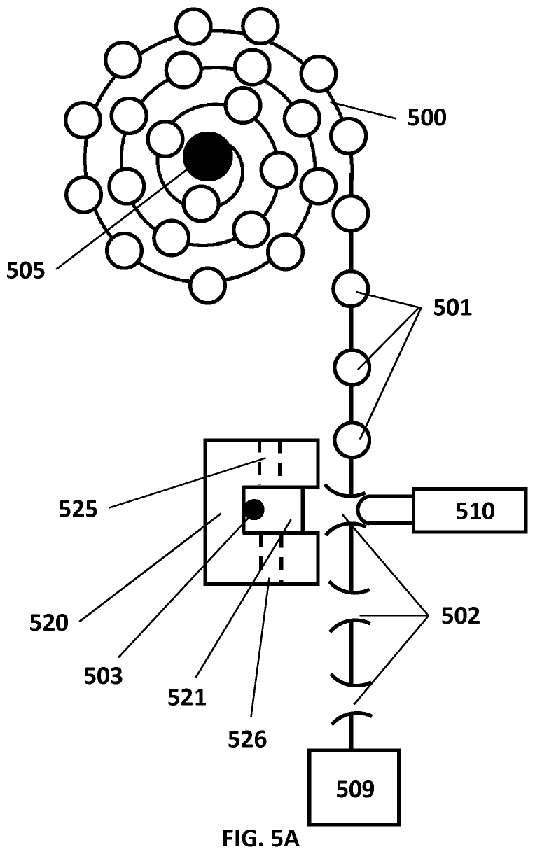

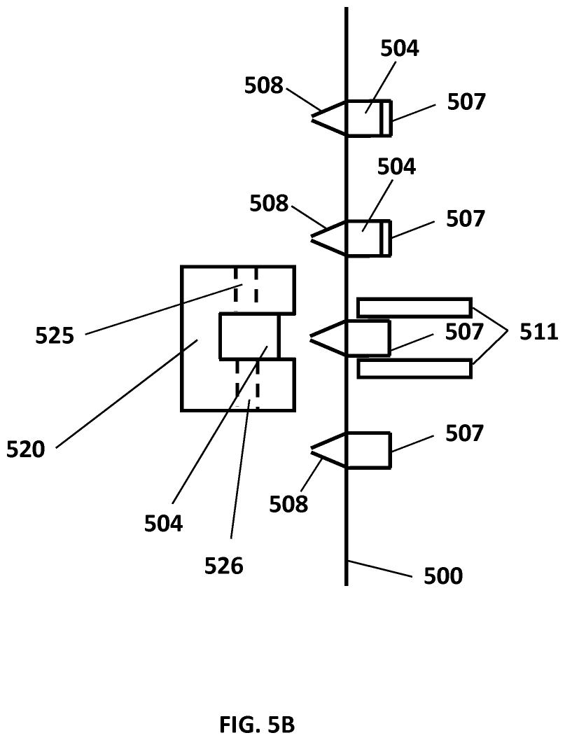

FIG. 5A depicts a system comprising a feedable format of a probe library. The probe library is provided in a spool 500 comprising a plurality of sealed compartments 501. Each sealed compartment comprises a probe composition in a solid phase composition 503 (e.g., a pellet). The spool 500 is mounted into the system on a spindle 505 or similar mount. The spool 500 is fed through the system by a feeding device 509 that unfurls the spool 500 into a sheet or chain. The feeding device 509 translates sealed compartments 501 past a vessel 520. As a sealed compartment 501 is in proximity to the vessel, a rupturing device 510 can rupture the sealed compartment 501, thereby forming an opened compartment 502. The rupturing device can operate by, for example, mechanically tearing a seal on a sealed compartment 501, or by providing a pressure that ruptures the seal on the sealed compartment 501. Once the compartment is opened, the solid phase composition 503 of a probe composition can be transferred into the vessel 520. The vessel may be filled with a fluidic medium 521 that is configured to dissolve the solid phase composition 503. Optionally, the vessel 520 may comprise one or more ports (525, 526) that facilitate fluid transfer into or out of the vessel 520. Alternatively, fluid can be delivered or removed from a same port through which the solid phase composition 503 is delivered into the vessel 520.

FIG. 5B depicts an alternative configuration of a system comprising a feedable format of a probe library. The feedable format comprises a spool 500 containing a plurality of compartments 507, optionally comprising a solid phase composition 503 or a liquid phase composition 504. In some configurations, the compartments 507 may be sealed (as in FIG. 5A), while in other configurations, a compartment 507 may comprise a port 508 (e.g., a septum, nozzle, or nipple) that facilitates fluid transfer out of the compartment 507. Transfer of a probe composition from a compartment 507 of the spool 500 may occur by a device 511 such as a pincer or piston that applies a pressure to the liquid phase composition 504, thereby displacing it from the compartment 507 into the vessel 520.

A system, such as a system like those depicted in FIGS. 5A and 5B, may comprise a plurality of vessels, each vessel individually configured to receive a single probe composition from a probe library (e.g., a probe library in a feedable format). For example, individual probe compositions of a feedable format may be distributed into wells of a multi-well plate.

Alternatively, a system may comprise a single vessel that is configured to receive a plurality of probe compositions sequentially. For example, the system of FIG. 5A may be configured to transfer a single solid phase composition 503 into the vessel 520, dissolve the solid phase composition 503 in a fluidic medium 521, then discharge the fluidic medium 521 comprising the dissolved solid phase composition 503 before transferring the next solid phase composition 503 into the vessel 520.

A system may comprise a fluidic system that is configured to deliver a probe composition from a probe library to a vessel of the fluidic device (e.g., a vessel containing a plurality of analytes). The fluidic system may comprise tubing, piping, valves, flow controllers, manifolds, and any other conceivable component that provides fluidic communication and control thereof between a fluidic device and a probe library. In some configurations, a fluidic system may comprise an autopipette or an automated liquid handler. An autopipette or an automated liquid handler may be configured to obtain a volume of fluid from a first vessel and transfer the volume of fluid to a second vessel, in which the first vessel and the second vessel are not in fluidic communication (i.e., not connected by a fluidic channel). A fluidic system may comprise one or more reservoirs, in which each reservoir is configured to contain a fluidic medium. A reservoir may store a fluidic medium for various purposes depending upon a method to be implemented on the system. Fluidic media stored in reservoirs of a system can include a fluidic medium for dissolving a solid phase probe composition into a fluid phase, a probe association medium, a probe detection medium, a rinsing medium, a probe dissociation medium, an acidifying agent, an alkalizing agent, a diluent, and a brine solution.

A fluidics system may further comprise a mixing vessel. A fluidic system may be configured to deliver a probe composition (e.g., a solid phase probe composition, a liquid phase probe composition) and a fluidic medium, as set forth herein, to a mixing vessel, thereby mixing the probe composition with the fluidic medium. A mixing vessel may comprise a mixing device, such as an impeller, to facilitate mixing of a probe composition with a fluidic medium. A fluidic system may be configured to alter a property (e.g., pH, ionic strength, composition, etc.) of a fluidic medium comprising a probe composition before delivering the fluidic medium to a vessel comprising a plurality of analytes.

A system may comprise a temperature-altering device. A temperature-altering device may be configured to increase or decrease the temperature of a fluidic medium (e.g., a fluidic medium comprising a probe composition, a fluidic medium that is substantially devoid of a probe composition). In some cases, a temperature-altering device may be incorporated into the fluidics system. For example, a heat exchanger or heater may be coupled to a section of tubing or a vessel comprising a fluidic medium. A system may be configured to alter a temperature of a fluidic medium before delivering the fluidic medium to a vessel (e.g., a mixing vessel, a vessel comprising a plurality of analytes) or a fluidic device (e.g., a fluidic cartridge or flow cell). In some cases, a temperature-altering device may be coupled to a fluidic device (e.g., a fluidic cartridge or flow cell). For example, a thermoelectric heat exchange device (e.g., a Pelletier device) may be coupled to a fluidic cartridge or flow cell. A system may be configured to alter a temperature of a fluidic medium after delivering the fluidic medium to a vessel (e.g., a mixing vessel, a vessel comprising a plurality of analytes) or a fluidic device (e.g., a fluidic cartridge or flow cell).

A probe library, as provided to a system set forth herein, may comprise a single vessel that is utilized to provide two or more probe compositions. A system may be configured to provide two or more probe compositions from a single vessel by: i) removing two volumes of fluidic media from the single vessel; and ii) altering a fluidic property (e.g., temperature, probe concentration, ionic strength, pH, chemical composition, etc.) of at least one of the two volumes of fluidic media, thereby providing two differing probe compositions. In some configurations, a fluidic system can be configured to: i) transfer a first volume of a fluid from a vessel, thereby providing a first probe composition; ii) transfer a second volume of the fluid from the vessel; and iii) mix the second volume of fluid with a first volume of a fluidic medium (e.g., a diluent, an acidifying agent, an alkalizing agent, etc.), thereby providing a second probe composition. In some configurations, a system may be further configured to mix the first volume of fluid with a second volume of the diluent, in which the first volume of the diluent differs from the second volume of the diluent (i.e., a system configured to provide two different dilutions of the same probe composition, thereby forming two differing probe compositions).

A system that utilizes a probe library may be configured to provide a first probe composition and a second probe composition, in which the first probe composition is provided with a first binding condition, and the second probe composition is provided with a second binding condition, in which the first binding condition differs from the second binding condition. In some embodiments set forth herein, the term “binding condition” can refer to the physical environment that contacts a binding partner and a probe of a probe composition during the association, binding, and/or dissociation of the binding partner to the probe during a binding interaction. When separately contacted to the same binding partner, two different probe compositions may have substantially identical binding conditions if both probe compositions have substantially identical fluid compositions. A binding condition may be characterized by one or more fluidic properties, including chemical composition and respective concentrations or molarities, pH, ionic strength, viscosity, density, capillarity, surface tension, heat capacity, thermal conductivity, temperature, fluid pressure, fluid velocity, electrical charge density, and magnetic permeability. Two probe compositions having differing fluid compositions may have substantially identical binding conditions if one or more aforementioned fluidic properties are substantially identical between the differing fluid compositions when contacted to a binding partner. Conversely, two probe compositions having the same fluid composition may have different binding conditions if one or more aforementioned fluidic properties differ between the fluid compositions when contacted to a binding partner. For example, two probe compositions having the same fluid composition may have different binding conditions if the temperature, fluid pressure, or fluid velocity differs between the two probe compositions when contacted to a binding partner.

A system that utilizes a probe library may be configured to provide a first probe composition and a second probe composition, in which the first probe composition is provided with a first binding condition, and the second probe composition is provided with a second binding condition, in which the first binding condition differs from the second binding condition. In some cases, a first binding condition of a first probe composition may inherently differ from a second binding condition of a second probe composition because the fluid composition of the first probe composition differs from the fluid composition of the second fluid composition. Accordingly, providing the first binding condition and providing the second binding condition may comprise delivering the first probe composition to a plurality of analytes (e.g., in a vessel), and delivering the second probe composition to the plurality of analytes.

In other cases, a system may be configured to alter a probe composition, thereby providing a binding condition when the probe composition is contacted to an analyte or a plurality thereof. A system may be configured to provide a first binding condition and a second binding condition, in which the first binding condition differs from the second binding condition with respect to temperature. Accordingly, a system may be configured to alter a temperature of a probe composition. A system may be configured to provide a first probe composition at a first fluid temperature, and the system may be further configured to provide a second probe composition at a second fluid temperature, wherein the first fluid temperature differs from the second fluid temperature. A system may alter a fluid temperature before delivering a probe composition to an analyte or a plurality thereof. Accordingly, in some configurations, a fluidic system may comprise a temperature-altering device that alters or controls the temperature of a fluidic medium comprising a probe composition. A system may alter a fluid temperature after delivering a probe composition to an analyte or a plurality thereof. Accordingly, in some configurations, a temperature-altering device may be coupled to a vessel comprising the analyte or the plurality thereof. In some cases, altering the temperature of a fluid comprising a probe composition can comprise mixing the fluid comprising the probe composition with a second fluid, in which the fluid comprising the probe composition has a different temperature than the second fluid. Mixing of a fluid comprising a probe composition with a second fluid to alter the temperature of the probe composition can occur before delivering the probe composition to an analyte or a plurality thereof. Mixing of a fluid comprising a probe composition with a second fluid can occur in a vessel comprising an analyte or a plurality thereof.

A system may be configured to provide a first binding condition and a second binding condition, in which the first binding condition differs from the second binding conditions with respect to pH, ionic strength, or probe concentration. A system may alter the binding condition of a probe composition by mixing a probe composition with a fluidic medium (e.g., a diluent, an acidifying agent, an alkalizing agent, etc.). Mixing of a fluid comprising a probe composition with a fluidic medium can occur before delivering the probe composition to an analyte or a plurality thereof. Mixing of a fluid comprising a probe composition with a fluidic medium can occur in a vessel comprising an analyte or a plurality thereof.

In some cases, a system may provide a first probe composition having a first binding condition, and may further provide a second probe composition having a second binding condition, in which a probe of the first probe composition is structurally identical to a probe of the second probe composition, and in which the first binding condition differs from the second binding condition. In some cases, a system may provide a first probe composition having a first binding condition, and may further provide a second probe composition having a second binding condition, in which a probe of the first probe composition has a substantially identical probe architecture to a probe of the second probe composition, and in which the first binding condition differs from the second binding condition.

A system may further comprise a detection device. A detection device may be configured to detect a signal from a probe when the probe is bound to an analyte. A detection device may be configured to detect signals from probes of a probe composition at single-analyte resolution. A detection device may comprise an optical detector.

In some configurations, a system may comprise a detection device, in which the detection device comprises a sequencing device (e.g., a nucleic acid sequencer, a peptide sequencer). A sequencing device may be utilized to detect a detectable label that can be sequenced, such as a nucleic acid barcode or a peptide barcode. Methods of generating probe binding profiles for characterizing analytes via barcode sequencing are provided in U.S. patent application Ser. No. 19/338,819, which is incorporated by reference in its entirety. A system may comprise a fluidics system, in which the fluidic system is further configured to transfer detectable labels comprising residue sequences to the sequencing device. Detectable labels comprising residue sequences may be transferred from a vessel comprising a plurality of analytes and a probe composition. For example, a fluidic medium comprising the detectable labels comprising residue sequences may be transferred by a fluidics system from a vessel comprising a plurality of analytes to a sequencing device.

A system may comprise a detection device that is configured to scan or read an information tag of a probe library. A detection device for scanning or reading an information tag can include a camera, a QR code scanner, a bar code scanner, or the like. A detection device may be configured to perform one or more steps of: i) scan or read an information tag of a probe library; ii) identify in a database a database entry corresponding to information from the information tag; and iii) retrieve from the database entry information regarding a probe composition of the probe library. Probe composition information stored within the database entry can include spatial location of the probe composition amongst a plurality of vessels of the probe library, concentration of the probe composition, volume of the probe composition. After scanning or reading an information tag of a probe library, a system may be further configured to perform one or more steps of: i) determining a spatial location of a probe composition amongst a plurality of vessels of the probe library, and ii) transferring at least a portion of the probe composition from the spatial location of the probe composition with a fluidics system, as set forth herein.

In addition to the foregoing reagents, also provided herein are kits useful in carrying out the analyses described herein, which kits may include the affinity reagents described above. The kits may optionally include one or more of enrichment reagents used to enrich for low abundance proteins and proteoforms, e.g., beads and antibodies used for the immune-isolation and/or immunoprecipitation of the proteins of interest, wash and other elution reagents, for such enrichment. Such kits may also include the flow-cells and arrays used to immobilize proteins of interest in a single molecule, in an optically detectable format for subsequent analysis in appropriately configured optical detection systems described herein. Such kits can include instructions for carrying out the enrichment, flow-cell deposition, interrogation and follow on analysis of biological samples using such kits.

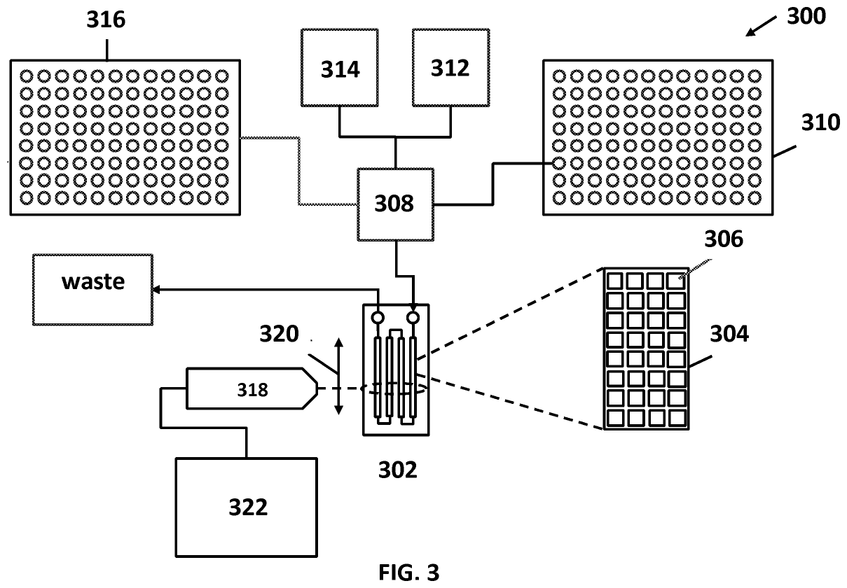

Additionally, provided herein are systems for performing the techniques, reagents, systems, and methods described herein. An example of a system is illustrated in FIG. 3. As shown, the system 300 includes a flowcell 302 that includes an array surface (shown as 304) within the channels of the flow cell upon which individual analyte molecules from a sample may be deposited and immobilized in locations 306 that are individually addressable, and in particular cases are individually optically resolvable from each other using, e.g., fluorescence microscopy or scanning techniques.

The system will also typically include a fluidic delivery system 308 that is configured to deliver different fluids to the flow cell 302 through a series of fluidic lines and utilizing appropriate pumps, valves and other conventional fluid controls. The fluidics system 308 may be fluidically coupled to various sources of fluids and reagents needed to carry out the analysis on the flow cell. For example, as shown, fluidic system 308 is fluidly coupled to a source of a plurality of reagents 310 (shown as a 96 well plate, although any number of different reagent storage systems of varying capacity may be employed) that includes a library of multiple affinity reagents that each have affinity for different characteristics of one or more proteins of interest. Additionally, fluidic system 308 may also be coupled to sources of washing fluids or buffers 312, and removal reagents 314 (for removing bound affinity reagents following detection), as well as any other ancillary fluids and reagents needed for the analysis. Similarly, where flow cells are prepared on the system, the fluidic system may be coupled to sources of different sample materials that are to be analyzed 316 (again, shown as a 96 well plate, although again, any suitable sample storage system or capacity may be suitable).

The reagents sources are typically fluidly connected to the flow-cell using fluidics systems that can separately access different reagents, sample materials and other fluids, and control the timing and volume of different reagents delivered to the flow-cell at different times in order to carry out the deposition, interrogation, washing and removal steps of the analysis process. Such fluidic systems will typically include requisite valves and pumps for carrying out such fluid deliveries and include, for example, those as described in, for example, International Pat. Application No. WO 2023/122589A2, the full disclosure of which is hereby incorporated herein by reference in its entirety for all purposes.

The systems described herein also typically includes a detection system, such as optical detection system 318, for detecting and recording fluorescent signals arising from different positions on the array surface. Such detection systems may generally include line scanning confocal fluorescent microscope systems, which are capable of scanning across large array surfaces (as shown by arrow 320) to detect and record fluorescence across such surfaces at reasonably high scan rates.

The overall systems also typically include one or more computers or processors 322 for controlling the operation of the instrument system including the fluidic system 308 (e.g., to sample different sample sources 316, reagent sources 310 and delivery timing and volume of each), and detection system 318, among other functions, and for recording the detected signals received from the detection system 318, e.g., fluorescent signals, and analyzing such signals to identify potential binding by each of the different affinity reagents. Processors 322 also have access to memory storing instructions that are executed to perform any of the techniques described herein. Included in such memory may be bioinformatic software or firmware that evaluates the signals received and based upon appropriate modeling, identifies likely positive binding events, and then subsequently provides an overall assessment of characteristics of the proteins as described herein including identification information of proteins that are present at any given location on the array and/or the relative abundance of each different protein across the array and ultimately, within the sample being analyzed. Examples of bioinformatic software processes for analyzing such proteoform and proteome data have been described in, for example, U.S. Pat. Nos. 11,545,234, 10,473,654B1, and Egertson, et al., A theoretical framework for proteome-scale single-molecule protein identification using multi-affinity protein binding reagents, bioRxiv, https://doi.org/10.1101/2021.10.11.463967, U.S. Patent Application No. 2022/0236282, International Pat. Application Nos. PCT/US24/15132, and WO 2023/038859. Alternatively, in some cases, recorded data from the binding events, stored as digital information, digital image files, or compressed versions of such image files, may be transmitted to separate servers or cloud-based systems, which house the informatics software that performs this latter analysis and reporting.

The computer system 322 can be an electronic device of a detection system, the electronic device being integral to the detection system or remotely located with respect to the detection system. The computer system 322 includes a computer processing unit (CPU, also “processor” and “computer processor” herein), which can be a single core or multi core processor, or a plurality of processors for parallel processing. The computer system 322 also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. The memory, storage unit, interface and peripheral devices are in communication with the CPU through a communication bus (solid lines), such as a motherboard. The storage unit can be a data storage unit (or data repository) for storing data. The computer system 322 can be operatively coupled to a computer network (“network”) with the aid of the communication interface. The network can be the Internet, an internet and/or extranet, or an intranet and/or extranet that is in communication with the Internet. The network in some cases is a telecommunication and/or data network. The network can include one or more computer servers, which can enable distributed computing, such as cloud computing. For example, one or more computer servers may enable cloud computing over the network (“the cloud”) to perform various aspects of analysis, calculation, and generation of the present disclosure, such as, for example, receiving information of empirical measurements of analytes in a sample; processing information of empirical measurements against a database comprising a plurality of candidate analytes, for example, using a binding model or function set forth herein; generating probabilities of a candidate analytes generating empirical measurements, and/or generating probabilities that extant analytes are correctly identified in the sample, and/or determining abundances of analytes in the sample. Such cloud computing may be provided by cloud computing platforms such as, for example, Amazon Web Services (AWS), Microsoft Azure, Google Cloud Platform, and IBM cloud. The network, in some cases with the aid of the computer system 322, can implement a peer-to-peer network, which may enable devices coupled to the computer system 322 to behave as a client or a server.

The CPU can execute a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback.

The CPU can be part of a circuit, such as an integrated circuit. One or more other components of the system 322 can be included in the circuit. In some cases, the circuit is an application specific integrated circuit (ASIC).

The storage unit can store files, such as drivers, libraries and saved programs. The storage unit can store user data, e.g., user preferences and user programs. The computer system 322 in some cases can include one or more additional data storage units that are external to the computer system 322, such as located on a remote server that is in communication with the computer system 322 through an intranet or the Internet.

The computer system 322 can communicate with one or more remote computer systems through the network. For instance, the computer system 322 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 322 via the network.

Methods as described herein can be implemented by way of machine (e.g., computer processor) executable code stored on an electronic storage location of the computer system 322, such as, for example, on the memory or electronic storage unit. The machine executable or machine readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 322, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 322 can include or be in communication with an electronic display that comprises a user interface (UI) for providing, for example, user selection of algorithms, binding measurement data, candidate proteins, and databases. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit. The algorithm can, for example, receive information of empirical measurements of extant proteins in a sample, compare information of empirical measurements against a database comprising a plurality of protein sequences corresponding to candidate proteins, generate probabilities of a candidate protein generating the observed measurement outcome profile, and/or generate probabilities that candidate proteins are correctly identified in the sample, and/or generate abundances for the proteins in the sample.

The present disclosure provides a non-transitory information-recording medium that has, encoded thereon, instructions for the execution of one or more steps of the methods or techniques set forth herein, for example, when these instructions are executed by an electronic computer in a non-abstract manner. This disclosure further provides a computer processor (i.e. not a human mind) configured to implement, in a non-abstract manner, one or more of the methods set forth herein. All methods, compositions, devices and systems set forth herein will be understood to be implementable in physical, tangible and non-abstract form. The claims are intended to encompass physical, tangible and non-abstract subject matter. Explicit limitation of any claim to physical, tangible and non-abstract subject matter, will be understood to limit the claim to cover only non-abstract subject matter, when taken as a whole. Reference to “non-abstract” subject matter excludes and is distinct from “abstract” subject matter as interpreted by controlling precedent of the U.S. Supreme Court and the United States Court of Appeals for the Federal Circuit as of the priority date of this application.

### Single-Analyte Assays

The present disclosure provides compositions, apparatus and methods for detecting one or more proteins or proteoforms. A protein can be detected using one or more affinity agents having binding affinity for the protein. The affinity agent and the protein can bind each other to form a complex and, during or after formation, the complex can be detected. The complex can be detected directly, for example, due to a label that is present on the affinity agent or protein. In some configurations, the complex need not be directly detected. For example, complex formation can yield a chemical change, such as formation of a nucleic acid tag, that is detected after the complex has been formed and in some cases after the complex has been dissociated.

The present disclosure provides compositions, apparatus and methods that can be useful for characterizing analytes, such as proteins, by obtaining multiple separate and non-identical measurements of the analytes. In particular configurations, the individual measurements may not, by themselves, be sufficiently accurate or specific to make the characterization, but in combination the multiple non-identical measurements can allow the characterization to be made with a high degree of accuracy, specificity and confidence. For example, the multiple separate measurements can include subjecting a sample to reagents that are promiscuous with regard to recognizing a variety of different analytes that are present in the sample. Accordingly, a first measurement carried out using a first promiscuous reagent may perceive a first subset of the analytes without distinguishing different analytes within the subset. A second measurement carried out using a second promiscuous reagent may perceive a second subset of analytes, again, without distinguishing one analyte in the second subset from other analytes in the second subset. However, a comparison of the first and second measurements can distinguish: (i) an analyte that is uniquely present in the first subset but not the second; (ii) an analyte that is uniquely present in the second subset but not the first; (iii) an analyte that is uniquely present in both the first and second subsets; or (iv) an analyte that is uniquely absent in the first and second subsets. The number of promiscuous reagents used, the number of separate measurements acquired, and degree of reagent promiscuity (e.g., the diversity of components recognized by the reagent) can be adjusted to suit the diversity of analytes expected for a particular sample.

The present disclosure provides assays that are useful for detecting one or more analytes. Exemplary assays are set forth herein in the context of detecting proteins. Those skilled in the art will recognize that methods, compositions and apparatus set forth herein can be adapted for use with other analytes such as cells, organelles, nucleic acids, polysaccharides, metabolites, vitamins, hormones, enzyme co-factors, therapeutic agents, candidate therapeutic agents and others set forth herein or known in the art. Particular configurations of the methods, apparatus and compositions set forth herein can be made and used, for example, as set forth in U.S. Pat. Nos. 10,473,654 or 11,282,585; U.S. Pat. App. Pub. Nos. 2020/0082914A1 or 2023/0114905A1; or Egertson et al., BioRxiv (2021), DOI: 10.1101/2021.10.11.463967, each of which is incorporated herein by reference. Exemplary methods, systems and compositions are set forth in further detail below.

A composition, apparatus or method set forth herein can be used to characterize an analyte, or moiety thereof, with respect to any of a variety of characteristics or features including, for example, presence, absence, quantity (e.g., amount or concentration), chemical reactivity, molecular structure, structural integrity (e.g., full length or fragmented), maturation state (e.g., presence or absence of pre- or pro-sequence in a protein), location (e.g., in an analytical system, subcellular compartment, cell or natural environment), association with another analyte or moiety, binding affinity for another analyte or moiety, biological activity, chemical activity or the like. An analyte can be characterized with regard to a relatively generic characteristic such as the presence or absence of a common structural feature (e.g., amino acid sequence length, overall charge or overall pKa for a protein) or common moiety (e.g., a short primary sequence motif or post-translational modification for a protein). An analyte can be characterized with regard to a relatively specific characteristic such as a unique amino acid sequence (e.g., for the full length of the protein or a motif), an RNA or DNA sequence that encodes a protein (e.g., for the full length of the protein or a motif), or an enzymatic or other activity that identifies a protein. A characterization can be sufficiently specific to identify an analyte, for example, at a level that is considered adequate or unambiguous by those skilled in the art.

In particular configurations, a method set forth herein can be used to identify a number of different extant proteins that exceeds the number of affinity reagents used. For example, the number of different protein species identified can be at least 5×, 10×, 25×, 50×, 100× or more than the number of affinity reagents used. This can be achieved, for example, by (1) using promiscuous affinity reagents that bind to multiple different candidate proteins suspected of being present in a given sample, and (2) subjecting the extant proteins to a set of promiscuous affinity reagents that, taken as a whole, are expected to bind each candidate protein in a different combination, such that each candidate protein is expected to generate a unique profile of binding and non-binding events when subjected to the set. Promiscuity of an affinity reagent can arise due to the affinity reagent recognizing an epitope that is known to be present in a plurality of different candidate proteins. For example, epitopes having relatively short amino acid lengths such as dimers, trimers, tetramers or pentamers are expected to occur in a substantial number of different proteins in a typical proteome. Alternatively or additionally, a given promiscuous affinity reagent may recognize multiple different epitopes (e.g., epitopes differing from each other with regard to amino acid composition or sequence). For example, a promiscuous affinity reagent that is designed or selected for its affinity toward a first trimer epitope may also have affinity for a second epitope that has a different sequence of amino acids compared to the first epitope.

Although performing a single binding reaction between a promiscuous affinity reagent and a complex protein sample may yield ambiguous results regarding the identity of the different extant proteins to which it binds, the ambiguity can be resolved by decoding the binding profiles for each extant protein using machine learning or artificial intelligence algorithms that are based on probabilities for the affinity reagents binding to candidate proteins. For example, a plurality of different promiscuous affinity reagents can be contacted with a complex population of extant proteins, wherein the plurality is configured to produce a different binding profile for each candidate protein suspected of being present in the population. The plurality of promiscuous affinity reagents can produce a binding profile for each extant protein that can be decoded to identify a unique combination of positive outcomes (i.e., observed binding events) and/or negative binding outcomes (i.e., observed non-binding events), and this can in turn be used to identify the extant protein as a particular candidate protein having a high likelihood of exhibiting a similar binding profile.

Binding profiles can be obtained for extant proteins and the binding profiles can be decoded or disambiguated to identify extant proteins corresponding to the binding profiles. In many cases one or more binding events produces inconclusive or even aberrant results and this, in turn, can yield ambiguous binding profiles. For example, observation of binding outcomes at single-molecule resolution can be particularly prone to ambiguities due to stochasticity in the behavior of single molecules when observed using certain detection hardware. As set forth above, ambiguity can also arise from affinity reagent promiscuity. Decoding can utilize a binding model that evaluates the likelihood or probability that one or more candidate proteins that are suspected of being present in an assay will have produced an empirically observed binding profile. The binding model can include information regarding expected binding outcomes (e.g., positive binding outcomes and/or negative binding outcomes) for one or more affinity reagents with respect to one or more candidate proteins. A binding model can include a measure of the probability or likelihood of a given candidate protein generating a false positive or false negative binding result in the presence of a particular affinity reagent, and such information can optionally be included for a plurality of affinity reagents. Decoding can be configured to evaluate the degree of compatibility of one or more empirical binding profiles with results computed for various candidate proteins using a binding model. For example, to identify an extant protein in a sample, an empirical binding profile for the extant protein can be compared to results computed by the binding model for many or all candidate proteins suspected to be in the sample. A machine learning or artificial intelligence algorithm can be used. An algorithm used for decoding can utilize Bayesian inference. In some configurations, identity for an extant protein is determined based on a likelihood of the extant protein being a particular candidate protein given the empirical binding pattern or based on the probability of a particular candidate protein generating the empirical binding pattern. Particularly useful decoding methods are set forth, for example, in U.S. Pat. Nos. 10,473,654 or 11,282,585; U.S. Pat. App. Pub. Nos. 2020/0082914A1 or 2023/0114905A1; or Egertson et al., BioRxiv (2021), DOI: 10.1101/2021.10.11.463967, each of which is incorporated herein by reference. It will be recognized that methods set forth herein that are utilized to decode extant proteins may be useful for other analyte identification assays, provided said analyte identification assays provide a binding profile that can be decoded.

In some detection assays, a protein can be cyclically modified and the modified products from individual cycles can be detected. For example, a protein can be sequenced by a sequential process in which each cycle includes steps of detecting the protein and removing one or more terminal amino acids from the protein to produce a shortened protein. The shortened protein is then subjected to subsequent cycles. Optionally, a protein sequencing method can include steps of adding a label to the protein, for example, at the amino terminal amino acid or at the carboxy terminal amino acid. In particular configurations, a method a protein sequencing method can include steps of (i) removing a terminal amino acid from the protein, thereby forming a truncated protein; (ii) detecting a change in signal from the truncated protein, for example, in comparison to the protein prior to truncation; and (iii) identifying the type of amino acid that was removed in step (i) based on the change detected in step (ii). The terminal amino acid can be removed, for example, by removal of one or more amino acids from the amino terminus or carboxyl terminus of the protein. Steps (i) through (iii) can be repeated to produce a series of signal changes that is indicative of the sequence for the protein.

In a first configuration of a protein sequencing method, one or more types of amino acids in the protein can be attached to a label that uniquely identifies the type of amino acid. In this configuration, the change in signal that identifies the amino acid can be loss of signal from the respective label. For example, lysines can be attached to a distinguishable label such that loss of the label indicates removal of a lysine. Alternatively or additionally, other amino acid types can be attached to other labels that are mutually distinguishable from lysine and from each other. For example, lysines can be attached to a first label and cysteines can be attached to a second label, the first and second labels being distinguishable from each other. Exemplary compositions and techniques that can be used to remove amino acids from a protein and detect signal changes are those set forth in Swaminathan et al., Nature Biotech. 36:1076-1082 (2018); or U.S. Pat. Nos.

9,625,469 or 10,545,153, each of which is incorporated herein by reference. Methods and apparatus under development by Erisyon, Inc. (Austin, TX) may also be useful for sequencing, or otherwise detecting, proteins.

In a second configuration of a cyclical protein detection method, a terminal amino acid of a protein can be recognized by an affinity agent that is specific for the terminal amino acid, specific for a labeled terminal amino acid (e.g., the affinity agent can recognize the label alone or in combination with the side chain of a particular type of amino acid). The affinity agent can be detected on the array, for example, due to a label on the affinity agent. Optionally, the label is a nucleic acid barcode sequence that is added to a primer nucleic acid upon formation of a complex. For example, a barcode can be added to the primer via ligation of an oligonucleotide having the barcode sequence or polymerase extension directed by a template that encodes the barcode sequence. The formation of the complex and identity of the terminal amino acid can be determined by decoding the barcode sequence. Multiple cycles can produce a series of barcodes that can be detected, for example, using a nucleic acid sequencing technique. Exemplary affinity agents and detection methods are set forth in U.S. Pat. App. Pub. Nos. 2019/0145982 A1; 2020/0348308 A1; or 2020/0348307 A1, each of which is incorporated herein by reference. Methods and apparatus under development by Encodia, Inc. (San Diego, CA) or Standard BioTools (e.g., technology developed by SomaLogic or Palamedrix) may also be useful for detecting proteins.

In some configurations of the apparatus and methods set forth herein, one or more proteins can be detected on a solid support. For example, protein(s) can be attached to a solid support, the solid support can be contacted with detection agents (e.g., affinity agents) in solution, the agents can interact with the protein(s), thereby producing a detectable signal, and then the signal can be detected to determine the presence of the protein(s). In multiplexed versions of this approach, different proteins can be attached to different addresses in an array, and the probing and detection steps can occur in parallel. In another example, affinity agents can be attached to a solid support, the support can be contacted with proteins in solution, the proteins can interact with the affinity agents, thereby producing a detectable signal, and then the signal can be detected to determine presence, quantity or characteristics of the proteins. This approach can also be multiplexed by attaching different affinity agents to different addresses of an array.

Proteins, affinity agents or other objects of interest can be attached to a solid support via covalent or non-covalent bonds. For example, a linker can be used to covalently attach a protein or other object of interest to an array. A particularly useful linker is a structured nucleic acid particle such as a nucleic acid nanoball (e.g., a concatemeric amplicon produced by rolling circle replication of a circular nucleic acid template) or a nucleic acid origami. For example, a plurality of proteins can be conjugated to a plurality of structured nucleic acid particles, such that each protein-conjugated particle forms a respective address in the array. Exemplary linkers for attaching proteins, or other objects of interest, to an array or other solid support are set forth in U.S. Pat. Nos. 11,203,612 or 11,505,796 or U.S. Pat. App. Pub. No. 2023/0167488 A1, each of which is incorporated herein by reference.

In some configurations of the compositions, apparatus and methods set forth herein, one or more proteins can be present on a solid support, where the proteins can optionally be detected. For example, a protein can be attached to a solid support, the solid support can be contacted with a detection agent (e.g., affinity agent) in solution, the affinity agent can interact with the protein, thereby producing a detectable signal, and then the signal can be detected to determine the presence, absence, quantity, a characteristic or identity of the protein. In multiplexed versions of this approach, different proteins can be attached to different addresses in an array, and the detection steps can occur in parallel, such that proteins at each address are detected, quantified, characterized or identified. In another example, detection agents can be attached to a solid support, the support can be contacted with proteins in solution, the proteins can interact with the detection agents, thereby producing a detectable signal, and then the signal can be detected to determine the presence of the proteins. This approach can also be multiplexed by attaching different probes to different addresses of an array.

In multiplexed configurations, different proteins can be attached to different unique identifiers (e.g., addresses in an array), and the proteins can be manipulated and detected in parallel. For example, a fluid containing one or more different affinity agents can be delivered to an array such that the proteins of the array are in simultaneous contact with the affinity agent(s). Moreover, a plurality of addresses can be observed in parallel allowing for rapid detection of binding events. The total number of proteins of a sample that is detected, characterized or identified can differ from the number of different primary sequences in the sample, for example, due to the presence of multiple copies of at least some protein species. Moreover, the total number of proteins of a sample that is detected, characterized or identified can differ from the number of candidate proteins suspected of being in the sample, for example, due to the presence of multiple copies of at least some protein species, absence of some proteins in a source for the sample, or loss of some proteins prior to analysis.

A protein can be attached to a unique identifier using any of a variety of means. The attachment can be covalent or non-covalent. Exemplary covalent attachments include chemical linkers such as those achieved using click chemistry or other linkages known in the art or described in U.S. patent application Ser. No. 17/062,405, which is incorporated herein by reference. Non-covalent attachment can be mediated by receptor-ligand interactions (e.g., (strept) avidin-biotin, antibody-antigen, or complementary nucleic acid strands), for example, wherein the receptor is attached to the unique identifier and the ligand is attached to the protein or vice versa. In particular configurations, a protein is attached to a solid support (e.g., an address in an array) via a structured nucleic acid particle (SNAP). A protein can be attached to a SNAP and the SNAP can interact with a solid support, for example, by non-covalent interactions of the DNA with the support and/or via covalent linkage of the SNAP to the support. Nucleic acid origami or nucleic acid nanoballs are particularly useful. The use of SNAPs and other moieties to attach proteins to unique identifiers such as tags or addresses in an array are set forth in U.S. Pat. Nos. 11,203,612 and 11,505,796, each of which is incorporated herein by reference.

A method set forth herein can be carried out in a fluid phase or on a solid phase. For fluid phase configurations, a fluid containing one or more proteins can be mixed with another fluid containing one or more affinity agents. For solid phase configurations one or more proteins or affinity agents can be attached to a solid support. One or more components that will participate in a binding event can be contained in a fluid and the fluid can be delivered to a solid support, the solid support being attached to one or more other component that will participate in the binding event. A solid support can be composed of a substrate that is insoluble in aqueous liquid. The substrate can have any of a variety of other characteristics such as being rigid, non-porous or porous. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefins, polyimides etc.), nylon, ceramics, resins, Zeonor™, silica or silica-based materials including silicon and modified silicon, carbon, metals, inorganic glasses, optical fiber bundles, gels, and polymers. In some cases, a solid support may comprise silicon, fused silica, quartz, mica, or borosilicate glass. In particular configurations a flow cell contains the solid support such that fluids introduced to the flow cell can interact with a surface of the solid support to which one or more components of a binding event (or other reaction) is attached.

A method of the present disclosure can be carried out at single analyte resolution. As such, a single analyte (i.e., one and only one analyte), such as a single protein, can be individually manipulated or distinguished using a method set forth herein. A single analyte can be a single molecule (e.g., single protein), a single complex of two or more molecules (e.g., a single protein attached to a structured nucleic acid particle or a single protein attached to an affinity agent), a single particle, or the like. A single analyte may be resolved from other analytes based on, for example, spatial or temporal separation from the other analytes. Reference herein to a ‘single analyte’ in the context of a composition, apparatus or method does not necessarily exclude application of the composition, apparatus or method to multiple single analytes that are manipulated or distinguished individually, unless indicated to the contrary.

A composition, apparatus or method set forth herein can be configured to contact one or more analytes (e.g., an array of different proteins) with a plurality of different affinity agents. For example, a plurality of affinity agents (whether configured separately or as a pool) may include at least 2, 5, 10, 25, 50, 100, 250, 500 or more types of affinity agents, each type of affinity agent differing from the other types with respect to the epitope(s) recognized.

Alternatively or additionally, a plurality of affinity agents may include at most 500, 250, 100, 50, 25, 10, 5, or 2 types of affinity agents, each type of affinity agent differing from the other types with respect to the epitope(s) recognized. Different types of affinity agents in a pool can be uniquely labeled such that the different types can be distinguished from each other. In some configurations, at least two, and up to all, of the different types of affinity agents in a pool may be indistinguishably labeled with respect to each other. Alternatively or additionally to the use of unique labels, different types of affinity agents can be delivered and detected serially when evaluating one or more proteins (e.g., in an array).

An address of an array can contain a single analyte, or it can contain a population of several analytes of the same species (i.e., an ensemble of the analytes). Alternatively, an address can include a population of different analytes. Addresses are typically discrete in an array. Discrete addresses that neighbor each other can be contiguous, or they can be separated by interstitial spaces. An array useful herein can have, for example, addresses that are separated by an average distance of less than 100 microns, 10 microns, 1 micron, 100 nm, 10 nm or less. Alternatively or additionally, an array can have addresses that are separated by an average distance of at least 10 nm, 100 nm, 1 micron, 10 microns, 100 microns or more. The addresses can each have an area of less than 1 square millimeter, 500 square microns, 100 square microns, 10 square microns, 1 square micron, 100 square nm or less. An array can include at least about 1×10⁴, 1×10⁵, 1×10⁶, 1×10⁷, 1×10⁸, 1×10⁹, 1×10¹⁰, 1×10¹¹, 1×10¹², or more addresses.

One or more compositions set forth herein can be present in an apparatus or vessel. For example, a composition of the present disclosure can be present in a vessel, such as a flow cell. As a further option, the vessel can be engaged with a detection apparatus. The vessel can be permanently or temporarily engaged with the detection apparatus. A detection apparatus can be configured to detect contents of a vessel, for example, by acquiring signals arising from the vessel. For example, a detection apparatus can be configured to acquire optical signals through an optically transparent window of the vessel. Optionally, the detection apparatus can be configured for luminescence detection, for example, having an optical train that delivers radiation from an excitation source (e.g., a laser or lamp) then through a window of the vessel. The detection apparatus can further include a camera or other detector that acquires signals transmitted through the window of the vessel and through an optical train. Optionally excitation and emission can be transmitted through the same optical train; however, separate optical trains can also be useful.

A detection apparatus can include a light sensing device that is appropriate for detecting a characteristic set forth herein or known in the art. Particularly useful components of a light sensing device can include, but are not limited to, optical sub-systems or components used in nucleic acid sequencing systems. Examples of useful sub systems and components thereof are set forth in U.S. Pat. App. Pub. No. 2010/0111768 A1 or U.S. Pat. Nos. 7,329,860; 8,951,781 or 9,193,996, each of which is incorporated herein by reference. Other useful light sensing devices and components thereof are described in U.S. Pat. Nos. 5,888,737; 6,175,002; 5,695,934; 6,140,489; or 5,863,722; or U.S. Pat. App. Pub. Nos. 2007/007991 A1, 2009/0247414 A1, or 2010/0111768; or WO2007/123744, each of which is incorporated herein by reference. Light sensing devices and components that can be used to detect luminophores based on luminescence lifetime are described, for example, in U.S. Pat. Nos. 9,678,012; 9,921,157; 10,605,730; 10,712,274; 10,775,305; or 10,895,534, each of which is incorporated herein by reference.

For configurations that use optical detection (e.g., luminescent detection), one or more analytes (e.g., proteins) may be immobilized on a surface, and this surface may be observed by a microscope to detect any signal from the immobilized analytes. The microscope itself may include a digital camera or other luminescence detector configured to record, store, and analyze the data collected during the scan. A luminescence detector can further include an excitation source that is capable of irradiating analytes, for example, proteins at addresses on an array, at an appropriate wavelength. A luminescence detector of the present disclosure can be configured for epiluminescent detection, total internal reflection (TIR) detection, waveguide assisted excitation, or the like. Optical filters or other optical components can be present to tune the wavelength, polarization or other optical properties of excitation and/or emission radiation used by a luminescence detector.

A light sensing device may be based upon any suitable technology, and may be, for example, a charged coupled device (CCD) sensor that generates pixilated image data based upon photons impacting locations in the device. It will be understood that any of a variety of other light sensing devices may also be used including, but not limited to, a detector array configured for time delay integration (TDI) operation, a complementary metal oxide semiconductor (CMOS) detector, an avalanche photodiode (APD) detector, a Geiger-mode photon counter, a photomultiplier tube (PMT), charge injection device (CID) sensors, JOT image sensor (Quanta), or any other suitable detector. Light sensing devices can optionally be coupled with one or more excitation sources, for example, lasers, light emitting diodes (LEDs), arc lamps or other energy sources known in the art.

A light sensing device can be configured for single molecule resolution. For example, waveguides or optical confinements can be used to deliver excitation radiation to locations of a solid support where analytes are located. Zero-mode waveguides can be particularly useful, examples of which are set forth in U.S. Pat. Nos. 7,181,122, 7,302,146, or 7,313,308, each of which is incorporated herein by reference. Analytes can be confined to surface features that function as addresses and facilitate single molecule resolution. For example, analytes can be distributed into wells having nanometer dimensions such as those set forth in U.S. Pat. No. 7,122,482 or 8,765,359, or U.S. Pat. App. Pub. No 2013/0116153 A1, each of which is incorporated herein by reference. The wells can be configured for selective excitation, for example, as set forth in U.S. Pat. No. 8,798,414 or 9,347,829, each of which is incorporated herein by reference. Analytes can be distributed to nanometer-scale posts, such as high aspect ratio posts which can optionally be dielectric pillars that extend through a metallic layer to improve detection of an analyte attached to the pillar. See, for example, U.S. Pat. Nos. 8,148,264, 9,410,887 or 9,987,609, each of which is incorporated herein by reference. Further examples of nanostructures that can be used to detect analytes are those that change state in response to the concentration of analytes such that the analytes can be quantitated as set forth in WO 2020/176793 A1, which is incorporated herein by reference.

A detection apparatus need not be configured for optical detection. For example, an electronic detector can be used for detection of protons or charged labels (see, for example, U.S. Pat. App. Pub. Nos. 2009/0026082 A1; 2009/0127589 A1; 2010/0137143 A1; or 2010/0282617 A1, each of which is incorporated herein by reference in its entirety). A field effect transistor (FET) can be used to detect analytes or other entities, for example, based on proximity of a field disrupting moiety to the FET. The field disrupting moiety can be due to an extrinsic label attached to an analyte or affinity reagent, or the moiety can be intrinsic to the analyte or affinity agent being used. Surface plasmon resonance can be used to detect binding of analytes or affinity agents at or near a surface. Exemplary sensors and methods for attaching molecules to sensors are set forth in U.S. Pat. App. Pub. Nos. 2017/0240962 A1; 2018/0051316 A1; 2018/0112265 A1; 2018/0155773 A1 or 2018/0305727 A1; or U.S. Pat. Nos. 9,164,053; 9,829,456; 10,036,064, each of which is incorporated herein by reference.

Luminescence lifetime can be detected using an integrated circuit having a photodetection region configured to receive incident photons and produce a plurality of charge carriers in response to the incident photons. The integrated circuit can include at least one charge carrier storage region and a charge carrier segregation structure configured to selectively direct charge carriers of the plurality of charge carriers directly into the charge carrier storage region based upon times at which the charge carriers are produced. See, for example, U.S. Pat. Nos. 9,606,058, 10,775,305, and 10,845,308, each of which is incorporated herein by reference. Optical sources that produce short optical pulses can be used for luminescence lifetime measurements. For example, a light source, such as a semiconductor laser or LED, can be driven with a bipolar waveform to generate optical pulses with FWHM durations as short as approximately 85 ps having suppressed tail emission. See, for example, in U.S. Pat. No. 10,605,730, which is incorporated herein by reference.

A detection apparatus can include a fluidics system, for example, configured for fluidic communication with a vessel, such as a flow cell. In some configurations, a detection apparatus can include one or more reservoirs containing affinity reagents or analytes that are delivered to a vessel. Optionally, a detection apparatus can be configured to include a waste receptacle to which waste from the vessel is collected. For example, a composition set forth herein can be delivered from the apparatus through an ingress of a flow cell and waste can be removed through an egress of the flow cell to the apparatus.

One or more compositions set forth herein can be provided in kit form including, if desired, a suitable packaging material. Optionally, one or more compositions can be provided as a solid, such as crystals or a lyophilized pellet. Accordingly, any combination of reagents or components that is useful in a method set forth herein can be included in a kit.

The packaging material included in a kit can include one or more physical structures used to house the contents of the kit. The packaging material can be constructed by well-known methods, preferably to provide a sterile, contaminant-free environment. The packaging materials employed herein can include, for example, those customarily utilized in affinity reagent systems. Exemplary packaging materials include, without limitation, glass, plastic, paper, foil, and the like, capable of holding within fixed limits a component useful in the methods of the present disclosure.

Packaging material or other components of a kit can include a kit label which identifies or describes a particular method set forth herein. For example, a kit label can indicate that the kit is useful for detecting a particular protein, proteoform, or proteome. In another example, a kit label can indicate that the kit is useful for a therapeutic or diagnostic purpose, or alternatively that it is for research use only.

Instructions for use of the packaged reagents or components are also typically included in a kit. The instructions for use can include a tangible expression describing the reagent or component concentration or at least one assay method parameter, such as the relative amounts of kit components and sample to be admixed, maintenance time periods for reagent/sample admixtures, temperature, buffer conditions, and the like.

In some cases, a kit can be configured as a cartridge or component of a cartridge. The cartridge can in turn be configured to be engaged with a detection apparatus. For example, the cartridge can be engaged with a detection apparatus such that contents of the cartridge are in fluidic communication with the detection apparatus or with a flow cell engaged with the detection apparatus. A cartridge can be engaged with a detection apparatus such that contents of the cartridge can be observed by the detection apparatus, for example, using an assay set forth herein.

A solid support or a surface thereof may be configured to display an analyte or a plurality of analytes. A solid support may contain one or more addresses in formed or prepared surfaces. Multiple addresses can be configured to form a pattern. In some cases, a solid support may contain one or more patterned, formed, or prepared surfaces that contain a plurality of addresses, with each address configured to display one or more analytes. Accordingly, an array as set forth herein may comprise a plurality of analytes coupled to a solid support or a surface thereof. In some configurations, a solid support or a surface thereof may be patterned or formed to produce an ordered or repeating pattern of addresses. The deposition of analytes on the repeating pattern of addresses may be controlled by interactions between the solid support and the analytes such as, for example, electrostatic interactions, magnetic interactions, hydrophobic interactions, hydrophilic interactions, covalent interactions, or non-covalent interactions. Accordingly, the coupling of an analyte at each address of an array may produce an array of analytes whose average spacing between analytes is relatively uniform, for example, being determined based upon the tolerance of the ordering or patterning of the solid support and the size of an analyte-binding region for each address. An ordered or patterned array of analytes may be characterized as having a regular geometry, such as a rectangular, triangular, polygonal, or annular grid. In other configurations, a solid support or a surface thereof may have a random or non-repeating pattern of addresses. The deposition of analytes on the random or non-repeating pattern may be controlled by interactions between the solid support and the analytes, or inter-analyte interactions such as, for example, steric repulsion, electrostatic repulsion, electrostatic attraction, magnetic repulsion, magnetic attraction, covalent interactions, or non-covalent interactions.

A solid support or a surface thereof may contain one or more structures or features. A structure or feature may comprise an elevation, profile, shape, geometry, or configuration that deviates from an average elevation, profile, shape, geometry, or configuration of a solid support or surface thereof. A structure or feature may be a raised structure or feature, such as a ridge, post, pillar, or pad, if the structure or feature extends above the average elevation of a surface of a solid support. A structure or feature may be a depressed structure, such as a channel, well, pore, or hole, if the structure or feature extends below the average elevation of a surface of a solid support. A structure or feature may be an intrinsic structure or feature of a substrate (i.e., arising due to the physical or chemical properties of the substrate, or a physical or chemical mechanism of formation), such as surface roughness structures, crystal structures, or porosity. A structure or feature may be formed by a method of processing a solid support. In some configurations, a solid support or a surface may be processed by a lithographic method to form one or more structures or features. A solid support or a surface thereof may be formed by a suitable lithographic method, including, but not limited to photolithography, Dip-Pen nanolithography, nanoimprint lithography, nanosphere lithography, nanoball lithography, nanopillar arrays, nanowire lithography, immersion lithography, neutral particle lithography, plasmonic lithography, scanning probe lithography, thermochemical lithography, thermal scanning probe lithography, local oxidation nanolithography, molecular self-assembly, stencil lithography, laser interference lithography, soft lithography, magnetolithography, stereolithography, deep ultraviolet lithography, x-ray lithography, ion projection lithography, proton-beam lithography, or electron-beam lithography.

A solid support or surface may comprise a plurality of structures or features. Structures or features may be provided as analyte-binding sites for the coupling of analytes or other moieties (e.g., anchoring moieties). A plurality of structures or features may comprise a repeating pattern of structures or features. A plurality of structures or features may comprise a non-ordered, non-repeating, or random distribution of structures or features. A structure or feature may have an average characteristic dimension (e.g., length, width, height, diameter, circumference, etc.) of at least about 1 nanometer (nm), 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1000 nm, or more than 1000 nm. Alternatively or additionally, a structure or feature may have an average characteristic dimension of no more than about 1000 nm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm. An array of structures or features may have an average pitch, in which the pitch is measured as the average separation between respective centerpoints of adjacent structures or features. An array may have an average pitch of at least about 1 nm, 5 nm, 10 nm, 20 nm, 30 nm, 40 nm, 50 nm, 75 nm, 100 nm, 150 nm, 200 nm, 250 nm, 300 nm, 400 nm, 500 nm, 750 nm, 1 micron (μm), 2 μm, 5 μm, 10 μm, 50 μm, 100 μm, or more than 100 μm. Alternatively or additionally, an array may have an average pitch of no more than about 100 μm, 50 μm, 10 μm, 5 μm, 1 μm, 750 nm, 500 nm, 400 nm, 300 nm, 250 nm, 200 nm, 150 nm, 100 nm, 75 nm, 50 nm, 40 nm, 30 nm, 20 nm, 10 nm, 5 nm, 1 nm, or less than 1 nm.

A structure or feature of an array may have a characteristic dimension (e.g., a width, length, or diameter) that is smaller than a characteristic dimension of an analyte or other object (e.g., a nanoparticle) that is attached to the structure or feature. It may be preferable to provide structures or features that are smaller than analytes or other objects attached to the structure or feature to occlude the attachment of additional analytes or other objects to the structure or feature. Alternatively, a structure or feature may have a characteristic dimension that is larger than a characteristic dimension of an analyte or other object (e.g., a nanoparticle) that is attached to the structure or feature.

A solid support or a surface thereof may include a base substrate material and, optionally, one or more additional materials that are contacted or adhered with the substrate material. A solid support may comprise one or more additional materials that are deposited, coated, or inlayed onto the substrate material. Additional materials may be added to the substrate material to alter the properties of the substrate material. For example, materials may be added to alter the surface chemistry (e.g., hydrophobicity, hydrophilicity, non-specific binding, electrostatic properties), alter the optical properties (e.g., reflective properties, refractive properties), alter the electrical or magnetic properties (e.g., dielectric materials, conducting materials, electrically-insulating materials), or alter the heat transfer characteristics of the substrate material. Additional materials contacted or adhered with a substrate material may be ordered or patterned onto the substrate material to, for example, locate the additional material at addresses or locate the additional material at interstitial regions between addresses. Exemplary additional materials may include metals (e.g., gold, silver, copper, etc.), metal oxides (e.g., titanium oxide, silicon dioxide, alumina, iron oxides, etc.), metal nitrides (e.g., silicon nitride, aluminum nitride, boron nitride, gallium nitride, etc.), metal carbides (e.g., tungsten carbide, titanium carbide, iron carbide, etc.), metal sulfides (e.g., iron sulfide, silver sulfide, etc.), and organic moieties (e.g., polyethylene glycol (PEG), dextrans, chemically-reactive functional groups, etc.).

A method of the present disclosure can include the step of coupling one or more analytes to a solid support or a surface thereof, for example, prior to performing a detection step set forth herein. The coupling of one or more analytes to a solid support surface may include covalent or non-covalent coupling of the one or more analytes to the solid support. Covalent coupling of an analyte to a solid support can include direct covalent coupling of an analyte to a solid support (e.g., formation of coordination bonds) or indirect covalent coupling between a reactive functional group of the analyte and a reactive functional group that is coupled to the solid support (e.g., a CLICK-type reaction). Non-covalent coupling can include the formation of any non-covalent interaction between an analyte and a solid support, including electrostatic or magnetic interactions, or non-covalent bonding interactions (e.g., ionic bonds, van der Waals interactions, hydrogen bonding, etc.). The skilled person will readily recognize that the particular analyte and the choice of solid support can affect the selection of a coupling chemistry for the compositions and methods set forth herein.

An array of analytes may be provided for a method, composition, system, or apparatus set forth in the present disclosure. Although analytes are exemplified as proteins throughout the present disclosure, it will be understood that other analytes may be provided in a similar array format. Exemplary analytes include, but are not limited to, cells, organelles, biomolecules, polysaccharides, nucleic acids, lipids, metabolites, hormones, vitamins, enzyme cofactors, therapeutic agents, candidate therapeutic agents, or combinations thereof. An analyte can be a non-biological atom or molecule, such as a synthetic polymer, metal, metal oxide, ceramic, semiconductor, mineral, or a combination thereof.

An array of analytes may be provided on a solid support containing a plurality of discrete analyte-binding sites. The analyte-binding sites may be present at addresses. Each analyte-binding site may be separated from each other analyte-binding site by one or more interstitial regions. For example, each analyte-binding site may be located at a respective address, wherein the addresses are separated from each other by one or more interstitial regions. An array interstitial region may be configured to inhibit binding of analytes or other moieties to the interstitial region, for example by containing a surface coating or layer. Exemplary interstitial region surface layers or coatings can include hydrophobic moieties (e.g., hexamethyldisilazane, alkyl moieties) or hydrophilic moieties (e.g., polyethylene glycol moieties). Surface layers or coatings provided at an interstitial region can comprise linear, branched, or dendrimeric moieties.

A surface layer or coating provided at an interstitial region may be a self-assembled monolayer. An address can include a single analyte-binding site (i.e. one and only one analyte-binding site or, alternatively, a plurality of analyte-binding sites can be present at a given address.

Array analyte-binding sites can comprise one or more moieties that are coupled or otherwise bound to a solid support at the analyte-binding site. Moieties may be bound to a solid support at an analyte-binding site for facilitating coupling of an analyte to the analyte-binding site, or to inhibit unwanted binding of moieties to the analyte-binding site. Moieties may be covalently or non-covalently bound to a solid support at an analyte-binding site.

An analyte-binding site may be provided with one or more moieties that couple an analyte to the analyte-binding site. Coupling moieties can include non-covalent coupling moieties (e.g., oligonucleotides, receptor-ligand binding pairs, electrically-charged moieties, magnetic moieties, etc.), or covalent coupling moieties (e.g., Click-type reactive groups, etc.). An analyte-binding site may be provided with one or more passivating moieties that inhibit unwanted or unexpected binding of moieties to the analyte-binding site. Exemplary passivating moieties can include polymeric molecules such as polyethylene glycol (PEG), bovine serum albumin, pluronic F-127, polyvinylpyrrolidone, and Teflon, or hydrophobic materials such as hexamethyldisilazane. A passivating moiety may be covalently or non-covalently bound to a solid support at an analyte-binding site. An analyte-binding site may contain a covalently bound passivating moiety and a non-covalently bound passivating moiety. For example, an analyte-binding site may contain a PEG moiety that is covalently attached to the solid support at the analyte-binding site and a bovine serum albumin moiety that is electrostatically bound to the analyte-binding site.

Analyte-binding sites may have an average characteristic dimension of at least about 10 nm, 25 nm, 50 nm, 100 nm, 150 nm, 200 nm, 250 nm, 300 nm, 500 nm, 1 μm, or more than 1 μm. Alternatively or additionally, analyte-binding sites may have an average characteristic dimension of no more than about 1 μm, 500 nm, 300 nm, 250 nm, 200 nm, 150 nm, 100 nm, 50 nm, 25 nm, 10 nm, or less than 10 nm.

Analytes may be attached directly to analyte-binding sites, for example, by coupling of a moiety attached to an analyte to a moiety attached to an analyte-binding site. Alternatively, analytes may be attached to analyte-binding sites by an anchoring moiety. An anchoring moiety may attach an analyte to an analyte-binding site, and optionally orient the analyte and/or occlude additional analytes from attaching to the analyte-binding site. An anchoring moiety may comprise a nanoparticle, such as a metal nanoparticle, a metal oxide nanoparticle, a semiconductor nanoparticle, a carbon nanoparticle, or a polymeric nanoparticle. Preferably, an anchoring moiety may comprise a nucleic acid nanoparticle. A nucleic acid nanoparticle of an anchoring moiety may comprise a first face containing one or more coupling moieties, and a second face containing an analyte-coupling site. The first face and the second face of the anchoring moiety may be substantially opposed. The anchoring moiety may further comprise a linking moiety that attaches the analyte to the anchoring moiety. The linking moiety may spatially separate the analyte from the surface of the array, for example by a distance of at least about 5 nm, 10 nm, 15 nm, 20 nm, 25 nm, 30 nm, 40 nm, 50 nm, or more than 50 nm. The linking moiety may comprise a flexible linker (e.g., a PEG or alkyl moiety) or a rigid linker (e.g., a double-stranded nucleic acid linker). An anchoring moiety may be attached to one and only one analyte. An anchoring moiety may be attached to more than one analyte. Additional aspects of anchoring moieties are described in U.S. Pat. Nos. 11,203,612, and 11,505,796, each of which is incorporated herein by reference in its entirety.

It may be especially useful to provide an array of analytes with a diversity of polypeptide species. The diversity of polypeptide species may be measured with respect to a proteome, sub-proteome (e.g., a tissue proteome, a cell proteome, an organelle proteome, a metabolome, a signalome, an albuminome, etc.), or a microbiome. An array of analytes may be provided with a diversity of polypeptide species as measured by total number of polypeptide species, percentage of species of a proteome, subproteome, or microbiome, number of proteoforms of a polypeptide species, or polypeptide dynamic range.

An array of analytes may be provided with more than one unique species of polypeptide. A first polypeptide may be considered unique from a second polypeptide if the amino acid sequences of the first polypeptide and second polypeptide differ. An array of analytes may be provided with at least about 2, 5, 10, 50, 100, 500, 1000, 2000, 5000, 10000, 15000, 20000, 25000, 30000, 40000, 500000, 100000, or more than 100000 unique species of polypeptides. Alternatively or additionally, an array of analytes may be provided with no more than about 100000, 50000, 40000, 30000, 25000, 20000, 15000, 10000, 5000, 2000, 1000, 500, 100, 50, 10, 5, 2, or less than 2 unique species of polypeptides.

An array of analytes may be provided with a fraction or percentage of species of a proteome, subproteome, or microbiome. An array of analytes may be provided with at least about 0.1%, 1%, 5%, 10%, 15%, 20%, 25%, 30%, 35%, 40%, 45%, 50%, 55%, 60%, 65%, 70%, 75%, 80%, 85%, 90%, 95%, 99%, 99.9%, or more than 99.9% of polypeptide species of a proteome, subproteome, or microbiome. Alternatively or additionally, an array of analytes may be provided with no more than about 99.9%, 99%, 95%, 90%, 85%, 80%, 75%, 70%, 65%, 60%, 55%, 50%, 45%, 40%, 35%, 30%, 25%, 20%, 15%, 10%, 5%, 1%, 0.1%, or less than 0.1% of polypeptide species of a proteome, subproteome, or microbiome.

An array of analytes may be provided with more than one proteoform of a polypeptide species. An array of analytes may be provided with more than one proteoform for two or more unique polypeptide species. Types of proteoforms of a polypeptide species can include coding variation proteoforms, translational variation proteoforms, post-translational modification proteoforms, splice variants, and combinations thereof. An array of analytes may be provided with at least about 2, 3, 4, 5, 10, 20, 50, 100, 200, 500, 1000, or more than 1000 proteoforms of a polypeptide species. Alternatively or additionally, an array of analytes may be provided with no more than about 1000, 500, 200, 100, 50, 20, 10, 5, 4, 3, or less than 3 proteoforms of a polypeptide species.

An array of analytes may be provided with a dynamic range of polypeptides. Dynamic range can refer to the ratio of abundance between a more populous polypeptide species and a less populous polypeptide species. A dynamic range can be an absolute measure (ratio of most populous polypeptide species to least populous polypeptide species) or a relative measure (ratio of a first particular polypeptide species to a second particular polypeptide species). An array of analytes may be provided with a dynamic range of at least about 10, 10², 10³, 10⁴, 10⁵, 10⁶, 10⁷, 10⁸, 10⁹, 10¹⁰, 10¹¹, 10¹², or more than 10¹². Alternatively or additionally, an array of analytes may be provided with a dynamic range of no more than about 10¹², 10¹¹, 10¹⁰, 10⁹, 10⁸, 10⁷, 10⁶, 10⁵, 10⁴, 10², 10², 10, or less than 10.

In some methods, providing an array of analytes may further comprise forming the array of analytes. An array of analytes may be formed by a process that includes a step of coupling analytes to analyte-binding sites of the array. An analyte may be coupled to an analyte-binding site by coupling of a coupling moiety attached to the analyte to a compatible coupling moiety attached to the analyte-binding site. In some cases where an analyte is attached to an anchoring moiety, a step of coupling the analyte to the analyte-binding site may comprise coupling the anchoring moiety to the analyte-binding site. In particular cases, an analyte may be coupled to an analyte-binding site by coupling of a coupling moiety attached to an anchoring moiety to a compatible coupling moiety attached to the analyte-binding site.

When forming an array of analytes, a plurality of analytes may be provided in a fluidic medium. A fluidic medium containing a plurality of analytes may be contacted to a solid support comprising a plurality of analyte-binding sites. After contacting the fluidic medium comprising the analytes to the solid support, analytes may couple to analyte-binding sites, thereby forming the array of analytes. In some cases, after contacting a fluidic medium containing analytes to a solid support containing analyte-binding sites, a mass transfer process may occur to facilitate coupling of the analytes to the analyte-binding sites. A mass transfer process can include chemical or mechanical processes that increase a rate of mass transfer of analytes to the surface of the solid support containing the analyte-binding sites. Chemical methods can include altering a pH (e.g., increasing the pH, decreasing the pH), ionic strength (e.g., increasing the ionic strength, decreasing the ionic strength), or temperature (e.g., increasing the temperature, decreasing the temperature) of a fluidic medium containing analytes. A chemical method of increasing mass transfer of analytes may depend upon the chemical composition of the analytes or moieties attached thereto (e.g., anchoring moieties). For example, an analyte attached to a nucleic acid nanoparticle (or any other particle having a net negative electrical surface charge) may transfer toward a hydrophobic surface more readily if the ionic strength of the fluidic medium is decreased. Mechanical methods of increasing mass transfer can include any suitable method of imparting a force on an analyte or a moiety attached thereto, such as centrifugation, electrophoresis, or magnetic attraction. Accordingly, it may be useful to provide an analyte attached to an electrically-charged particle, a magnetic particle, a particle that is denser than a fluidic medium, or a combination thereof.

A method of forming an array of analytes may include repeating one or more steps of attaching analytes to analyte-binding sites of the array. It may be preferable to repeat certain analyte-coupling steps to increase the analyte-binding site occupancy of an array of analytes. Fluidic media containing analytes may be repetitively or sequentially contacted to a solid support. A method of forming an array of analytes may further include a rinsing step (e.g., after contacting a fluidic medium to a solid support), thereby removing unbound or weakly-bound analytes or other moieties (e.g., anchoring moieties) from contact with the solid support.

It may be useful to provide an array of analytes with one or more fiducial elements. A fiducial element may comprise a detectable address or region of the array that facilitates spatial identification on the array. A fiducial element may provide a landmark or fixed reference for determining position on an array, a measurement of length or distance on the array, a spatial reference for calibrating a detection device (e.g., a sensor or camera), and a spatial reference for registering the addresses of analyte-binding sites consistently over the timespan of an array-based process. In some cases, fiducial elements may be disposed in interstitial regions of a solid support. In other cases, fiducial elements may be disposed at analyte-binding sites of the solid support.

An analyte or affinity reagent can be attached to a retaining component such as a particle, array address, solid support or other substance. A particularly useful retaining component is a structured nucleic acid particle (SNAP). SNAPs can optionally include nucleic acid origami. A nucleic acid origami can include one or more nucleic acids folded into a variety of overall shapes such as a disk, tile, cylinder, cone, sphere, cuboid, tubule, pyramid, polyhedron, or combination thereof. Examples of structures formed with DNA origami are set forth in Zhao et al. Nano Lett. 11, 2997-3002 (2011); Rothemund Nature 440:297-302 (2006); Sigle et al, Nature Materials 20:1281-1289 (2021); or U.S. Pat. No. 8,501,923 or 9,340,416, each of which is incorporated herein by reference. In some configurations, a structured nucleic acid particle can include a nucleic acid nanoball and the nucleic acid nanoball can include a concatemeric repeat of amplified nucleotide sequences. The concatemeric amplicons can include complements of a circular template amplified by rolling circle amplification. Exemplary nucleic acid nanoballs and methods for their manufacture are described, for example, in U.S. Pat. No. 8,445,194, which is incorporated herein by reference. Further examples of structured nucleic acid particles are set forth in U.S. Pat. Nos. 11,203,612 or 11,505,796; or U.S. Pat. App. Pub. Nos. 2022/0162684 A1 or 2023/0167488 A1, each of which is incorporated herein by reference.

A retaining component, such as a SNAP, may have any of a variety of sizes and shapes to accommodate use in a desired application. For example, a retaining component can have a regular or symmetric shape or, alternatively, it can have an irregular or asymmetric shape. The shape can be rigid or pliable. The size or shape of a SNAP or other retaining component can be characterized with respect to length, area (i.e. footprint), or volume. The size or shape of a SNAP or other retaining component can be smaller than an address in an array to which it will associate or attach. Optionally, the relative sizes and shapes of an individual retaining component and an address to which it will attach are configured to preclude more than one of the retaining components from occupying the address.

Optionally, a retaining component (e.g., SNAP) or population thereof has a minimum, maximum or average length of at least about 50 nm, 100 nm, 250 nm, 500 nm, 1 micron, 5 micron or more. Alternatively or additionally, a retaining component (e.g., SNAP) or population thereof has a minimum, maximum or average length of no more than about 5 micron, 1 micron, 500 nm, 250 nm, 100 nm, 50 nm, or less.

Optionally, a retaining component (e.g., SNAP) or population thereof has a minimum, maximum or average volume of at least about 1 micron³, 10 micron³, 100 micron³, 1 mm³ or more. Alternatively or additionally, a retaining component (e.g., SNAP) or population thereof has a minimum, maximum or average volume of no more than about 1 mm³, 100 micron³, 10 micron³, 1 micron³ or less.

Optionally, the minimum, maximum or average area (i.e. footprint) for a retaining component (e.g., SNAP) is at least about 10 nm², 100 nm², 1 micron², 10 micron², 100 micron², 1 mm² or more. Alternatively or additionally, the minimum, maximum or average area for a retaining component (e.g., SNAP) footprint is at most about 1 mm², 100 micron², 10 micron², 1 micron², 100 nm², 10 nm², or less. The footprint of a retaining component (e.g., SNAP) may have a regular shape or an approximately regular shape, such as triangular, square, rectangular, circular, ovoid, or polygonal shape.

A structured nucleic acid particle (e.g., having origami or nanoball structures) may include regions of single-stranded nucleic acid, regions of double-stranded nucleic acid, or combinations thereof. For example, a SNAP can have a nucleic acid origami structure which includes a scaffold strand and a plurality of staple strands. The scaffold strand can be configured as a single, continuous strand of nucleic acid, and the staples can be formed by nucleic acid strands that hybridize, in whole or in part, with the scaffold strand.

A structured nucleic acid particle (e.g., nucleic acid origami, or nucleic acid nanoball) may be formed by an appropriate technique including, for example, those known in the art. Nucleic acid origami can be designed, for example, as described in Rothemund, Nature 440:297-302 (2006), or U.S. Pat. No. 8,501,923 or 9,340,416, each of which is incorporated herein by reference. Nucleic acid origami may be designed using a software package, such as CADNANO (cadnano.org), ATHENA (github.com/lcbb/athena), or DAEDALUS (daedalus-dna-origami.org).

Other useful retaining components include artificial polymers. Artificial polymers can include polymers that are made by human activity rather than occurring naturally. For example, a polymer that is made at least in part by human activity or that includes at least one artificial moiety is referred to as an “artificial polymer.” In some cases the artificial polymers are configured as dendrons. A dendron will include at least one branched chain polymer.

Particularly useful artificial polymers include, for example, poly (amidoamine) (PAMAM) dendrimer, poly (amidoamine) dendron, hyperbranched polymers such as linear and branched polyethyleneimine (PEI) and polypropyleneimine (PPI), star polymers, grafted polymers, peptide-based linear or branched dendrimers such as branched poly-L-lysine (PLL) and silane-cored dendrimer. Other useful artificial polymers include dendrimer nucleic acids having branching structures. See, for example, Liu et al., J. Mater. Chem. B 9:4991-5007 (2021) and Meng et al., ACS Nano 8:6171-6181 (2014), each of which is incorporated herein by reference. Examples of useful polymers are set forth in Tomalia, et al. J Polym Sci Part A: Polym Chem 40:2719-2728 (2002); Higashihara, et al. Polym J 44, 14-29 (2012); Gupta, et al. J. Phys. Chem. B 124, 20, 4193-4202 (2020); Ren, et al. Chem. Rev. 116, 12, 6743-6836 (2016); Chis, et al. Molecules 25(17):3982 (2020); Zheng, et al. or Chem. Soc. Rev. 44, 4091-4130 (2015), each of which is incorporated herein by reference.

The present disclosure provides compositions and methods for improving binding of analytes to affinity reagents by increasing avidity of the binding interaction. In particular embodiments, avidity between an analyte and affinity reagent can be increased by association of a docker with the analyte and association of a tether with the affinity reagent. The docker and tether recognize each other and can thus bind to each other. Avidity of the interaction between the affinity reagent and analyte is a function not only of recognition between the paratope and epitope, but also recognition between the docker and tether.

A docker can be associated with an analyte via covalent and/or non-covalent attachment of the docker to the analyte. Similarly, a tether can be associated with an affinity reagent via covalent and/or non-covalent attachment of the docker to the affinity reagent. Exemplary attachment chemistries include those set forth herein in the context of attaching analytes and affinity reagents to retaining components, addresses of an array, solid supports, labels, etc. In some configurations, a docker or tether can be attached to a particle (e.g., structured nucleic acid particle), unique identifier, address or solid support to which an analyte or affinity reagent, respectively, is attached.

Accordingly, the present disclosure provides a method of processing an analyte. The method can include the steps of (a) providing an analyte comprising an epitope and a docker; (b) providing an affinity reagent, wherein the affinity reagent comprises a paratope that recognizes the epitope and a tether that recognizes the docker; and (c) contacting the analyte with the affinity reagent, whereby the affinity reagent associates with the analyte via binding of the paratope to the epitope and via binding of the tether to the docker. Optionally, the method further includes a step of detecting association of the affinity reagent with the analyte, thereby identifying the analyte. In another option, the analyte is present in a sample including other analytes and the method further includes a step of separating the analyte from the other analytes via the association of the affinity reagent with the analyte.

Compositions set forth herein can interact with each other via covalent bonds. Molecules, moieties thereof or atoms thereof can form covalent bonds with other molecules, moieties or atoms. Covalent interactions can be reversible or irreversible in the context of a method set forth herein. A covalent bond can arise due to a chemical reaction between a first reactive moiety and a second reactive moiety, optionally in the presence of a third intermediary or catalytic moiety. Covalent bonds can be formed via various chemical mechanisms, including addition, substitution, elimination, oxidation, and reduction. In some cases, a covalent binding interaction may be formed by a Click-type reaction, as set forth herein (e.g., methyltetrazine (mTz)-tetracyclooctylene (TCO), azide-dibenzocyclooctene (DBCO), thiol-epoxy). In some cases, a ligand-receptor-type binding interaction can form a covalent binding interaction. For example, SpyCatcher-SpyTag, SnoopCatcher-SnoopTag, and SdyCatcher-SdyTag are receptor-ligand binding pairs that can form covalent binding interactions due to isopeptide bond formation. Additional useful covalent binding interactions can include coordination bond formation, such as between a metal-containing substrate and a ligand. Exemplary coordination bonds can include silicon-silane, metal oxide-phosphate, and metal oxide-phosphonate. Useful reagents and mechanisms for forming covalent binding interactions, including bioorthogonal binding interactions, as set forth herein, are provided in U.S. Pat. Nos. 11,203,612 or 11,505,796, each of which is herein incorporated by reference in its entirety.

Compositions set forth herein can interact with each other via non-covalent bonds. A non-covalent bond can include an electrostatic or magnetic interaction between a first moiety and a second moiety. A non-covalent bond can include electrostatic interactions such as ionic bonding, hydrogen bonding, halogen bonding, Van der Waals interactions, Pi-Pi stacking, Pi-ion interactions, Pi-polar interactions, or magnetic interactions. In some cases, a non-covalent bond may be formed by hybridization of a first oligonucleotide to a complementary second oligonucleotide. Such bonding is also known as Watson-Crick base-pairing. In some cases, a non-covalent interaction may be formed by a receptor-ligand binding pair, such as streptavidin-biotin. Other useful non-covalent interactions can include affinity reagent-target interactions, such as antibody-epitope or aptamer-epitope interactions.

Systems and methods for forming and utilizing arrays, such as those set forth herein, may contain multiple types of covalent and/or non-covalent interactions. For example, a useful array site configuration may comprise an analyte (e.g., a polypeptide) that is covalently bonded to an oligonucleotide, in which the oligonucleotide is hybridized to a nucleic acid nanoparticle, in which the nucleic acid nanoparticle is hybridized to a surface-coupled oligonucleotide, and in which the surface-coupled oligonucleotide is covalently bonded to a surface of a solid support. This example may be extended to further include an affinity reagent that is non-covalently bound to the analyte. The affinity reagent bound to the analyte, in turn, may be covalently bonded to a nanoparticle or a moiety thereof (e.g., an oligonucleotide). The skilled person will recognize that the various covalent and non-covalent interactions occurring in the system and methods set forth herein may vary with respect to both time-scale and reversibility (or lack thereof) for association and/or dissociation of the binding interactions. Accordingly, it will be recognized that certain binding interactions (e.g., covalent binding of an analyte to an oligonucleotide) will be selected to inhibit or minimize a likelihood of association or dissociation over the duration of a method, or a step thereof, as set forth herein, and other binding interactions (e.g., non-covalent binding of an affinity reagent to an analyte) will be selected to facilitate or increase a likelihood of association or dissociation within the duration of a method or a step thereof, as set forth herein.

Entities, such as affinity reagents and their binding targets, can be associated with each other and dissociated form each other in a method set forth herein. Association of a first entity to a second entity can involve a contacting step, in which the first entity is brought into proximity of the second entity, and an association step in which a first coupling moiety of the first entity forms a binding interaction with a second coupling moiety of the second entity. Dissociation of a first entity and a second entity need not be construed as a reversal of an association process between the first entity and the second entity. For example, a first entity comprising a first oligonucleotide coupled to a second entity comprising a second oligonucleotide by hybridization of the first oligonucleotide to the second oligonucleotide could be dissociated by dehybridization of the nucleic acids (thereby returning the first entity and the second entity as originally provided before association), or dissociated by enzymatic cleavage of the hybridized nucleic acids (thereby providing the first and the second entities with each individually further comprising an at least partially double-stranded cleavage product).

Systems or methods set forth herein may utilize one or more fluidic media to implement a process or step thereof. For array-based processes and systems, fluidic media may be provided for various process steps, including preparing arrays, attaching analytes to arrays, associating affinity agents to analytes, dissociating affinity agents from analytes, rinsing unbound moieties from array surfaces, performing detection processes on arrays, displacing a fluidic medium from contact with an array or other system components, and various other chemical and/or physical alterations of analytes or array components. A fluidic medium may be formulated to deliver a plurality of macromolecules (e.g., analytes, affinity agents) to an array as set forth herein. A fluidic medium may be formulated to mediate an interaction between macromolecules (e.g., an interaction between an analyte and an affinity agent).

A fluidic medium may be a single-phase or multi-phase fluidic medium. A multi-phase fluidic medium can include a gas phase and a liquid phase or at least two immiscible liquids. A multi-phase fluidic medium may comprise an interface between a first phase and a second phase. An interface between two fluidic phases may be laminar (e.g., an oil phase floating on an aqueous phase) or dispersed (e.g., bubbles, vesicles or droplets). A dispersed interface may be formed by a process such as emulsification. A divided interface may be stable (e.g., an emulsion) or unstable (e.g., a flocculating suspension). A multi-phase fluidic medium may comprise a colloidal agent that mediates an interface between a first phase and a second phase.

A fluidic medium can further contain solids, including particles (e.g., microparticles, nanoparticles). A fluidic medium comprising solids may be provided as a mixture, a suspension, or a slurry. It may be advantageous to provide a fluidic medium comprising a mixture or suspension of macromolecules. In some cases, solubility or suspendability of solids, such as particles or macromolecules, within a fluidic medium can be modulated by the composition of the fluidic medium. For example, alteration of fluidic properties such as solvent composition, ionic strength, and/or pH can induce precipitation, sedimentation, or flocculation of solvated or suspended solids.

A fluidic medium may be formulated with any one of numerous components depending upon its intended application. A fluidic medium can comprise one or more solvents. A single-phase fluidic medium can comprise two or more miscible solvents. In a mixture of miscible solvents, a solvent may be considered a base solvent if it comprises a greater than 50% fraction on a mass, molar, or volumetric basis. A miscible solvent may be mixed into a base solvent to alter a physical property of the base solvent, such as polarity, density, pH, viscosity, or surface tension. A fluidic medium can comprise a polar solvent or a non-polar solvent. A fluidic medium can comprise a protic or aprotic solvent. A fluidic medium can comprise an aqueous medium. A fluidic medium can comprise an organic solvent, such as acetic acid, acetone, acetonitrile, benzene, a butanol, 2-butanone, carbon tetrachloride, chlorobenzene, chloroform, cyclohexane, 1,2-dichloroethane, diethylene glycol, diethyl ether, diglyme, 1,2-dimethoxy-ethane, dimethylformamide, dimethyl sulfoxide, 1,4-dioxane, ethanol, ethyl acetate, ethylene glycol, glycerin, heptane, hexamethylphosphoramide, hexamethylphophorus triamide, hexanes, methanol, methyl t-butyl ether, methylene chloride, N-methyl-pyrrolidinone, nitromethane, pentane, petroleum ether, 1-proponal, 2-propanol, pyridine, tetrahydrofuran, toluene, triethyl amine, xylene, or a combination thereof. A fluidic medium can comprise a polar solvent, such as N-methyl pyrrolidone, tetrahydrofuran, ethyl acetate, acetone, dimethylfuran, acetonitrile, dimethyl sulfoxide, propylene carbonate, N-butanol, isopropyl alcohol, nitromethane, ethanol, methanol, acetic acid, or a combination thereof. A fluidic medium can comprise a non-polar solvent, such as benzene, carbon tetrachloride, chloroform, cyclohexane, dichloromethane, dimethoxyethane, ethyl ether, heptane, hexachloroethane, hexane, limonene, naphtha, pentane, tetrachloroethylene, tetrahydrofuran, toluene, xylenes, and combinations thereof. In some cases, a fluidic medium may comprise an aprotic solvent, such as N-methyl pyrrolidone, tetrahydrofuran, ethyl acetate, acetone, dimethylfuran, acetonitrile, dimethyl sulfoxide, propylene carbonate, or a combination thereof.

A fluidic medium may further comprise one or more components, including: 1) an ionic species, 2) a buffering agent, 3) a surfactant or detergent, 4) a chelating agent, 5) a denaturing agent or a chaotrope, 6) a cosmotropic or crowding agent, 7) a clouding agent, 8) a reactive scavenger, and 9) a blocking agent.

A fluidic medium may comprise one or more ionic species. An ionic species may be provided to a fluidic medium as a salt, thereby providing an anionic species and a cationic species to the fluidic medium. An ionic species can include a zwitterionic species. A fluidic medium may comprise a cationic species such as Nat, K⁺, Ag⁺, Cu⁺, NH⁴⁺, Mg²⁺, Ca²⁺, Cu²⁺, Cd²⁺, Zn²⁺, Fe²⁺, Co²⁺, Ni²⁺, Cr²⁺, Mn²⁺, Ge²⁺, Sn²⁺, Al³⁺, Cr³⁺, Fe³⁺, Co³⁺, Ni³⁺, Ti³⁺, Mn³⁺, Si⁴⁺, V⁴⁺, Ti⁴⁺, Mn⁴⁺, Ge⁴⁺, Se⁴⁺, V⁵⁺, Mn⁵⁺, Mn⁶⁺, Se⁶⁺, and combinations thereof. A fluidic medium may comprise an anionic species such as F⁻, Cl⁻, Br⁻, ClO₃⁻, H₂PO₄⁻, HCO₃⁻, HSO₄⁻, OH⁻, I⁻, NO₃⁻, NO₂⁻, MnO₄⁻, SCN⁻, CO₃²⁻, CrO₄²⁻, Cr₂O₇²⁻, HPO₄²⁻, SO₄²⁻, SO₃²⁻, PO₄³⁻, and combinations thereof. A fluidic medium may comprise a chelating agent, such as ethylenediaminetetraacetic acid (EDTA), nitrilotriacetic acid, n-hydroxyethylenediaminetetraacetic acid (HEDTA), oxalic acid, malic, acid, rubeanic acid, citric acid, or combinations thereof.

A fluidic medium may include a buffering species including, but not limited to, MES, Tris, Bis-tris, Bis-tris propane, ADA, ACES, PIPES, MOPSO, MOPS, BES, TES, HEPES, HEPBS, HEPPSO, DIPSO, MOBS, TAPSO, TAPS, TABS, POPSO, TEA, EPPS, Tricine, Gly-Gly, Bicine, AMPD, AMPSO, AMP, CHES, CAPSO, CAPS, PBS, and CABS.

A fluidic medium may comprise a surfactant or detergent. A surfactant or detergent may comprise a cationic surfactant or detergent, an anionic surfactant or detergent, a zwitterionic surfactant or detergent, an amphoteric surfactant or detergent, or a non-ionic surfactant or detergent. A fluidic medium may include a surfactant species including, but not limited to, stearic acid, lauric acid, oleic acid, sodium dodecyl sulfate, sodium dodecyl benzene sulfonate, dodecylamine hydrochloride, hexadecyltrimethylammonium bromide, polyethylene oxide, nonylphenyl ethoxylates, Triton X, pentapropylene glycol monododecyl ether, octapropylene glycol monododecyl ether, pentaethylene glycol monododecyl ether, octaethylene glycol monododecyl ether, lauramide monoethylamine, lauramide diethylamine, octyl glucoside, decyl glucoside, lauryl glucoside, Tween 20, Tween 80, n-dodecyl-β-D-maltoside, nonoxynol 9, glycerol monolaurate, polyethoxylated tallow amine, poloxamer, digitonin, zonyl FSO, 2,5-dimethyl-3-hexyne-2,5-diol, Igepal CA630, Aerosol-OT, triethylamine hydrochloride, cetrimonium bromide, benzethonium chloride, octenidine dihydrochloride, cetylpyridinium chloride, adogen, dimethyldioctadecylammonium chloride, CHAPS, CHAPSO, cocamidopropyl betaine, amidosulfobetaine-16, lauryl-N,N-(dimethylammonio) butyrate, lauryl-N,N-(dimethyl)-glycinebetaine, hexadecyl phosphocholine, lauryldimethylamine N-oxide, lauryl-N,N-(dimethyl)-propanesulfonate, 3-(1-pyridinio)-1-propanesulfonate, 3-(4-tert-butyl-1-pyridinio)-1-propanesulfonate, N-laurylsarcosine, and combinations thereof.

A fluidic medium may comprise a denaturing or chaotropic species, such as acetic acid, trichloroacetic acid, sulfosalicylic acid, sodium bicarbonate, ethanol, ethylenediamine tetraacetic acid (EDTA), urea, guanidinium chloride, lithium perchlorate, sodium dodecyl sulfate, 2-mercaptoethanol, dithiothreitol, tris(2-carboxyethyl)phosphine (TCEP), or a combination thereof. A denaturing or chaotropic species may be provided to alter a conformational state of an array component (e.g., causing denaturation of a polypeptide), or may be provided to maintain a conformational state of an array component (e.g., maintaining a polypeptide in a denatured or partially-denatured state).

A fluidic medium may comprise a cosmotropic species, such as carbonate ion, sulfate ion, phosphate ion, magnesium ion, lithium ion, zinc ion, aluminum ion, trehalose, glucose, proline, tert-butanol, or a combination thereof. A fluidic medium may comprise a clouding agent such as sodium chloride, potassium chloride, sodium bromide, potassium bromide, sodium nitrate, sodium sulfate, sodium phosphate, or a combination thereof. A cosmotropic species may be provided to decrease a separation distance between molecules and array components (e.g., causing smaller separation between an affinity agent and an analyte).

A fluidic medium may comprise a reactive scavenger species. A reactive scavenger may be provided to reduce solution-phase concentrations of reactive species (e.g., oxidizing or reducing species). A reactive scavenger may be provided during a photon-mediated process (e.g., fluorescent imaging) to reduce photodamage or other deleterious photon-related processes (e.g., singlet oxygen generation, free radical generation). Exemplary reactive scavenger species can include ascorbic acid, 9,10-anthracenediyl-bis(methylene) dimalonic acid (ABDA), epigallocatechin gallate (EPGG), N-acetyl-L-cysteine, caffeic acid, reseveratrol, 4-hydroxy-2,2,6,6-tetramethylpiperidin-1-oxyl (TEMPOL), sodium sulfite, 1,4-diazabicyclo [2.2.2]octane (DABCO), sodium pyruvate, N,N′-dimethylthiourea (DMTU), mannitol, dimethyl sulfoxide (DMSO), 6-hydroxy-2,5,7,8-tetramethylchroman-2-carboxylic acid (Trolox), 2-phenyl-1,2-benzisoselenazol-3(2H)-one (Ebselen), a-tocopherol, uric acid, sodium azide, manganese (III)-tetrakis (4-benzoic acid) porphyrin, 4,5-dihydroxybenzene-1,3-disulfonate, or a combination thereof. Other useful reactive scavengers and methods for their use in reducing photodamage or other deleterious photon-related processes are set forth in U.S. Pat. No. 10,106,851, which is incorporated herein by reference.

A fluidic medium may comprise a blocking agent. A blocking agent may include any species that inhibits orthogonal binding phenomena between assay agents and array components, such as polyethylene glycol, dextrans, albumin, or synthetic polymers such as PF-127 or polyvinylpyrrolidone.

A method set forth herein may involve a step of delivering a fluidic medium to a vessel (e.g., a flow cell, a fluidic cartridge, a reactor or microreactor, etc.) containing an array, as set forth herein. In some cases, after delivering a fluidic medium to a vessel, the fluidic medium may be incubated with an array within the vessel. Incubation of a fluidic medium with an array may be substantially quiescent. Alternatively, incubation of a fluidic medium with an array may be non-quiescent due to mixing, agitation, or circulation of the fluidic medium within or through the vessel.

A method set forth herein may involve a step of altering a fluidic medium with respect to one or more properties of the fluidic medium. Altered properties can include temperature, pH, ionic strength, and composition of the fluidic medium. In some cases, altering a fluidic medium may comprise displacing a first fluidic medium having a first property (e.g., temperature, pH, ionic strength, composition) with a second fluidic medium having a second property, in which the first property differs from the second property. In other cases, altering a fluidic medium may comprise mixing a second fluidic medium or chemical component (e.g., a solute) into a first fluidic medium. For example, a pH of a fluidic medium may be altered by adding an acid or base species to a fluidic medium in a vessel. In another example, a fluidic medium may be diluted or condensed with respect to ionic strength or concentration of a component by addition of a second fluidic medium to the vessel.

A fluidic medium may be provided at, heated to, cooled to, or maintained at a temperature of at least about-80 degrees Celsius (° C.),−50° C., −10° C., −5° C., 0° C., 5° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 35° C., 40° C., 45° C., 50° C., 60° C., 70° C., 80° C., 90° C., 95° C., or more than 95° C. Alternatively or additionally, a fluidic medium may be provided at, heated to, cooled to, or maintained at a temperature of no more than about 95° C., 90° C., 80° C., 70° C., 60° C., 50° C., 45° C., 40° C., 35° C., 30° C., 29° C., 28° C., 27° C., 26° C., 25° C., 24° C., 23° C., 22° C., 21° C., 20° C., 19° C., 18° C., 17° C., 16° C., 15° C., 14° C., 13° C., 12° C., 11° C., 10° C., 5° C., 0° C., −5° C., −10° C., −50° C., −80° C., or less than −80° C.

A fluidic medium may be provided at or adjusted to a pH of at least about 0.0, 0.5, 1.0, 1.5, 2.0, 2.5, 3.0, 3.5, 4.0, 4.5, 5.0, 5.5, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.5, 9.0, 9.5, 10.0, 10.5, 11.0, 11.5, 12.0, 12.5, 13.0, 13.5, 14.0, or more than 14.0. Alternatively or additionally, a fluidic medium may be provided at or adjusted to a pH of no more than about 14.0, 13.5, 13.0, 12.5, 12.0, 11.5, 11.0, 10.5, 10.0, 9.5, 9.0, 8.5, 8.0, 7.9, 7.8, 7.7, 7.6, 7.5, 7.4, 7.3, 7.2, 7.1, 7.0, 6.9, 6.8, 6.7, 6.6, 6.5, 6.4, 6.3, 6.2, 6.1, 6.0, 5.5, 5.0, 4.5, 4.0, 3.5, 3.0, 2.5, 2.0, 1.5, 1.0, 0.5, or less than 0.5.

A component of a fluidic medium may be provided at or adjusted to a molar concentration of at least about 0.0001 moles per liter (M), 0.001M, 0.01M, 0.02M, 0.03M, 0.04M, 0.05M, 0.06M, 0.07M, 0.08M, 0.09M, 0.1M, 0.2M, 0.3M, 0.4M, 0.5M, 0.6M, 0.7M, 0.8M, 0.9M, 1M, 1.1M, 1.2M, 1.3M, 1.4M, 1.5M, 1.6M, 1.7M, 1.8M, 1.9M, 2M, 2.1M, 2.2M, 2.3M, 2.4M, 2.5M, 2.6M, 2.7M, 2.8M, 2.9M, 3M, 3.1M, 3.2M, 3.3M, 3.4M, 3.5M, 3.6M, 3.7M, 3.8M, 3.9M, 4M, 4.1M, 4.2M, 4.3M, 4.4M, 4.5M, 4.6M, 4.7M, 4.8M, 4.9M, 5M, 5.1M, 5.2M, 5.3M, 5.4M, 5.5M, 5.6M, 5.7M, 5.8M, 5.9M, 6M, 7M, 8M, 9M or more than 10M. Alternatively or additionally, a component of a fluidic medium may be provided at or adjusted to a molar concentration of no more than about 10 M, 9M, 8M, 7M, 6M, 5.9M, 5.8M, 5.7M, 5.6M, 5.5M, 5.4M, 5.3M, 5.2M, 5.1M, 5.0M, 4.9M, 4.8M, 4.7M, 4.6M, 4.5M, 4.4M, 4.3M, 4.2M, 4.1M, 4.0M, 3.9M, 3.8M, 3.7M, 3.6M, 3.5M, 3.4M, 3.3M, 3.2M, 3.1M, 3.0M, 2.9M, 2.8M, 2.7M, 2.6M, 2.5M, 2.4M, 2.3M, 2.2M, 2.1M, 2.0M, 1.9M, 1.8M, 1.7M, 1.6M, 1.5M, 1.4M, 1.3M, 1.2M, 1.1M, 1.0M, 0.9M, 0.8M, 0.7M, 0.6M, 0.5M, 0.4M, 0.3M, 0.2M, 0.1M, 0.09M, 0.08M, 0.07M, 0.06M, 0.05M, 0.04M, 0.03M, 0.02M, 0.01M, 0.001M, 0.001M, or less than about 0.001M.

A component of a fluidic medium may be provided at or adjusted to a weight or volumetric percentage of at least about 0.0001%, 0.001%, 0.005%, 0.01%, 0.05%, 0.1%, 0.5%, 1%, 2%, 3%, 4%, 45%, 6%, 7%, 8%, 9%, 10%, 20%, 30%, 40%, 50%, or more than 50%.

Alternatively or additionally, a component of a fluidic medium may be provided at or adjusted to a weight or volumetric percentage of no more than about 50%, 40%, 30%, 20%, 10%, 9%, 8%, 7%, 6%, 5%, 4%, 3%, 2%, 1%, 0.5%, 0.1%, 0.05%, 0.01%, 0.005%, 0.001%, 0.0001%, or less than 0.0001%.

The methods, compositions and apparatus of the present disclosure are particularly well suited for use with proteins. Although proteins are exemplified throughout the present disclosure, it will be understood that other analytes can be similarly used. Exemplary analytes include, but are not limited to, biomolecules, polysaccharides, nucleic acids, lipids, metabolites, hormones, vitamins, enzyme cofactors, therapeutic agents, candidate therapeutic agents or combinations thereof. An analyte can be a non-biological atom or molecule, such as a synthetic polymer, metal, metal oxide, ceramic, semiconductor, mineral, or a combination thereof.

One or more proteins that are used in a method, composition or apparatus herein, can be derived from a natural or synthetic source. Exemplary sources include, but are not limited to biological tissues, fluids, cells or subcellular compartments (e.g., organelles). For example, a sample can be derived from a tissue biopsy, biological fluid (e.g., blood, sweat, tears, plasma, extracellular fluid, urine, mucus, saliva, semen, vaginal fluid, synovial fluid, lymph, cerebrospinal fluid, peritoneal fluid, pleural fluid, amniotic fluid, intracellular fluid, extracellular fluid, etc.), fecal sample, hair sample, cultured cell, culture media, fixed tissue sample (e.g., fresh frozen or formalin-fixed paraffin-embedded) or product of a protein synthesis reaction. A protein source may include any sample where a protein is a native or expected constituent. For example, a primary source for a cancer biomarker protein may be a tumor biopsy sample or bodily fluid. Other sources include environmental samples or forensic samples.

Exemplary organisms from which proteins or other analytes can be derived include, for example, a mammal such as a rodent, mouse, rat, rabbit, guinea pig, ungulate, horse, sheep, pig, goat, cow, cat, dog, primate, non-human primate or human; a plant such as Arabidopsis thaliana, tobacco, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a dictyostelium discoideum; a fungi such as Pneumocystis carinii, Takifugu rubripes, yeast, Saccharamoyces cerevisiae or Schizosaccharomyces pombe; or a Plasmodium falciparum. Proteins can also be derived from a prokaryote such as a bacterium, Escherichia coli, staphylococci or Mycoplasma pneumoniae; an archae; a virus such as Hepatitis C virus, influenza virus, coronavirus, or human immunodeficiency virus; or a viroid. Proteins can be derived from a homogeneous culture or population of the above organisms or alternatively from a collection of several different organisms, for example, in a community or ecosystem.

In some cases, a protein or other biomolecule can be derived from an organism that is collected from a host organism. For example, a protein may be derived from a parasitic, pathogenic, symbiotic, or latent organism collected from a host organism. A protein can be derived from an organism, tissue, cell or biological fluid that is known or suspected of being linked with a disease state or disorder (e.g., cancer). Alternatively, a protein can be derived from an organism, tissue, cell or biological fluid that is known or suspected of not being linked to a particular disease state or disorder. For example, the proteins isolated from such a source can be used as a control for comparison to results acquired from a source that is known or suspected of being linked to the particular disease state or disorder. A sample may include a microbiome or substantial portion of a microbiome. In some cases, one or more proteins used in a method, composition or apparatus set forth herein may be obtained from a single source and no more than the single source. The single source can be, for example, a single organism (e.g., an individual human), single tissue, single cell, single organelle (e.g., endoplasmic reticulum, Golgi apparatus or nucleus), or single protein-containing particle (e.g., a viral particle or vesicle).

A method, composition or apparatus of the present disclosure can use or include a plurality of proteins having any of a variety of compositions such as a plurality of proteins composed of a proteome or fraction thereof. For example, a plurality of proteins can include solution-phase proteins, such as proteins in a biological sample or fraction thereof, or a plurality of proteins can include proteins that are immobilized, such as proteins attached to a particle or solid support. By way of further example, a plurality of proteins can include proteins that are detected, analyzed or identified in connection with a method, composition or apparatus of the present disclosure. The content of a plurality of proteins can be understood according to any of a variety of characteristics such as those set forth below or elsewhere herein.

A plurality of proteins can be characterized in terms of total protein mass. The total mass of protein in a liter of plasma has been estimated to be 70 g and the total mass of protein in a human cell has been estimated to be between 100 pg and 500 pg depending upon cells type. See Wisniewski et al. Molecular & Cellular Proteomics 13:10.1074/mcp.M113.037309, 3497-3506 (2014), which is incorporated herein by reference. A plurality of proteins used or included in a method, composition or apparatus set forth herein can include at least 1 pg, 10 pg, 100 pg, 1 ng, 10 ng, 100 ng, 1 mg, 10 mg, 100 mg, 1 mg, 10 mg, 100 mg or more protein by mass.

Alternatively or additionally, a plurality of proteins may contain at most 100 mg, 10 mg, 1 mg, 100 mg, 10 mg, 1 mg, 100 ng, 10 ng, 1 ng, 100 pg, 10 pg, 1 pg or less protein by mass.

A plurality of proteins can be characterized in terms of percent mass relative to a given source such as a biological source (e.g., cell, tissue, or biological fluid such as blood). For example, a plurality of proteins may contain at least 60%, 75%, 90%, 95%, 99%, 99.9% or more of the total protein mass present in the source from which the plurality of proteins was derived. Alternatively or additionally, a plurality of proteins may contain at most 99.9%, 99%, 95%, 90%, 75%, 60% or less of the total protein mass present in the source from which the plurality of proteins was derived.

A plurality of proteins can be characterized in terms of total number of protein molecules. The total number of protein molecules in a Saccharomyces cerevisiae cell has been estimated to be about 42 million protein molecules. See Ho et al., Cell Systems (2018), DOI: 10.1016/j.cels.2017.12.004, which is incorporated herein by reference. A plurality of proteins used or included in a method, composition or apparatus set forth herein can include at least 1 protein molecule, 10 protein molecules, 100 protein molecules, 1×10⁴ protein molecules, 1×10⁶ protein molecules, 1×10⁸ protein molecules, 1×10¹⁰ protein molecules, 1 mole (6.02214076× 10²³ molecules) of protein, 10 moles of protein molecules, 100 moles of protein molecules or more. Alternatively or additionally, a plurality of proteins may contain at most 100 moles of protein molecules, 10 moles of protein molecules, 1 mole of protein molecules, 1×10¹⁰ protein molecules, 1×10⁸ protein molecules, 1×10⁶ protein molecules, 1×10⁴ protein molecules, 100 protein molecules, 10 protein molecules, 1 protein molecule or less.

A plurality of proteins can be characterized in terms of the variety of full-length primary protein structures in the plurality. For example, the variety of full-length primary protein structures in a plurality of proteins can be equated with the number of different protein-encoding genes in the source for the plurality of proteins. Whether or not the proteins are derived from a known genome or from any genome at all, the variety of full-length primary protein structures can be counted independent of presence or absence of post translational modifications in the proteins. A human proteome is estimated to have about 20,000 different protein-encoding genes such that a plurality of proteins derived from a human can include up to about 20,000 different primary protein structures. See Aebersold et al., Nat. Chem. Biol. 14:206-214 (2018), which is incorporated herein by reference. Other genomes and proteomes in nature are known to be larger or smaller. A plurality of proteins used or included in a method, composition or apparatus set forth herein can have a complexity of at least 2, 5, 10, 100, 1×10³, 1×10⁴, 2×10⁴, 3×10⁴ or more different full-length primary protein structures. Alternatively or additionally, a plurality of proteins can have a complexity that is at most 3×10⁴, 2×10⁴, 1×10⁴, 1×10³, 100, 10, 5, 2 or fewer different full-length primary protein structures.

In relative terms, a plurality of proteins used or included in a method, composition or apparatus set forth herein may contain at least one representative for at least 60%, 75%, 90%, 95%, 99%, 99.9% or more of the proteins encoded by the genome of a source from which the sample was derived. Alternatively or additionally, a plurality of proteins may contain a representative for at most 99.9%, 99%, 95%, 90%, 75%, 60% or less of the proteins encoded by the genome of a source from which the sample was derived.

A plurality of proteins can be characterized in terms of the variety of primary protein structures in the plurality including transcribed splice variants. The human proteome has been estimated to include about 70,000 different primary protein structures when splice variants ae included. See Aebersold et al., Nat. Chem. Biol. 14:206-214 (2018), which is incorporated herein by reference. Moreover, the number of the partial-length primary protein structures can increase due to fragmentation that occurs in a sample. A plurality of proteins used or included in a method, composition or apparatus set forth herein can have a complexity of at least 2, 5, 10, 100, 1×10³, 1×10⁴, 7×10⁴, 1×10⁵, 1×10⁶ or more different primary protein structures. Alternatively or additionally, a plurality of proteins can have a complexity that is at most 1×10⁶, 1×10⁵, 7×10⁴, 1×10⁴, 1×10³, 100, 10, 5, 2 or fewer different primary protein structures.

A plurality of proteins can be characterized in terms of the variety of protein structures in the plurality including different primary structures and different proteoforms among the primary structures. Different molecular forms of proteins expressed from a given gene are considered to be different proteoforms. Proteoforms can differ, for example, due to differences in primary structure (e.g., shorter or longer amino acid sequences), different arrangement of domains (e.g., transcriptional splice variants), or different post translational modifications (e.g., presence or absence of phosphoryl, glycosyl, acetyl, or ubiquitin moieties). The human proteome is estimated to include hundreds of thousands of proteins when counting the different primary structures and proteoforms. See Aebersold et al., Nat. Chem. Biol. 14:206-214 (2018), which is incorporated herein by reference. A plurality of proteins used or included in a method, composition or apparatus set forth herein can have a complexity of at least 2, 5, 10, 100, 1×10³, 1×10⁴, 1×10⁵, 1×10⁶, 5×10⁶, 1×10⁷ or more different protein structures. Alternatively or additionally, a plurality of proteins can have a complexity that is at most 1×10⁷, 5×10⁶, 1×10⁶, 1×10⁵, 1×10⁴, 1×10³, 100, 10, 5, 2 or fewer different protein structures.

A plurality of proteins can be characterized in terms of the dynamic range for the different protein structures in the sample. The dynamic range can be a measure of the range of abundance for all different protein structures in a plurality of proteins, the range of abundance for all different primary protein structures in a plurality of proteins, the range of abundance for all different full-length primary protein structures in a plurality of proteins, the range of abundance for all different full-length gene products in a plurality of proteins, the range of abundance for all different proteoforms expressed from a given gene, or the range of abundance for any other set of different proteins set forth herein. The dynamic range for all proteins in human plasma is estimated to span more than 10 orders of magnitude from albumin, the most abundant protein, to the rarest proteins that have been measured clinically. See Anderson and Anderson Mol Cell Proteomics 1:845-67 (2002), which is incorporated herein by reference. The dynamic range for plurality of proteins set forth herein can be a factor of at least 10, 100, 1×10³, 1×10⁴, 1×10⁶, 1×10⁸, 1×10¹⁰, or more. Alternatively or additionally, the dynamic range for plurality of proteins set forth herein can be a factor of at most 1×10¹⁰, 1×10⁸, 1×10⁶, 1×10⁴, 1×10³, 100, 10 or less.

The present disclosure provides compositions, apparatus and methods that are useful for detecting, characterizing and identifying proteoforms. For example, the presence or absence of a particular post-translational modification or a particular post-translationally modified amino acid can be determined. In some embodiments, a proteoform can be characterized with respect to the location(s) of one or more post-translational modifications in the amino acid sequence of the proteoform. Locations can be identified, for example, at a specific position of the amino acid sequence for the proteoform. However, in some cases, the location of a post-translational modification in a proteoform can be determined relative to a particular structural motif of the proteoform. For example, a post-translational moiety of a proteoform can be located relative to a short sequence of amino acids in the proteoform or relative to another post-translational moiety in the proteoform.

Methods of the present disclosure are particularly well suited for manipulating and detecting proteoforms. The presence or absence of post-translational modifications (PTM) can be detected using a composition, apparatus or method set forth herein. A PTM can be detected using an affinity agent that recognizes the PTM or based on a chemical property of the PTM. In some configurations, methods set forth herein can be used to differentially manipulate proteoforms based on unique molecular properties or to distinguish one proteoform from another.

A post-translational modification may be one or more of myristoylation, palmitoylation, isoprenylation, prenylation, farnesylation, geranylgeranylation, lipoylation, flavin moiety attachment, Heme C attachment, phosphopantetheinylation, retinylidene Schiff base formation, dipthamide formation, ethanolamine phosphoglycerol attachment, hypusine, beta-Lysine addition, acylation, acetylation, deacetylation, formylation, alkylation, methylation, C-terminal amidation, arginylation, polyglutamylation, polyglycylation, butyrylation, gamma-carboxylation, glycosylation, glycation, polysialylation, malonylation, hydroxylation, iodination, nucleotide addition, phosphoate ester formation, phosphoramidate formation, phosphorylation, adenylylation, uridylylation, propionylation, pyrolglutamate formation, S-glutathionylation, S-nitrosylation, S-sulfenylation, S-sulfinylation, S-sulfonylation, succinylation, sulfation, glycation, carbamylation, carbonylation, isopeptide bond formation, biotinylation, carbamylation, oxidation, reduction, pegylation, ISGylation, SUMOylation, ubiquitination, neddylation, pupylation, citrullination, deamidation, elminylation, disulfide bridge formation, isoaspartate formation, and racemization. Proteoforms can differ with regard to presence or absence of a post-translational modification, type of post-translational modification present, location of a post-translational modification, number of post-translational modifications present or combination thereof.

A post-translational modification may occur at a particular type of amino acid residue in a protein. For example, the phosphate moiety of a particular proteoform can be present on a serine, threonine, tyrosine, histidine, cysteine, lysine, aspartate or glutamate residue. In another example, an acetyl moiety of a particular proteoform can be present on the N-terminus or on a lysine of a protein. In another example, a serine or threonine residue of a proteoform can have an

O-linked glycosyl moiety, or an asparagine residue of a proteoform can have an N-linked glycosyl moiety. In another example, a proline, lysine, asparagine, aspartate or histidine amino acid of a proteoform can be hydroxylated. In another example, a proteoform can be methylated at an arginine or lysine amino acid. In another example, a proteoform can be ubiquitinated at the N-terminal methionine or at a lysine amino acid.

A post-translationally modified version of a given amino acid can include a post-translational moiety at a side chain position that is unmodified in a standard version of the amino acid. Post-translationally modified lysines can include epsilon amines attached to post-translational moieties, whereas standard lysines have epsilon amines lacking the post-translational moieties. Post-translationally modified histidines can include side-chain tertiary amines attached to post-translational moieties, whereas in standard histidines the side-chain amines are secondary amines lacking the post-translational moieties. Post-translationally modified versions of aspartates or glutamates can include side-chain carbonyls, esters or amides attached to post-translational moieties, whereas in standard versions of aspartates or glutamates the side-chains have carboxyls lacking the post-translational moieties. Post-translationally modified versions of arginines can include side-chain amines attached to post-translational moieties, whereas in standard versions of arginines the side-chain amines lack the post-translational moieties. Post-translationally modified versions of cysteines can include thioethers attached to post-translational moieties, whereas standard versions of cysteines have sulfurs lacking the post-translational moieties. Post-translationally modified versions of serines, threonines or tyrosines can include ethers or esters attached to post-translational moieties, whereas standard versions of serines, threonines or tyrosines have hydroxyls lacking the post-translational moieties.

A method of the present disclosure can include a step of removing post-translational moieties from post-translationally modified amino acids, thereby forming standard amino acids. In some cases, an enzyme can be used to remove a post-translational moiety from an amino acid. An enzyme that removes a post-translational moiety independently of amino acid sequence context surrounding the post-translationally modified amino acid can be used. In other cases, a sequence-specific enzyme can be used to remove a post-translational moiety.

A phosphatase enzyme can be used to remove a phosphate moiety from an amino acid. A broadscale (e.g., sequence agnostic) phosphatase such as alkaline phosphatase can be useful.

Protein phosphatases are available for removing phosphate moieties from various types of amino acids. Exemplary protein phosphatases include, but are not limited to, tyrosine-specific kinases such as PTP1B; serine/threonine-specific phosphatases such as PP2C and PPP2CA; dual specificity phosphatases such as lambda protein phosphatase or VHR, both of which can remove phosphate moieties from serine, threonine or tyrosine residues; or histidine phosphatase such as PHP. Phosphatases or kinases that are specific to particular signal transduction pathways can be used to remove phosphates in a sequence specific manner if desired.

Several enzymes are available for removing post-translational moieties from lysines. Examples are set forth in Wang and Cole, Cell Chemical Biology 27:953-969 (2020) (which is incorporated herein by reference) and below. Lysine deacetylases can be used to remove acetyl moieties from lysines. For example, at least eighteen different protein lysine deacetylases (e.g., histone deacetylases) are known to remove acetyl moieties from lysines in human proteins. Lysine demethylases can be used to remove methyl moieties from lysines. Deubiquitinases (DUBs) are isopeptidases that sever the amide bond between a lysine side chain of a protein and the ubiquitin (Ub) C terminus. Many DUBs can cleave Ub-Ub amide linkages whereas others show selectivity for particular ubiquitinated proteins.

Optionally, glycan moieties can be released from proteins in a method of the present disclosure. For example, N-glycans or O-glycans can be released from glycoproteins using glycosidases. Any of a variety of enzymes can be used to remove glycans from proteins. For example, a-2-3,6,8,9-Neuraminidase can be used to cleave non-reducing terminal branched and unbranched sialic acids; β-1,4-galactosidase can be used to remove β-1,4-linked nonreducing terminal galactose from proteins; β-N-acetylgucosaminidase can be used to cleave non-reducing terminal β-linked N-acetylgucosamine from proteins; endo-a-N-acetylgalactosaminidase can be used to remove O-glycosylation, for example, removing serine- or threonine-linked unsubstituted Galb 1,3GalNac; and PNGase F can be used to cleave oligosaccharides from asparagines.

Exemplary reagents and methods for releasing glycans from proteins are set forth in Zhang et al. Frontiers in Chemistry, vol 8, Article 508 (2020) doi: 10.3389/fchem.2020.00508, which is incorporated herein by reference.

A plurality of extant proteins may contain two or more proteoforms of a single species of protein (e.g., at least 2, 3, 4, 5, 10, 20, 50, 100, or more than 100 proteoforms). Alternatively, a plurality of extant proteins may contain only a single proteoform of a single species. A plurality of extant proteins may contain at least one species of protein having two or more proteoforms (e.g., at least 2, 10, 50, 100, 500, 1000, 5000, 10000, or more than 10000 species of protein having two or more proteoforms). Alternatively, a plurality of extant proteins may contain at least one species of protein having only one proteoform (e.g., at least 2, 10, 50, 100, 500, 1000, 5000, 10000, or more than 10000 species of protein having only one proteoform).

A method of identifying extant proteins may further include identifying proteoforms of extant proteins. Accordingly, a method of identifying a proteoform of an individual protein can include the steps of: i) identifying a primary amino acid sequence of the protein based upon a binding profile of the protein, thereby identifying the protein, and ii) identifying a proteoform of the protein. Proteoform-specific affinity agents may be useful for identifying the proteoform of an extant protein. A proteoform-specific affinity agent can be a promiscuous affinity agent, for example binding to post-translational modifications (e.g., methylations, phosphorylations, glycosylations, etc.) of a plurality of protein species and/or proteoforms. A proteoform-specific affinity agent can be highly specific to a single proteoform of one or more protein species (e.g., only binding to a single post-translationally modified amino acid of a single protein species). A proteoform may be identified in part by detecting presence of binding of one or more affinity agents to an extant protein. Alternatively, a proteoform may be identified in part by an absence of detectable binding of one or more affinity agents to an extant protein (e.g., due to absence of a post-translational modification at an amino acid residue of the extant protein, due to absence of a bindable epitope due to splice variation of the extant protein, etc.).

In some cases, it may be preferable to contact extant proteins with a proteoform-specific affinity agent before contacting the extant proteins with other promiscuous or non-proteoform affinity agents. Presence of certain post-translational modification may inhibit binding of affinity agents to epitopes where said post-translational modification are present. Accordingly, a method may further comprise a step of removing post-translation modification (e.g., chemically or enzymatically) from extant proteins. After detecting binding of proteoform-specific affinity agents to extant proteins, and optionally removing one or more post-translational modification from the extant proteins, the extant proteins may be subsequently contacted with a series of promiscuous affinity agents, thereby providing binding profiles for each individual extant protein.

### EXAMPLES

### Example 1. Probe Library for Protein Characterization

A probe library is provided to an analyte characterization system. The probe library is provided in a 384-well plate having a 16 row by 24 column arrangement. Each well of the well plate contains a probe composition comprising a plurality of detectable probes in a buffered solution. The probe library contains 300 unique multi-affinity probe compositions for analyte characterization, plus duplicates of 60 of the 300 unique multi-affinity probe compositions. The remaining 24 wells of the probe library contain various standard and control reagents, including positive control single-affinity probes, negative control probes, and fluorometric standards. The wells of the first row of the probe library contain the standard and control reagents. The wells of the second through sixteenth rows contain the multi-affinity probe compositions. Each row contains four sets of probe compositions, each set containing five unique probe compositions followed by a duplicate of the first probe composition (i.e., Set 1: composition 1, composition 2, composition 3, composition 4, composition 5, composition 1; Set 2: composition 6, composition 7, composition 8, composition 9, composition 10, composition 6; etc.).

The probe compositions of row 2 of the probe library all utilize nucleic acid particles as retaining components for joining affinity reagents to fluorescent labels. Each probe comprises an antibody joined to 8 fluorescent dye molecules by a nucleic acid origami particle. The nucleic acid particle has a first face to which the antibody is attached and a second face to which the fluorescent dye molecules are attached. The probe compositions of row 3 of the probe library all utilize dendrimeric polymers as retaining components for joining affinity reagents to fluorescent labels. Each probe comprises an antibody joined to 8 fluorescent dye molecules by a PAMAM dendrimer particle.

The probe kit is utilized by the system for a method of analyte characterization. The system contains an automated pipetting system that individually transfers the probe compositions from individual wells of the probe library to a fluidic cartridge containing a single-analyte array of full-length protein molecules. The ordering of fluid transfer goes sequentially according to the columns (i.e., column 1, column 2, etc.), beginning with the standard or control probe composition, followed by the remaining fifteen multi-affinity probe compositions of the column. After incubation with the single-analyte array, binding of probes of a given probe composition to individual analytes is detected at single-analyte resolution. After detection, probes are removed from the fluidic cartridge, and a new probe composition is delivered to the fluidic cartridge. After all 384 probe compositions are detected, binding data is utilized to infer identities of different full-length proteins of the single-analyte array.

### Example 2. Probe Library for Protein Characterization

The probe library of Example 1 is provided to the analyte characterization system. The probe compositions of row 4 of the probe library all utilize secondary antibodies, each secondary antibody labeled with at least 6 fluorescent dye molecules. Each probe composition contains a mixture of primary multi-affinity antibodies and single-affinity secondary antibodies. The probe compositions of row 5 of the probe library all utilize secondary antibody fragments, each secondary antibody fragment labeled with at least 6 fluorescent dye molecules. Each probe composition contains a mixture of primary multi-affinity antibodies and single-affinity secondary antibody fragments. The secondary-labeled multi-affinity probe compositions of rows 4 and 5 are utilized in the method described in Example 1.

### Example 3. Probe Library for Protein Characterization

The probe library of Example 1 is provided to the analyte characterization system. The probes of row 4 are grouped into four sets according to optimal probe concentration for the analyte characterization assay. The concentration is chosen to be about double the empirically measured dissociation constant (K_{D}) of each probe. The probe compositions of the first set of probes of row 4 (columns 1 through 6) each have a probe concentration of about 200 nanomolar (nM). The probe compositions of the second set of probes of row 4 (columns 7 through 12) each have a probe concentration of about 250 nanomolar (nM). The probe compositions of the third set of probes of row 4 (columns 13 through 18) each have a probe concentration of about 300 nanomolar (nM). The probe compositions of the fourth set of probes of row 4 (columns 19 through 24) each have a probe concentration of about 400 nanomolar (nM). The four sets of probe compositions of differing concentrations are utilized in the method described in Example 1.

### Example 4. Probe Library for Protein Characterization

The probe library of Example 1 is provided to the analyte characterization system. The probes of row 2 are provided in a buffer composition that differs from the buffer composition of the probes of row 3. Due to the presence of nucleic acid particles in the probe compositions of row 2, the probes are provided in a buffer containing a higher concentration of magnesium chloride than the buffers of row 3 that are substantially devoid of nucleic acid particles. The probe compositions of differing buffer formulations are utilized in the method described in Example 1.

### Example 5. Probe Library for Protein Characterization

A probe library is provided to an analyte characterization system. The probe library contains the same 300 unique multi-affinity probes as described in Examples 1 through 4. Some wells contain multiplexed probe compositions and other wells contain single-plex probe compositions. The multiplexed well compositions contain two differing multi-affinity probes, the probes differing with respect to binding affinity for an epitope target. Probes of a first type of multi-affinity probe are distinguished from probes of a second type of multi-affinity probe by differing fluorescent labels. Each row contains four sets of six probe compositions, each set of probe compositions having the pattern: {probe 1/probe 2; probe 2/probe 3; probe 3/probe 4; probe 4/probe 5; probe 1 only, probe 5 only}. Row 1 of the probe library contains the 24 control and standard probe compositions, as described in Example 1. The probe compositions of the probe library are utilized in a method similar to the method described in Example 1, with dual-color detection utilized to simultaneously identify analytes bound by either the first type of probe or second type of probe of the multiplexed probe compositions.

### Example 6. Probe Library for Protein Isoform Characterization

A probe library is provided to an analyte characterization system. The probe library contains a series of probe compositions that are utilized to detect 6 differing isoforms of the human MAPT protein. The probe library contains a first multi-affinity probe that binds to any isoform of the MAPT protein. The probe library further contains five different single-affinity probes, each single-affinity probe binding a different isoform of MAPT (the sixth isoform can be identified via binding of the multi-affinity probe but no binding of any of the single-affinity probes). The probe library further comprises 2 multi-affinity probes that recognize a short (2 to 4 amino acid) epitope that would be present only if the 1N or 2N inserts are present. Each of the 8 different probes are provided to the library in triplicate.

The probe kit is utilized by the system for a method of analyte characterization. The system contains an automated pipetting system that individually transfers the probe compositions from individual wells of the probe library to a fluidic cartridge containing a single-analyte array containing different MAPT molecules from a brain cell lysate. After incubation with the single-analyte array, binding of probes of a given probe composition to individual analytes is detected at single-analyte resolution. After detection, probes are removed from the fluidic cartridge and a new probe composition is delivered to the fluidic cartridge. After all 24 probe compositions are detected, binding data is utilized to infer isoforms of different full-length MAPT proteins of the single-analyte array.

### Example 7. Probe Library for Protein Characterization

A probe library is provided to an analyte characterization system. The probe library is provided in a 384-well plate having a 16 row by 24 column arrangement. Each well of the well plate contains a probe composition comprising a plurality of detectable probes in a buffered solution. The probe library contains 300 unique multi-affinity probe compositions for analyte characterization, plus duplicates of 21 of the 300 unique multi-affinity probe compositions. The probe library further comprises 39 single-affinity probes (triplicate of 13 different single-affinity probes), each unique probe recognizing a different post-translationally modified amino acid (methyl-histidine, methyl-glutamate, methyl-glutamine, methyl-asparagine, phospho-serine, phospho-threonine, phospho-tyrosine, phospho-histidine, ubiquitin (ubi)-lysine, ubi-cysteine, ubi-serine, ubi-threonine, and ubi-N-terminus). The remaining 24 wells of the probe library contain various standard and control reagents, including positive control single-affinity probes, negative control probes, and fluorometric standards. The wells of the first row of the probe library contain the standard and control reagents. The wells of the second through sixteenth rows contain the multi-affinity probe compositions and single-affinity probe compositions.

The probe kit is utilized by the system for a method of analyte characterization. The system contains an automated pipetting system that individually transfers the probe compositions from individual wells of the probe library to a fluidic cartridge containing a single-analyte array of full-length protein molecules. The ordering of fluid transfer goes sequentially according to the columns (i.e., column 1, column 2, etc.), beginning with the standard or control probe composition, followed by the remaining fifteen multi-affinity probe compositions of the column. After incubation with the single-analyte array, binding of probes of a given probe composition to individual analytes is detected at single-analyte resolution. After detection, probes are removed from the fluidic cartridge, and a new probe composition is delivered to the fluidic cartridge. After all 384 probe compositions are detected, binding data is utilized to infer identities of different full-length proteins of the single-analyte array, and to further identify presence or absence of certain post-translationally modified amino acid residues for each identified protein molecule.

1. A kit, comprising: a plurality of joined vessels, wherein each vessel is fluidically isolated from each other vessel of the plurality of vessels, and wherein the plurality of joined vessels comprises a plurality of probe compositions, wherein each vessel of the plurality of joined vessels comprises a unique probe composition of the plurality of probe compositions, and wherein: (i) each vessel of a first set of vessels of the plurality of joined vessels comprises a multi-affinity probe composition; and (ii) each vessel of a second set of vessels of the plurality of joined vessels comprises a single-affinity probe composition.

2. The kit of claim 1, wherein the plurality of joined vessels comprises at least 20 vessels.

3. The kit of claim 1, wherein a probe composition of the plurality of probe compositions comprises a fluidic medium.

4. (canceled)

5. The kit of claim 1, wherein probes of the multi-affinity probe composition have a binding specificity for two or more different proteins as determined by primary amino acid sequence.

6. The kit of claim 1, wherein probes of the multi-affinity probe composition have a binding specificity for an epitope of 2 to 5 amino acids in length.

7. The kit of claim 6, wherein the epitope is common to two or more differing proteins.

8. The kit of claim 1, wherein probes of the single-affinity probe composition have a binding specificity for a single protein, as determined by primary amino acid sequence.

9. The kit of claim 1, wherein probes of the single-affinity probe composition have a binding specificity for a single proteoform or isoform of a single protein.

10. The kit of claim 1, wherein the first set of vessels comprises at least 80% of vessels of the plurality of joined vessels.

11. The kit of claim 1, wherein the first set of vessels comprises no more than 95% of vessels of the plurality of joined vessels.

12. The kit of claim 1, wherein probes of the multi-affinity probe composition have a same probe architecture as probes of the single-affinity probe composition.

13. The kit of claim 1, wherein probes of the multi-affinity probe composition have a differing probe architecture as probes of the single-affinity probe composition.

14. The kit of claim 1, wherein the multi-affinity probe composition has a same fluid composition as the single-affinity probe composition.

15. The kit of claim 1, wherein the multi-affinity probe composition has a differing fluid composition from the single-affinity probe composition.

16. The kit of claim 1, wherein probes of a first multi-affinity probe composition of the first set of vessels have a differing probe architecture from probes of a second multi-affinity probe composition of the first set of vessels.

17. The kit of claim 1, wherein a first multi-affinity probe composition of the first set of vessels has a differing fluid composition from a second multi-affinity probe composition of the first set of vessels.

18. The kit of claim 1, wherein probes of a first single-affinity probe composition of the second set of vessels have a differing probe architecture from probes of a second single-affinity probe composition of the second set of vessels.

19. The kit of claim 1, wherein a first single-affinity probe composition of the second set of vessels has a differing fluid composition from a second single-affinity probe composition of the second set of vessels.

20.-21. (canceled)

22. A method of characterizing an analyte, comprising:
(a) sequentially contacting each probe composition of a probe library, wherein the probe library comprises a plurality of joined vessels, wherein each vessel is fluidically isolated from each other vessel of the plurality of vessels, and wherein the plurality of joined vessels comprises a plurality of probe compositions, wherein each vessel of the plurality of joined vessels comprises a unique probe composition of the plurality of probe compositions, and wherein: (i) each vessel of a first set of vessels of the plurality of joined vessels comprises a multi-affinity probe composition; and (ii) each vessel of a second set of vessels of the plurality of joined vessels comprises a single-affinity probe composition;
(b) for each probe composition contacted to the array of analytes, detecting presence or absence of binding of a probe of the probe composition to each of the single analytes of the array of analytes at single-analyte resolution; and
(c) for each single analyte of the array of analytes, characterizing the single analyte based upon the presence or absence of binding of each probe composition to the single analyte.

23. A method of characterizing an analyte, comprising:
(a) an array of analytes, wherein each analyte of the array of analytes is individually addressable at single-analyte resolution;
(b) a probe library, wherein the probe library comprises a plurality of joined vessels, wherein each vessel is fluidically isolated from each other vessel of the plurality of vessels, and wherein the plurality of joined vessels comprises a plurality of probe compositions, wherein each vessel of the plurality of joined vessels comprises a unique probe composition of the plurality of probe compositions, and wherein: (i) each vessel of a first set of vessels of the plurality of joined vessels comprises a multi-affinity probe composition; and (ii) each vessel of a second set of vessels of the plurality of joined vessels comprises a single-affinity probe composition;
(c) a fluidic system that provides fluidic communication between the array of analytes and each probe composition of the probe library;
(d) a detection device that detects presence or absence of a probe of the probe library co-localized with each single analyte of the array of analytes; and
(e) a processor that receives detection data from the detection device and, based upon the detection data, characterizes each individual analyte of the array of analytes.
